SEQUENCE LISTING (1 sequence)

SEQ ID NO: 1 - protein, 163 aa, synthetic construct
  REGION 1..163: note = amino acid residues of Pin1
MADEEKLPPGWEKRMSRSSGRVYYFNHITNASQWERPSGNSSSGGKNGQGEPARVRCSHLLVKHSQSRRPSSWRQEKITRTKEEALELINGYIQKIKSGEEDFESLASQFSDCSSAKARGDLGAFSRGQMQKPFEDASFALRTGEMSGPVFTDSGIHIILRTE

(19) United States
(12) Patent Application Publication
(10) Pub. No.: US 2023/0406837 A1
(43) Pub. Date: Dec. 21, 2023

(54) SULFAMATE MODULATORS OF PIN1 ACTIVITY AND USES THEREOF

(71) Applicant: Yeda Research and Development Co., Ltd., Rehovot (IL)

(72) Inventors: Nir LONDON, Rehovot (IL); Rambabu Reddi, Rehovot (IL)

(73) Assignee: Yeda Research and Development Co., Ltd., Rehovot (IL)

(21) Appl. No.: 18/334,385

(22) Filed: Jun. 14, 2023

Related U.S. Application Data
(60) Provisional application No. 63/384,965, filed on Nov. 24, 2022.
(60) Provisional application No. 63/351,927, filed on Jun. 14, 2022.

(51) Int. Cl. C07D 333/48

(52) U.S. Cl. CPC C07D 333/48

(57) ABSTRACT

Provided herein are Sulfamate compounds for use in modulating an activity of peptidyl-prolyl isomerase NIMA-interacting-1 (Pin1).

### CROSS REFERENCE TO RELATED APPLICATIONS

This application claims the benefit United States Provisional Application Nos 63/384,965, filed on 24 Nov. 2022 and 63/351,927, filed 14 Jun. 2022 which are incorporated in their entirety herein by reference.

### SEQUENCE LISTING STATEMENT

The instant application contains a Sequence Listing which has been submitted electronically in XML file format and is hereby incorporated by reference in its entirety. Said XML copy, created on Jun. 13, 2023, is named P-618986-US_SL.xml and is 4,587 bytes in size.

### FIELD OF INTEREST

This invention is directed to sulfamate compounds for use in modulating an activity of peptidyl-prolyl isomerase NIMA-interacting-1 (Pin1).

### BACKGROUND

The present invention, in some embodiments thereof, relates to pharmacology, and more particularly, but not exclusively, to newly designed compounds that covalently bind to, and/or modulate the activity of, Pin1 and to uses thereof, for example, in treating diseases associated with Pin1 activity.

### Peptidyl-Prolyl Isomerase NIMA-Interacting-1 (Pin1)

Phosphorylation of Serine-Proline or Threonine-Proline motifs (pSer/Thr-Pro) by proline-directed kinases is a central signaling mechanism that is reported to be frequently deregulated in oncogenic pathways, driving cell transformation and downregulating apoptosis [Hanahan & Weinberg, Cell 2011, 144:646-674]. This motif can be isomerized (from cis to trans or trans to cis) by peptidyl-prolyl isomerase NIMA-interacting-1 (Pin1) [Lu and Zhou, Nat Rev Mol Cell Biol 2007, 8:904-916], which is the only phosphorylation-dependent isomerase amongst the approximately 30 peptidyl-prolyl cis-trans isomerases (PPIases) in the human proteome. This isomerization induces conformational changes that can impact substrate stability [Lam et al., Mol Cancer 2008, 7:91; Liao et al., Oncogene 2009, 28:2436-2445; Lee et al., Nat Cell Biol 2009, 11:97-105], activation [Chen et al., Cell Death Dis 2018, 9:883], subcellular localization [Ryo et al., Nat Cell Biol 2001, 3:793-801], and/or binding to interaction partners including Proline-directed kinases and phosphatases, which are mostly trans-specific [Xiang et al., Nature 2010, 467:729-733; Zhou et al., Mol Cell 2000, 6:873-883; Brown et al., Nat Cell Biol 1999, 1:438-443]. Pin1 is therefore an important mediator of proline-directed signaling networks, and frequently plays a role in cancer, of activating oncogenes and inactivating tumor suppressors [Chen et al., Cell Death Dis 2018, 9:883].

Several lines of evidence indicate that abnormal Pin1 activation is a key driver of oncogenesis.

Pin1 has been reported to be overexpressed and/or overactivated in at least 38 tumor types [Bao et al., Am J Pathol 2004, 164:1727-1737], by mechanisms which include transcriptional activation [Rustighi et al., Nat Cell Biol 2009, 11:133-142; Ryo et al., Mol Cell Biol 2002, 22:5281-5295] and post-translational modifications [Lee et al., Mol Cell 2011, 42:147-159; Rangasamy et al., Proc Natl Acad Sci 2012, 109:8149-8154; Chen et al., Cancer Res 2013, 73: 3951-3962; Eckerdt et al., J Biol Chem 2005, 280:36575-36583]. High expression is reported to correlate with poor clinical prognosis [Lu, Cancer Cell 2003, 4:175-180; Tan et al., Cancer Biol Ther 2010, 9:111-119], whereas polymorphisms that result in lower Pin1 expression is reported to reduce cancer risk [Li et al., PLoS One 2013, 8:e68148].

Pin1 has been reported to sustain proliferative signaling in cancer cells by upregulating over 50 oncogenes or growth-promoting factors [Chen et al., Cell Death Dis 2018, 9:883], including NF-κB [Ryo et al., Mol Cell 2003, 12:1413-1426], c-Myc [Farrell et al., Mol Cell Biol 2013, 33:2930-2949] and Notch1 [Rustighi et al., Nat Cell Biol 2009, 11:133-142], while suppressing over 20 tumor suppressors or growth-inhibiting factors, such as FOXOs [Brenkman et al., Cancer Res 2008, 68:7597-7605], Bcl2 [Basu et al., Neoplasia 2002, 4:218-227] and RARα [Gianni et al., Cancer Res 2009, 69:1016-1026].

Furthermore, Pin1 depletion was reported to inhibit tumorigenesis in mouse models derived by mutated p53 [Girardini et al., Cancer Cell 2011, 20:79-91], activated HER2/RAS [Wulf et al., EMBO J 2004, 23:3397-3407], or constitutively expressed c-Myc [D'Artista et al., Oncotarget 2016, 7:21786-21798].

However, Pin1's potential as drug target remains elusive because available Pin1 inhibitors lack the specificity and/or cell permeability to interrogate its pharmacological function in vivo [Lu & Hunter, Cell Res 2014, 24:1033-1049; Moore & Potter, Bioorganic Med Chem Lett 2013, 23:4283-4291; Fila et al., J Biol Chem 2008, 283:21714-21724].

### Electrophilic Organic Compounds

Electrophilic organic compounds play a pivotal role in chemical biology by reacting with nucleophilic amino acids like cysteine, lysine, tyrosine, etc [Ray, S. & Murkin, A. S. Design. Biochemistry 58, 5234-5244 (2019)]. Some of these electrophiles have successfully been used in bioconjugation for the synthesis of antibody-drug conjugates [Gehringer, M. & Laufer, S. A. J. Med Chem. 62, 5673-5724 (2019); Drago, J. Z., Modi, S. & Chandarlapaty, S. Nat. Rev. Clin. Oncol. 18, 327-344 (2021)], proteomics [Khongorzul, P., Ling, C. J., Khan, F. U., Ihsan, A. U. & Zhang, J. Review. Mol. Cancer Res. 18, 3-19 (2020)], activity based protein profiling (ABPP), and as covalent warheads in the designing of targeted covalent inhibitors (TCIs) [Backus, K. M. et al. Nature 534, 570-574 (2016)]. In spite of the therapeutic benefits of covalent inhibitors like enhanced and sustained pharmacological potency and protein isoform selectivity compared to their reversible counterparts, their high toxicity due to the off-target reactivity is a key concern [Gehringer, M. & Laufer, S. A. J. Med. Chem. 62, 5673-5724 (2019); Chem. Soc. Rev. 49].

Some of the most commonly used electrophiles in designing targeted covalent inhibitors are acrylamides and chloroacetamides which react with cysteines (FIG. 1A) [Flanagan, M. E. et al. J. Med Chem. 57, 10072-10079 (2014); Resnick, E. et al. J. Am. Chem. Soc. 141, 8951-8968 (2019); Gabizon, R., Resnick, E. & London, N. in The Discovery and Utility of Chemical Probes in Target Discovery 69-99 (2020); Henning, N. J. et al. J. Am. Chem. Soc. 144, 701-708 (2022).].

While acrylamide-based electrophiles are known to be able to achieve sufficiently low reactivity, chloroacetamides are more reactive [Flanagan, M. E. et al. J. Med Chem. 57, 10072-10079 (2014); Resnick, E. et al. J. Am. Chem. Soc. 141, 8951-8968 (2019)] as covalent ‘warheads’. This greatly limits their application in designing targeted covalent inhibitors (TCIs). Consequently, fluorochloro-acetamide, α-substituted chloroacetamides [Gehringer, M. & Laufer, S. A. J. Med Chem. 62, 5673-5724 (2019); Allimuthu, D. & Adams, D. J. ACS Chem. Biol. 12, 2124-2131 (2017)] and di- and tri halo acetamide [Ma, C.; Xia, Z.; Sacco, M. D.; Hu, Y.; et al. J. Am. Chem. Soc. 2021, 143 (49), 20697-20709.] warheads have been reported as less reactive alternatives (FIG. 1B). Although these warheads showed improved selectivity, it was typically at the cost of reduced potency. Tunability of the electrophile reactivity can help to find the optimal balance between selectivity and potency. However, there are very few degrees of freedom with chloroacetamides. All these chemistries are covalently labeled to POI but do not release ‘payloads’ in the cells.

### Sulfamate Substitution Functionality

Sulfamate (—O—SO₂—NR—) functionality has a pivotal role in medicinal chemistry and many bioactive and drug molecules contain this functionality [Winum, J. Y., Scozzafava, A., Montero, J. L. & Supuran, C. T. Med Res. Rev. 25, 186-228 (2005).].

Sulfopin was developed as a selective covalent inhibitor of Pin1 which blocks Myc-driven tumors in vivo [Dubiella, C. et al. Nat. Chem. Biol. 17, 954-963 (2021)].

Herein, provided are Sulfamate compounds as highly stable warheads with tunable reactivity and similar geometry to chloroacetamides. (FIG. 1).

### SUMMARY

In some embodiments, provided herein is a Sulfamate compound represented by the structure of Formula I:

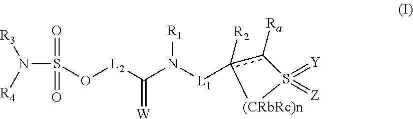

or a pharmaceutically acceptable salt thereof,
wherein:
the [symbol] represents a saturated or non-saturated bond;
Y and Z are each independently selected from the group consisting of O, S and NH;
   each of R₂, Ra, Rb and Rc are independently selected from the group consisting of hydrogen, alkyl, alkenyl, alkynyl, cycloalkyl, aryl, heteroaryl, heterocyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, or alternatively, R₂ is absent when the dashed line represents an unsaturated bond;
   each of R₁, R₃ and R₄ are independently selected from the group consisting of hydrogen, OH, any group that forms a urea, an amide, a thiourea, hydrazide or hydroxamic acid (via the nitrogen), substituted or unsubstituted linear or branched alkyl, alkenyl, alkynyl, substituted or unsubstituted cycloalkyl, substituted or unsubstituted heterocyclyl, substituted or unsubstituted aryl, and substituted or unsubstituted heteroaryl, or R₃ and R₄ forms together a five or six membered ring with the nitrogen;
   n is 1, 2, 3 or 4;
   L₁ is a bond, substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group;
   L₂ is substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group;
   W is O, S or NRs; and
   R₅ is selected from the group consisting of hydrogen, alkyl, alkenyl, alkynyl, cycloalkyl, heterocyclic, aryl and heteroaryl.

In some embodiments, provided herein is a Sulfamate compound represented by the structure of Formula II:

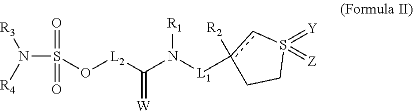

or a pharmaceutically acceptable salt thereof,
wherein,
R₁, R₂, R₃, R₄, L₁, L₂, W, Y and Z are defined in Formula I.

In some embodiments, this invention is directed to Sulfamate compound represented by the structure of Formula III:

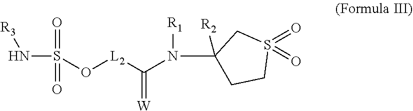

or a pharmaceutically acceptable salt thereof,
wherein,
R₁, R₂, R₃, L₂, and W are as defined in Formula I.

In some embodiments, provided herein is a Sulfamate compound represented by the structure of Formula IV:

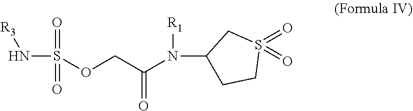

or a pharmaceutically acceptable salt thereof,
wherein,
R₁ and R₃ are defined in Formula I.

In some embodiments, provided herein is a pharmaceutical composition comprising the compound of Formula I, II, III, IV or a pharmaceutically acceptable salt thereof.

In some embodiments, provided herein is a method of treating a disease or condition associated with Pin1 activity, comprising administering a compound of Formula I, II, III, or IV or a pharmaceutically acceptable salt thereof, or a pharmaceutical composition comprising a compound of Formula I, II, III, or IV or a pharmaceutically acceptable salt thereof.

### BRIEF DESCRIPTION OF THE DRAWINGS

The subject matter directed to the sulfonate and sulfamate compounds and uses thereof is particularly pointed out and distinctly claimed in the concluding portion of the specification. The synthetic compounds and uses thereof, however, both as to organization and method of operation, together with objects, features, and advantages thereof, may best be understood by reference to the following detailed description when read with the accompanying drawings in which:

FIG. 1A: Structures of acrylamide and chloroacetamide-based covalent inhibitors. Ibrutinib (BTK inhibitor), Afatinib (EGFR inhibitor), Sotorasib (KRAS^{G12C} inhibitor), RSL3 (GPX4 inhibitor), KT53 (Glutathione S-Transferase Omega 1 inhibitor), and Sulfopin (Pin1 inhibitor).

FIG. 1B: Reactivity pattern of alpha-substituted acetamide.

FIG. 1C: schematic presentation of the reaction of cysteine 113 of PIN, and the sulfamate compound of this invention.

FIG. 1D: schematic presentation of recognition, of sulfamate compound of this invention and Pin1.

FIG. 2: Synthetic schemes of model compounds 1a-1j.

FIG. 3: Synthetic schemes of model compounds 2a-2c.

FIGS. 4A-4E: α-Sulfamate acetamides show varied reactivity profiles. FIG. 4A: LC chromatogram shows monitoring of the reaction of 1d (200 μM) with GSH (5 mM) at 0 h and 5 h. GSH adduct: Retention time (RT)=4.7 min, m/z=465; 1d: RT=5.45 min, m/z=253; reference: RT=5.8 min. UV absorption was measured between 220-400 nm. FIG. 4B: Rates of depletion of model compounds (1a-1g) in a reaction between 200 μM compound and 5 mM GSH in PBS buffer at pH 8, 14° C. (n=2) for 6 h. FIG. 4C: Estimation of intrinsic thiol reactivity as determined by a DTNB assay (see methods). FIG. 4D: The second-order reaction rate constants for reaction with DTNB. FIG. 4E: Possible resonance structures for the sulfamate compounds.

FIGS. 5A-5C: α-sulfamate acetamide 1a-aj, and 2a-2c can show up to two orders of magnitude less reactivity towards GSH than chloroacetamide. FIG. 5A: Chemical structures of model α-sulfamate/sulfonate/sulfone acetamides. FIG. 5B: Half-life (t_{1/2}) of the model compounds (1a-1j) assessed by GSH consumption assay via LC/MS (FIG. 4B). FIG. 5C: In situ proteomic labeling with alkyne probes (2a-2c). Mino cells were treated for 2 h with either DMSO or 2a-c, then lysed, reacted with TAMRA-azide using CuAAC, and imaged via in-gel fluorescence (532 nm). A band that is selectively detected only by compound 2a is indicated by *.

FIG. 6: GSH chromatograms for all the model compounds: UV chromatogram (220-400 nm) of the LC/MS traces of model compounds (200 μM; BnA, 1a-1h) incubated with reduced glutathione (5 mM) and 4-nitrocyano benzene (100 μM) in PBS buffer at pH 8, 14° C. at the last measured point of the GSH t_{1/2} experiment (FIG. 4B).* Impurity obtained from the column during the assay with 1d, 1e and 1g.

FIG. 7: lysine reactivity of model compounds: UV chromatogram (220-400 nm) of the LC/MS traces of model compounds (200 μM; BnA, 1a, 1b, 1d and 1e) incubated with N-acyl lysine methyl ester (5 mM) and 4-nitrocyano benzene (100 μM) in PBS buffer at pH 8, 37° C., 4 days. No significant reaction has been observed with model compounds. “*” refers to hydrolysis product. “#” refers to 1a product formation.

FIG. 8: Buffer stability of model compounds. UV chromatogram (220-400 nm) of the LC/MS traces of model compounds (200 μM; 1a-1h and 1j) incubated with 4-nitrocyano benzene (100 μM) in PBS buffer at pH 8, 37° C. The percentage of hydrolysis was quantified using LC/MS at a wavelength of 220 nm. 1a, 1b, and 1c underwent 12, 26, and 51% hydrolysis. A peak at 3.5 min is a contamination of the column. ** hydrolysis product. # For compounds 1b and 1c, the sulfonates were substituted with chloride ions.

FIG. 9: Synthetic schemes of Sulfamate compounds of this invention (4a-4g).

FIG. 10: Pin1 labeling with sulfonate (4a) and sulfamate (4b-4g) compounds. Deconvoluted MS spectra (intact protein LC/MS) of 2 μM Pin1 incubated with 2 μM sulfonate and sulfamate compounds (4a-4g) at pH 7.5, 25° C. after 1 h.

FIGS. 11A-11E: Sulfamate acetamides as potent and selective Pin1 inhibitors. FIG. 11A. Chemical structures of the sulfonate and sulfamate compounds (4a-4g). FIG. 11B. Deconvoluted LC/MS spectrum for Pin1 (2 μM) incubated with 4 g at pH 7.5, 25° C., 1 h. The adduct mass corresponds to a labeling event in which the sulfamate group was released. FIG. 11C. Percent of Pin1 labeling (2 μM) with the probes (4a-4g; 2 μM; y-axis) compared to their intrinsic thiol reactivity as assessed by their rate of reaction in a DTNB assay (x-axis). FIG. 11D. Cellular engagement of the Sulfamates. OCI-AML2 cells were treated with DMSO, Sulfopin or sulfamate compounds (4c, 4d, 4g) at 0.5 and 2.5 μM concentration for 4 hours. Lysates were then incubated with a Sulfopin desthiobiotin (DTB) probe (1 μM), pulled down using streptavidin beads before running a western blot against Pin1. FIG. 11E. IsoDTB ABPP experiment with sulfopin and sulfamate compound 4d. PATU-8988T cells were treated with 2.5 μM compound for 4 h followed by incubation of Iodo-acetamide alkyne and CuAAC click reaction with heavy/light azides containing DTB tags. The labeled peptides were pull-down with streptavidin beads and analysed by LC/MS/MS (similar protocol to BTK, see FIG. 521; n=4). Proteins in the box have a heavy to light (H/L) ratio ≥2. Only peptides detected in three out of four repetitions are presented.

FIGS. 12A-12B: Thiol reactivity of sulfonate and sulfamate compounds. FIG. 12A. Loss of DTNB absorbance over time as a function of intrinsic thiol reactivity of sulfonate and sulfamate compounds. FIG. 12B. The second-order rate constants for the reaction as extracted from the DTNB thiol reactivity assay (see methods).

FIG. 13: Sulfonate and sulfamate compounds. UV chromatogram (220-400 nm) of the LC/MS analysis of 100 μM of compounds (4a-4f) incubated with a reference (100 μM) in the PBS buffer at pH 8, 37° C. for four days. For compounds Sulfopin, 4a, and 4b, spectra at 220 nm were taken to show the peaks. Sulfopin and 4a underwent 30 and 15% hydrolysis, respectively. Compound 4a shows a Sulfopin-like peak, possibly due to displacement of sulfonate group by chloride ion in the buffer. “#” refers to hydrolysis product “*” refers to unidentified peak.

FIG. 14: Sulfamates compounds show high metabolic stability. Compounds (Sulfopin, 4d, 4e, and 4g) were incubated in liver microsomes and injected to LC/MS/MS at indicated time points. The peak intensities of the compounds were measured in UV spectra.

FIGS. 15A-15F: Molecular modeling of Sulfamate compounds. The models show that there is room to accommodate the sulfamate modification in the enzyme binding site next to the active pocket. In several of these models, both the sulfamate and the additional modification on the amine side make additional interactions with Pin1.

FIG. 16A-16C: Sulfamates compounds show cellular engagement with Pin1. FIG. 16A. Chemical structures of Sulfopin-DTB probe. FIG. 16B, 16C. Cellular engagement of the Sulfamates. OCI-AML2 cells were treated with DMSO, Sulfopin and sulfamate compounds (4c, 4d, 4e, 4g) at 0.5, 2.5 μM concentration for 4 hours. Lysates were prepared and incubated with Sulfopin DTB probe (1 μM) and then pulled down using streptavidin beads before running a western blot against Pin1.

FIG. 17A-17D: Sulfamate 4d show a five-fold higher binding affinity to Pin1 than sulfopin: FIGS. 17A and 17C show the depletion of unlabeled Pin1 (0.5 μM) when incubated with sulfopin (0.5, 1, 2, 5, 20, 50, 100, 150, 200 μM) or 4d (0.5, 1, 2, 5, 10, 20, 50, 100 μM) in 100 mM Tris buffer at pH 7.4, 14° C. over time. The reaction was monitored by LC-MS. FIGS. 17B and 17D. The rate constants as extrapolated from FIGS. 17A and 17C, respectively, as a function of the concentration of sulfopin and 4d. Kinetic parameters were obtained by fitting this data to a Michaelis-Menten saturation curve.

It will be appreciated that for simplicity and clarity of illustration, elements shown in the figures have not necessarily been drawn to scale. Further, where considered appropriate, reference numerals may be repeated among the figures to indicate corresponding or analogous elements.

### DETAILED DESCRIPTION

This invention is directed to pharmacology, and more particularly, but not exclusively, to newly designed compounds that covalently bind to, and/or modulate the activity of, Pin1 and to uses thereof in, for example, treating diseases associated with Pin1 activity.

### Sulfonate and Sulfamate Compounds

In some embodiments, this invention is directed to sulfonate and sulfamate-compounds represented by the structure of Formula IA:

E-L-G (Formula IA)

or a pharmaceutically acceptable salt thereof;
wherein,
G is a rigid moiety represented by the following formula (sulfopin derivative):

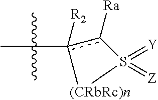

E is an electrophilic moiety represent by the following structure:

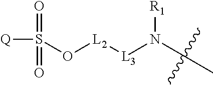

L is a bond or a linking moiety,
the [symbol] represents a saturated or non-saturated bond;
   Y and Z are each independently selected from the group consisting of O, S and NH;
   Q is selected from the group consisting of substituted or unsubstituted linear or branched alkyl, substituted or unsubstituted linear or branched alkyl, substituted or unsubstituted linear or branched alkenyl, substituted or unsubstituted linear or branched alkynyl, substituted or unsubstituted cycloalkyl, substituted or unsubstituted heterocyclic, substituted or unsubstituted aryl, substituted or unsubstituted heteroaryl and NR₃R₄;
   each of R₂, Ra, Rb and Rc are independently selected from the group consisting of hydrogen, alkyl, alkenyl, alkynyl, cycloalkyl, aryl, heteroaryl, heterocyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, or alternatively, R₂ is absent when the dashed line represents an unsaturated bond;
   R₁, R₃ and R₄ are each independently selected from the group consisting of hydrogen, OH, any group that forms a urea, an amide, a thiourea, hydrazide or hydroxamic acid (via the nitrogen), substituted or unsubstituted linear or branched alkyl, alkenyl, alkynyl, substituted or unsubstituted cycloalkyl, substituted or unsubstituted heterocyclyl, substituted or unsubstituted aryl, and substituted or unsubstituted heteroaryl, or R₃ and R₄ forms together a five or six membered ring with the nitrogen;
   n is 1, 2, 3 or 4;
   L₂ is substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group;
   L₃ is a bond or C═W, wherein W is O, S or NRs; and
   R₅ is selected from the group consisting of hydrogen, alkyl, alkenyl, alkynyl, cycloalkyl, heterocyclic, aryl and heteroaryl.

In another embodiment, the linking moiety represented by L may optionally be any linking group described herein and known in the art, optionally a hydrocarbon (as defined herein). In another embodiment, the linking moiety is substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group.

In some embodiments, this invention is directed to Sulfamate compound represented by the structure of Formula IB:

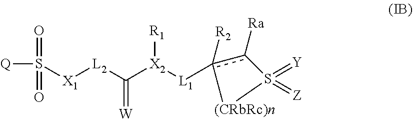

or a pharmaceutically acceptable salt thereof;
wherein,
the [symbol] represents a saturated or non-saturated bond;
Y and Z are each independently selected from the group consisting of O, S and NH;
X₁ and X₂ are each independently O, N, or CH;
Q is selected from the group consisting of substituted or unsubstituted linear or branched alkyl, substituted or unsubstituted linear or branched alkyl, substituted or unsubstituted linear or branched alkenyl, substituted or unsubstituted linear or branched alkynyl, substituted or unsubstituted cycloalkyl, substituted or unsubstituted heterocyclic, substituted or unsubstituted aryl, substituted or unsubstituted heteroaryl and NR₃R₄;
each of R₂ and Ra, Rb and Rc are independently selected from the group consisting of hydrogen, alkyl, alkenyl, alkynyl, cycloalkyl, aryl, heteroaryl, heterocyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, or alternatively, R₂ is absent when the dashed line represents an unsaturated bond;
each of R₁, R₃ and R₄ are independently selected from the group consisting of hydrogen, OH, any group that forms a urea, an amide, a thiourea, hydrazide or hydroxamic acid (via the nitrogen), substituted or unsubstituted linear or branched alkyl, alkenyl, alkynyl, substituted or unsubstituted cycloalkyl, substituted or unsubstituted heterocyclyl, substituted or unsubstituted aryl, and substituted or unsubstituted heteroaryl, or R₃ and R₄ forms together a five or six membered ring with the nitrogen; wherein when X₂ is O then R₁ is absent.
n is 1, 2, 3 or 4;
L₁ is a bond, substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group;
L₂ is substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group;
W is O, S or NRs; and
R₅ is selected from the group consisting of hydrogen, alkyl, alkenyl, alkynyl, cycloalkyl, heterocyclic, aryl and heteroaryl.

In some embodiments, this invention is directed to Sulfamate compound represented by the structure of Formula I.

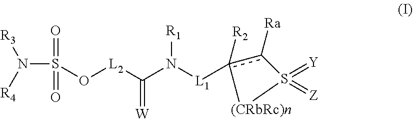

or a pharmaceutically acceptable salt thereof;
wherein,
the [symbol] represents a saturated or non-saturated bond;
Y and Z are each independently selected from the group consisting of O, S and NH;
each of R₂, Ra, Rb and Rc are independently selected from the group consisting of hydrogen, alkyl, alkenyl, alkynyl, cycloalkyl, aryl, heteroaryl, heterocyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, or alternatively, R₂ is absent when the dashed line represents an unsaturated bond;
each of R₁, R₃ and R₄ are independently selected from the group consisting of hydrogen, OH, any group that forms a urea, an amide, a thiourea, hydrazide or hydroxamic acid (via the nitrogen), substituted or unsubstituted linear or branched alkyl, alkenyl, alkynyl, substituted or unsubstituted cycloalkyl, substituted or unsubstituted heterocyclyl, substituted or unsubstituted aryl, and substituted or unsubstituted heteroaryl, or R₃ and R₄ forms together a five or six membered ring with the nitrogen;
n is 1, 2, 3 or 4;
L₁ is a bond, substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group;
L₂ is substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group;
W is O, S or NRs; and
R₅ is selected from the group consisting of hydrogen, alkyl, alkenyl, alkynyl, cycloalkyl, heterocyclic, aryl and heteroaryl.

In some embodiments, this invention is directed to Sulfamate compound represented by the structure of Formula II:

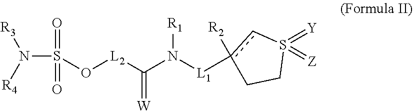

or a pharmaceutically acceptable salt thereof,
wherein,
the [symbol] represents a saturated or non-saturated bond;
Y and Z are each independently selected from the group consisting of O, S and NH;
R₂ is selected from the group consisting of hydrogen, alkyl, alkenyl, alkynyl, cycloalkyl, aryl, heteroaryl, heterocyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, or alternatively, R₂ is absent when the dashed line represents an unsaturated bond;
each of R₁, R₃ and R₄ are independently selected from the group consisting of hydrogen, OH, any group that forms a urea, an amide, a thiourea, hydrazide or hydroxamic acid (via the nitrogen), substituted or unsubstituted linear or branched alkyl, alkenyl, alkynyl, substituted or unsubstituted cycloalkyl, substituted or unsubstituted heterocyclyl, substituted or unsubstituted aryl, and substituted or unsubstituted heteroaryl, or R₃ and R₄ forms together a five or six membered ring with the nitrogen;
L₁ is a bond, substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group;
L₂ is substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group;
W is O, S or NR₅; and
R₅ is selected from the group consisting of hydrogen, alkyl, alkenyl, alkynyl, cycloalkyl, heterocyclic, aryl and heteroaryl.

In some embodiments, this invention is directed to Sulfamate compound represented by the structure of Formula III:

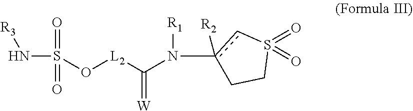

or a pharmaceutically acceptable salt thereof;
wherein,
   the [symbol] represents a saturated or non-saturated bond;
   R₂ is selected from the group consisting of hydrogen, alkyl, alkenyl, alkynyl, cycloalkyl, aryl, heteroaryl, heterocyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, or alternatively, R₂ is absent when the dashed line represents an unsaturated bond;
   each of R₁ and R₃ are independently selected from the group consisting of hydrogen, OH, any group that forms a urea, an amide, a thiourea, hydrazide or hydroxamic acid (via the nitrogen), substituted or unsubstituted linear or branched alkyl, alkenyl, alkynyl, substituted or unsubstituted cycloalkyl, substituted or unsubstituted heterocyclyl, substituted or unsubstituted aryl, and substituted or unsubstituted heteroaryl;
L₂ is substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group;
W is O, S or NRs; and
R₅ is selected from the group consisting of hydrogen, alkyl, alkenyl, alkynyl, cycloalkyl, heterocyclic, aryl and heteroaryl.

In some embodiments, this invention is directed to Sulfamate compound represented by the structure of Formula IV:

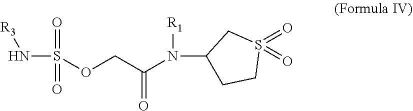

or a pharmaceutically acceptable salt thereof,
wherein,
   each of R₁ and R₃ are independently selected from the group consisting of hydrogen, OH, any group that forms a urea, an amide, a thiourea, hydrazide or hydroxamic acid (via the nitrogen), substituted or unsubstituted linear or branched alkyl, alkenyl, alkynyl, substituted or unsubstituted cycloalkyl, substituted or unsubstituted heterocyclyl, substituted or unsubstituted aryl, and substituted or unsubstituted heteroaryl.

In some embodiments of the compounds represented by Formula (IA), (IB), (I), (II), and (III), [symbol] represents a single bond or a double bond. In some embodiments of the compounds represented by Formula (IA), (IB), (I), (II), and (III), [symbol] represents a single bond. In some embodiments of the compounds represented by Formula (IA), (IB), (I), (II), and (III), [symbol] represents a double bond.

In some embodiments, R₁ of the compound represented by Formula IA, IB, I, II, III, or IV is selected from the group consisting of hydrogen, substituted or unsubstituted linear or branched alkyl, alkenyl, alkynyl, substituted or unsubstituted cycloalkyl, substituted or unsubstituted heterocyclyl, substituted or unsubstituted aryl, or substituted or unsubstituted heteroaryl. In another embodiment, R₁ is hydrogen. In another embodiment, R₁ is substituted or unsubstituted linear or branched alkyl. In another embodiment, R₁ is C₁-C₆ alkyl which is unsubstituted or substituted by C₄-C₇ cycloalkyl, halo, hydroxy, alkoxy, cyano, or oxo. In another embodiment, R₁ is C₁-C₆ alkyl which is unsubstituted or substituted by C₅-C₆ cycloalkyl, halo, hydroxy, alkoxy, cyano, or oxo. In another embodiment, R₁ is unsubstituted C₅-C₆ alkyl. In another embodiment, R₁ is C₁-C₃ alkyl which is unsubstituted or substituted by C₅-C₆ cycloalkyl, halo, hydroxy, alkoxy, cyano, or oxo. In another embodiment, R₁ is C₁-C₂ alkyl which is unsubstituted or substituted by C₅-C₆ cycloalkyl, halo, hydroxy, alkoxy, cyano, or oxo. In another embodiment, R₁ is C₁-C₂ alkyl which is substituted by C₅-C₆ cycloalkyl, halo, hydroxy, alkoxy, cyano, or oxo. In another embodiment, R₁ is unsubstituted C₅-C₆ alkyl or C₁-C₂ alkyl which is substituted by C₅-C₆ cycloalkyl, halo, hydroxy, alkoxy, cyano, or oxo. In another embodiment, R₁ is —CH₂—C(CH₃)₃. In another embodiment, R₁ is —CH₂—CH(CH₃)₂. In another embodiment, R₁ is alkenyl. In another embodiment, R₁ is alkynyl. In another embodiment, R₁ is substituted or unsubstituted cycloalkyl. In another embodiment, R₁ is substituted or unsubstituted heterocyclyl. In another embodiment, R₁ is substituted or unsubstituted aryl. In another embodiment, R₁ is substituted or unsubstituted heteroaryl.

In another embodiment, R₁ of the compound represented by Formula IA, IB, I, II, III, or IV is substituted or unsubstituted linear alkyl represented by Formula A:

—CH₂-Q′ (Formula A),

wherein Q′ is selected from the group consisting of alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino. Each represent a separate embodiment of this invention. In another embodiment, Q′ is tertiary alkyl, alkenyl, alkynyl, cycloalkyl or heterocyclic. In another embodiment Q′ is heteroaryl. In another embodiment Q′ is a substituted or unsubstituted t-butyl, or substituted or unsubstituted cycloalkyl. In another embodiment, Q′ is C₄-C₆ alkyl or C₅-C₇ cycloalkyl, each of which is unsubstituted or substituted by halo, hydroxy, alkoxy, cyano, or oxo. In another embodiment, Q′ is secondary or tertiary C₄-C₆ alkyl, or C₅-C₇ cycloalkyl, each of which is unsubstituted or substituted by halo, hydroxy, alkoxy, cyano, or oxo. In another embodiment, Q′ is tertiary C₄-C₆ alkyl, or C₅-C₇ cycloalkyl, each of which is unsubstituted or substituted by halo, hydroxy, alkoxy, cyano, or oxo. In another embodiment Q′ is tert butyl. In another embodiment Q′ is cyclohexyl.

According to some of any of the embodiments described herein relating to Formula IA, IB, I, II, III or IV Q′ is a tertiary alkyl, alkenyl, alkynyl, cycloalkyl, heterocycloalkyl, or heteroaryl.

In another embodiment, R₁ or Q′ is heteroaryl, wherein the heteroaryl is a triazole.

According to some of any of the embodiments described herein relating to a triazole, the triazole has Formula B:

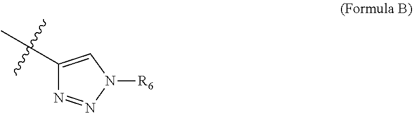

wherein R₆ is selected from the group consisting of alkyl, alkenyl, alkynyl, cycloalkyl, heteroalicyclic, aryl and heteroaryl. Each represent a separate embodiment of this invention.

In another embodiment, R₆ of Formula B is a substituted or unsubstituted phenyl. In another embodiment, R₆ is a phenyl substituted by a substituent selected from hydroxy, hydroxyalkyl, halo, alkoxy, carbonyl, carboxy or sulfonamido. In another embodiment, R₆ is p-methoxycarbonylphenyl.

In some embodiments, the dashed line of compound of Formula IA, IB, I, II or III represents a saturated bond. In some embodiments, the dashed line of compound of Formula IA, IB, I, II or III represents a non-saturated bond.

In some embodiments, R₂ of the compound represented by Formula IA, IB, I, II, III, or IV is selected from the group consisting of hydrogen, alkyl, alkenyl, alkynyl, cycloalkyl, aryl, heteroaryl, heterocyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, or alternatively, R₂ is absent when the dashed line represents an unsaturated bond. Each represent a separate embodiment of this invention. In another embodiment, R₂ is hydrogen. In another embodiment, R₂ is C₁-C₁₀ alkyl. In another embodiment, R₂ is C₁-C₆ alkyl. In another embodiment, R₂ is H or C₁-C₆ alkyl. In another embodiment, R₂ is C₁-C₃ alkyl. In another embodiment, R₂ is H or C₁-C₃ alkyl.

In some embodiments, R₃ of the compound of Formula IA, IB, I, II, III, or IV is selected from the group consisting of hydrogen, OH, any group that forms a urea, an amide, a thiourea, hydrazide or hydroxamic acid via the nitrogen, substituted or unsubstituted linear or branched alkyl, alkenyl, alkynyl, substituted or unsubstituted cycloalkyl, substituted or unsubstituted heterocyclyl, substituted or unsubstituted aryl, and substituted or unsubstituted heteroaryl. Each represent a separate embodiment of this invention. In some embodiments, R₃ is selected from the group consisting of C₁-C₁₀ alkyl, benzyl, or phenyl, wherein the C₁-C₁₀ alkyl of R₃ is unsubstituted or substituted with halo, hydroxy, alkoxy, cyano, or oxo; and the benzyl or phenyl of R₃ is unsubstituted or substituted with halo, alkyl, hydroxy, alkoxy, or cyano. In some embodiments, R₃ is selected from the group consisting of unsubstituted C₁-C₁₀ alkyl, unsubstituted benzyl, or phenyl, wherein the phenyl of R₃ is unsubstituted or substituted with halo or C₁-C₃ alkyl. In some embodiments, R₃ is selected from the group consisting of C₁-C₃ alkyl, benzyl, or phenyl, wherein the C₁-C₃ alkyl of R₃ is unsubstituted or substituted with halo, hydroxy, alkoxy, cyano, or oxo; and the benzyl or phenyl of R₃ is unsubstituted or substituted with halo, alkyl, hydroxy, alkoxy, or cyano. In some embodiments, R₃ is selected from the group consisting of unsubstituted C₁-C₃ alkyl, unsubstituted benzyl, or phenyl, wherein the phenyl of R₃ is unsubstituted or substituted with halo or C₁-C₃ alkyl. In another embodiment, R₃ is C₁-C₁₀ alkyl. In another embodiment, R₃ is methyl. In another embodiment, R₃ is substituted or unsubstituted benzyl. In another embodiment, R₃ is benzyl. In another embodiment, R₃ is substituted or unsubstituted aryl. In another embodiment, R₃ is substituted or unsubstituted phenyl. In another embodiment, R₃ is halo substituted phenyl. In another embodiment, the halo is selected from I, Br, Cl or F. In another embodiment, R₃ is 4-bromo-phenyl. In another embodiment, R₃ is alkyl substituted phenyl. In another embodiment, R₃ is 4-methyl-phenyl. In another embodiment, R₃ is OH. In another embodiment, R₃ is any group that forms a urea, an amide, a thiourea, hydrazide or hydroxamic acid (via the nitrogen). In some embodiments, “any group that forms a urea” indicates that R₃ is C(O)NRₓR_{y}; “any group that forms an amide” indicates that R₃ is C(O)R_{y}; and “any group that forms a thiourea” indicates that R₃ is C(S)NRₓR_{y}. In some embodiments, “any group that forms a hydrazide” indicates that R₃ is NRₓC(O)R_{y}, or that R₃ is C(O)R_{y} and R₄ is NRₓR_{y}. In some embodiments, “any group that forms a hydroxamic acid” indicates that R₃ is C(O)R_{y} and R₄ is OH. In some embodiments of the foregoing, each Rₓ is independently H or is selected from C₁-C₆ alkyl or C₄-C₇ cycloalkyl, each of which is unsubstituted or substituted by halo, hydroxy, alkoxy, cyano, or oxo; and each R_{y} is independently selected from C₁-C₆ alkyl or C₄-C₇ cycloalkyl, each of which is unsubstituted or substituted by halo, hydroxy, alkoxy, cyano, or oxo. In some embodiments, R₃ of the compound of Formula IA, IB, I, II, III or IV is selected from the group consisting of hydrogen, unsubstituted C₁-C₁₀ alkyl, substituted C₁-C₁₀ alkyl, unsubstituted aryl, and substituted aryl. In some embodiments, R₃ of the compound of Formula IA, IB, I, II, III, or IV is selected from the group consisting of hydrogen, unsubstituted C₁-C₆ alkyl, substituted C₁-C₆ alkyl, unsubstituted phenyl, and substituted phenyl. In some embodiments, R₃ of the compound of Formula IA, IB, I, II, III, or IV is selected from the group consisting of hydrogen, unsubstituted C₁-C₆ alkyl, substituted C₁-C₆ alkyl, unsubstituted phenyl, and substituted phenyl. In some embodiments, R₃ of the compound of Formula IA, IB I, II, III, or IV is selected from the group consisting of hydrogen, unsubstituted C₁-C₃ alkyl, benzyl, unsubstituted phenyl, and phenyl substituted with halo or C₁-C₃ alkyl.

In some embodiments, R₄ of the compound of Formula IA, IB, I, or II is selected from the group consisting of hydrogen, substituted or unsubstituted linear or branched alkyl, alkenyl, alkynyl, substituted or unsubstituted cycloalkyl, substituted or unsubstituted heterocyclyl, substituted or unsubstituted aryl, or substituted or unsubstituted heteroaryl. Each represent a separate embodiment of this invention. In another embodiment, R₄ is a hydrogen. In another embodiment, R₄ is C₁-C₁₀ alkyl. In another embodiment, R₄ is methyl. In another embodiment, R₄ is substituted or substituted benzyl. In another embodiment, R₄ is benzyl. In another embodiment, R₄ is substituted or substituted aryl. In another embodiment, R₄ is substituted or substituted benzyl. In another embodiment, R₄ is benzyl. In another embodiment, R₄ is substituted or unsubstituted phenyl. In another embodiment, R₄ is halo substituted phenyl. In another embodiment, R₄ is 4-bromo-phenyl. In another embodiment, R₄ is alkyl substituted phenyl. In another embodiment, R₄ is 4-methyl-phenyl. In another embodiment, R₄ is OH. In another embodiment, R₄ is any group that forms a urea, an amide, a thiourea, hydrazide or hydroxamic acid (via the nitrogen). In some embodiments, R₄ of the compound of Formula IA, IB, I, II, III or IV is selected from the group consisting of hydrogen, unsubstituted C₁-C₁₀ alkyl, substituted C₁-C₁₀ alkyl, substituted or unsubstituted benzyl, unsubstituted aryl, and substituted aryl. In some embodiments, R₄ of the compound of Formula IA, IB, I, II, III or IV is selected from the group consisting of hydrogen, unsubstituted C₁-C₆ alkyl, substituted C₁-C₆ alkyl, unsubstituted phenyl, and substituted phenyl. In some embodiments, R₄ of the compound of Formula IA, IB, I, II, III or IV is selected from the group consisting of hydrogen, unsubstituted C₁-C₆ alkyl, substituted C₁-C₆ alkyl, substituted or unsubstituted benzyl, unsubstituted phenyl, and substituted phenyl. In some embodiments, R₄ of the compound of Formula IA, IB I, II, III or IV is selected from the group consisting of hydrogen, unsubstituted C₁-C₃ alkyl, benzyl, unsubstituted phenyl, and phenyl substituted with halo or C₁-C₃ alkyl.

In some embodiments, R₃ of the compound of Formula IA, IB, I, or II is selected from the group consisting of hydrogen, unsubstituted C₁-C₁₀ alkyl, substituted C₁-C₁₀ alkyl, unsubstituted aryl, and substituted aryl; and R₄ is hydrogen. In some embodiments, R₃ of the compound of Formula IA, IB, I, or II is selected from the group consisting of hydrogen, unsubstituted C₁-C₆ alkyl, substituted C₁-C₆ alkyl, unsubstituted phenyl, and substituted phenyl; and R₄ is hydrogen. In some embodiments, R₃ of the compound of Formula IA, IB, I, or II is selected from the group consisting of hydrogen, unsubstituted C₁-C₆ alkyl, substituted C₁-C₆ alkyl, unsubstituted phenyl, and substituted phenyl; and R₄ is hydrogen. In some embodiments, R₃ of the compound of Formula IA, IB, I, or II is selected from the group consisting of hydrogen, unsubstituted C₁-C₃ alkyl, benzyl, unsubstituted phenyl, and phenyl substituted with halo or C₁-C₃ alkyl; and R₄ is hydrogen.

In some embodiments, R₃ of the compound of Formula III or Formula IV is selected from the group consisting of C₁-C₁₀ alkyl, benzyl, or phenyl, wherein the C₁-C₁₀ alkyl of R₃ is unsubstituted or substituted with halo, hydroxy, alkoxy, cyano, or oxo; and the benzyl or phenyl of R₃ is unsubstituted or substituted with halo, alkyl, hydroxy, alkoxy, or cyano; and R₁ is of Formula A. In some embodiments, R₃ of the compound of Formula III or Formula IV is selected from the group consisting of C₁-C₁₀ alkyl, benzyl, or phenyl, wherein the C₁-C₁₀ alkyl of R₃ is unsubstituted or substituted with halo, hydroxy, alkoxy, cyano, or oxo; and the benzyl or phenyl of R₃ is unsubstituted or substituted with halo, alkyl, hydroxy, alkoxy, or cyano; and R₁ is of Formula A wherein Q′ is tertiary C₄-C₆ alkyl, or C₅-C₇ cycloalkyl, each of which is unsubstituted or substituted by halo, hydroxy, alkoxy, cyano, or oxo. In some embodiments, R₃ of the compound of Formula III or Formula IV is selected from the group consisting of C₁-C₁₀ alkyl, benzyl, or phenyl, wherein the C₁-C₁₀ alkyl of R₃ is unsubstituted or substituted with halo, hydroxy, alkoxy, cyano, or oxo; and the benzyl or phenyl of R₃ is unsubstituted or substituted with halo, alkyl, hydroxy, alkoxy, or cyano; and R₁ is of Formula A wherein Q′ is tert-butyl or cyclohexyl. In some such embodiments, Q′ is tert-butyl.

In some embodiments, R₃ of the compound of Formula III or Formula IV is selected from the group consisting of C₁-C₃ alkyl, benzyl, or phenyl, wherein the C₁-C₃ alkyl of R₃ is unsubstituted or substituted with halo, hydroxy, alkoxy, cyano, or oxo; and the benzyl or phenyl of R₃ is unsubstituted or substituted with halo, alkyl, hydroxy, alkoxy, or cyano; and R₁ is of Formula A. In some embodiments, R₃ of the compound of Formula III or Formula IV is selected from the group consisting of C₁-C₃ alkyl, benzyl, or phenyl, wherein the C₁-C₃ alkyl of R₃ is unsubstituted or substituted with halo, hydroxy, alkoxy, cyano, or oxo; and the benzyl or phenyl of R₃ is unsubstituted or substituted with halo, alkyl, hydroxy, alkoxy, or cyano; and R₁ is of Formula A, wherein Q′ is tertiary C₄-C₆ alkyl, or C₅-C₇ cycloalkyl, each of which is unsubstituted or substituted by halo, hydroxy, alkoxy, cyano, or oxo. In some embodiments, R₃ of the compound of Formula III or Formula IV is selected from the group consisting of C₁-C₃ alkyl, benzyl, or phenyl, wherein the C₁-C₃ alkyl of R₃ is unsubstituted or substituted with halo, hydroxy, alkoxy, cyano, or oxo; and the benzyl or phenyl of R₃ is unsubstituted or substituted with halo, alkyl, hydroxy, alkoxy, or cyano; and R₁ is of Formula A, wherein Q′ is tert-butyl or cyclohexyl. In some such embodiments, Q′ is tert-butyl.

In some embodiments, R₃ of the compound of Formula III or Formula IV is selected from the group consisting of unsubstituted C₁-C₃ alkyl, unsubstituted benzyl, or phenyl, wherein the phenyl of R₃ is unsubstituted or substituted with halo, C₁-C₃ alkyl, hydroxy, alkoxy, or cyano; and R₁ is of Formula A, wherein Q′ is tertiary C₄-C₆ alkyl, or C₅-C₇ cycloalkyl, each of which is unsubstituted or substituted by halo, hydroxy, alkoxy, cyano, or oxo. In some embodiments, R₃ of the compound of Formula III or Formula IV is selected from the group consisting of unsubstituted C₁-C₃ alkyl, unsubstituted benzyl, or phenyl, wherein the phenyl of R₃ is unsubstituted or substituted with halo, C₁-C₃ alkyl, hydroxy, alkoxy, or cyano; and R₁ is of Formula A, wherein Q′ is tert-butyl or cyclohexyl. In some such embodiments, Q′ is tert-butyl.

In some embodiments, R₃ of the compound of Formula III or Formula IV is selected from the group consisting of unsubstituted C₁-C₃ alkyl, unsubstituted benzyl, or phenyl, wherein the phenyl of R₃ is unsubstituted or substituted with halo or C₁-C₃ alkyl; and R₁ is of Formula A, wherein Q′ is tertiary C₄-C₆ alkyl, or C₅-C₇ cycloalkyl, each of which is unsubstituted or substituted by halo, hydroxy, alkoxy, cyano, or oxo. In some embodiments, R₃ of the compound of Formula III or Formula IV is selected from the group consisting of unsubstituted C₁-C₃ alkyl, unsubstituted benzyl, or phenyl, wherein the phenyl of R₃ is unsubstituted or substituted with halo or C₁-C₃ alkyl; and R₁ is of Formula A, wherein Q′ is tert-butyl or cyclohexyl. In some such embodiments, Q′ is tert-butyl.

In some embodiments R₃ and R₄ of formula IA, IB, I or II form together a five or six membered ring together with the nitrogen. The ring can be substituted or unsubstituted. Non limited rings include: pyridine, piperidine, pyperazine, morpholine, pyrrolidine or pyrimidine.

In some embodiments, L₁ of the compound of Formula IB, I, or II, is a bond, substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group. Each represent a separate embodiment of this invention. In another embodiment, L₁ is a bond.

In some embodiments, L₂ of the compound of Formula IA, IB, I, II, or III is substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group. Each represent a separate embodiment of this invention. In another embodiment, L₂ is C₁-C₁₀ alkylene. In another embodiment, L₂ is C₁-C₅ alkylene. In another embodiment, L₂ is C₁-C₃ alkylene. In another embodiment L₂ is methylene.

In some embodiments, W of the compound of Formula IA, IB, I, II, or III is O, S or NRs. Each represent a separate embodiment of this invention. In another embodiment, W is O. In another embodiment, W is S. In another embodiment, W is NR₅. In another embodiment, R wherein W is NR₅ is selected from the group consisting of hydrogen, alkyl, alkenyl, alkynyl. Each represent a separate embodiment of this invention.

In some embodiments, Y and Z of the compound of Formula IA, IB, I, or II are each independently selected from the group consisting of O, S and NH. Each represent a separate embodiment of this invention. In some embodiments, Y and Z are each independently selected from the group consisting of O and NH. In some embodiments, Y is O and Z is O or NH. In some embodiments, Y is O and Z is O. In another embodiment, Y is O. In another embodiment, Z is O. In some of any of the respective embodiments described herein, Y and Z are each oxygen, thus forming a cyclic sulfone. In some such embodiments, n is 2 such that the cyclic sulfone is a sulfolane or sulfolene. In some such embodiments, n is 2 such that the cyclic sulfone is a sulfolane.

In some embodiments, Ra, Rb and Rc of the compound of Formula IA, IB, or I are each independently selected from the group consisting of hydrogen, alkyl, alkenyl, alkynyl, cycloalkyl, aryl, heteroaryl, heterocyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino. Each represent a separate embodiment of this invention. In another embodiments, Ra is hydrogen. In another embodiments, Rb is hydrogen. In another embodiment, Rb and Rc are each hydrogen. In some embodiment, Ra, Rb and Rc are each hydrogen.

In some embodiments, n of the compound of Formula IA, 1, or I is an integer between 1-4. In another embodiment, n is 1. In another embodiment, n is 2. In another embodiment, n is 3. In another embodiment, n is 4.

In some embodiments, X₁ and X₂ of the compound of Formula TB are each independently O, N, or CH. Each represent a separate embodiment of this invention. In another embodiment, X₁ is O. In another embodiment, X₁ is N. In another embodiment, X₁ is CHI. In another embodiment, X₂ is N. In another embodiment, X₂ is O. In another embodiment, X₂ is CHI. In another embodiment, if X₂ is O, then R₁ is nothing/absent.

In some embodiments, the compound of this invention is presented by the structures of Formula IA, IB, I, II, III, IV, or a pharmaceutically acceptable salt thereof.

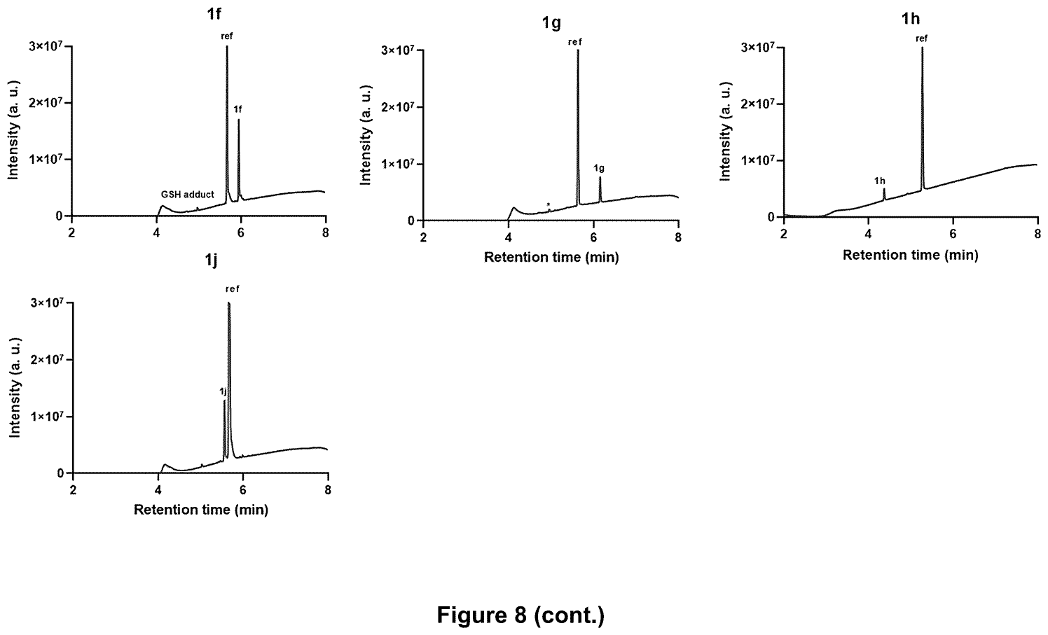

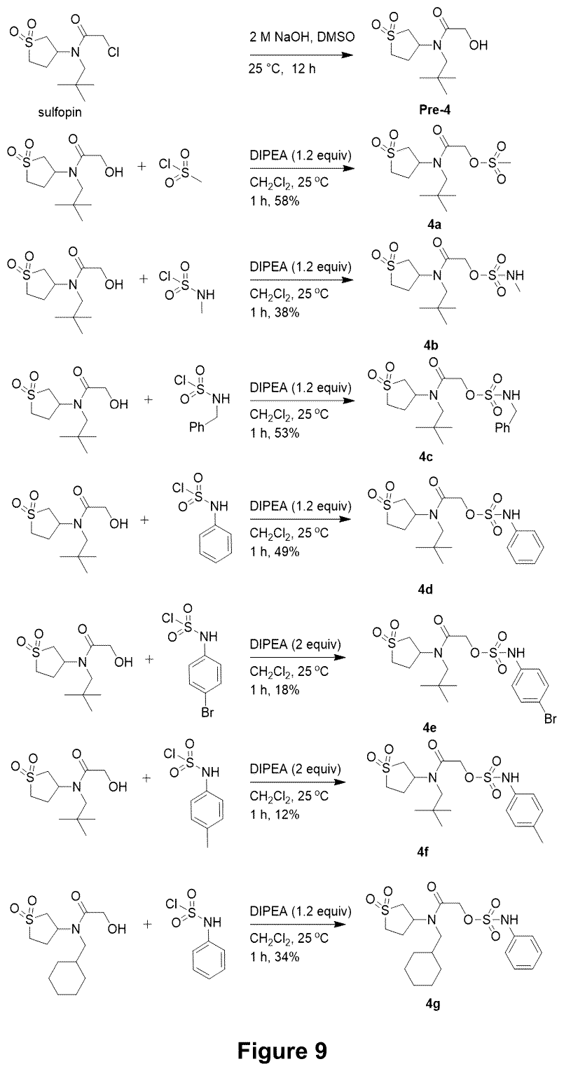

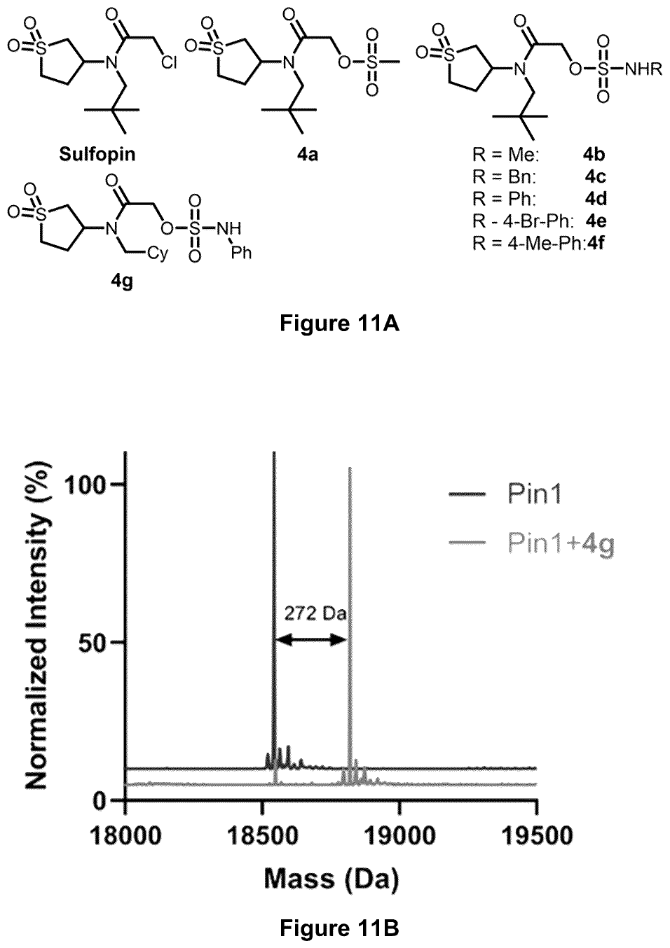

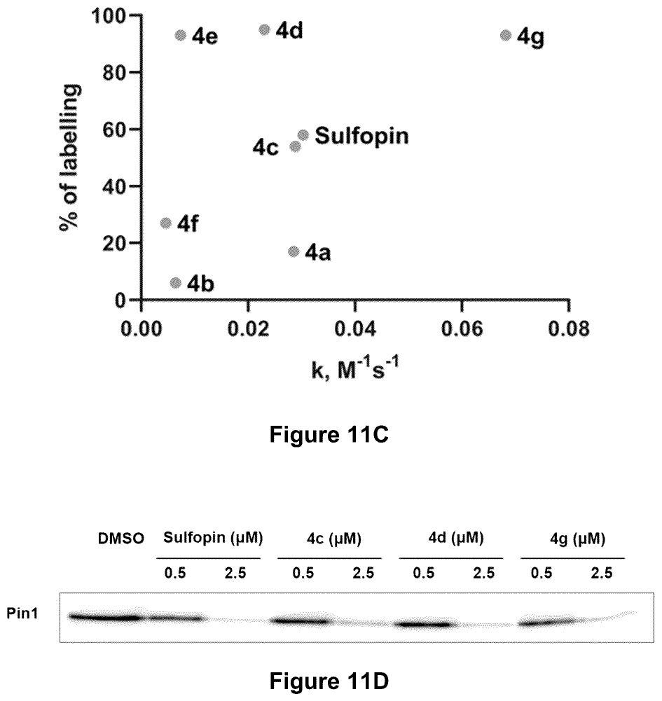

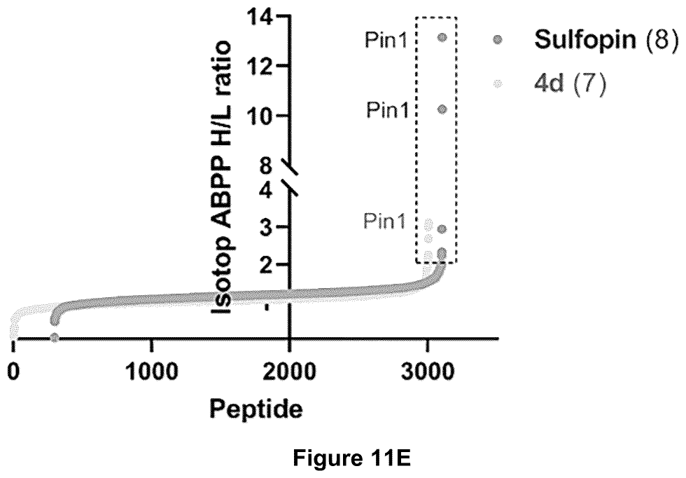

In some embodiments, exemplified compounds of the compounds of Formula IA, IB, I, II, III and IV of this invention are represented in Table 1 (FIGS. 9 and 11A):

TABLE 1
Exemplified compounds of the compounds of
Formula IA, IB, I, II, III and IV.

| Compound | |
|---|---|
| number | Structure |
| 4b | 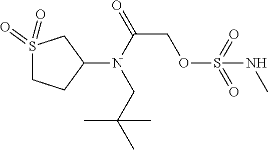 [SMILES: CNS(=O)(=O)OCC(=O)N(CC(C)(C)C)C1CCS(=O)(=O)C1] |
| 4c | 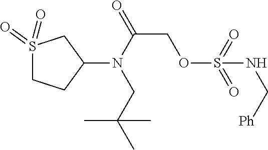 [SMILES: CC(C)(C)CN(C(=O)COS(=O)(=O)NCc1ccccc1)C1CCS(=O)(=O)C1] |
| 4d | 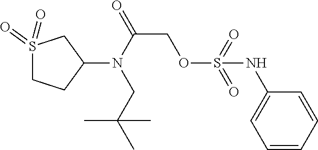 [SMILES: CC(C)(C)CN(C(=O)COS(=O)(=O)Nc1ccccc1)C1CCS(=O)(=O)C1] |
| 4e | 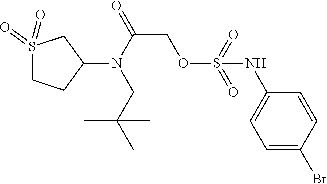 [SMILES: CC(C)(C)CN(C(=O)COS(=O)(=O)Nc1ccc(Br)cc1)C1CCS(=O)(=O)C1] |
| 4f | 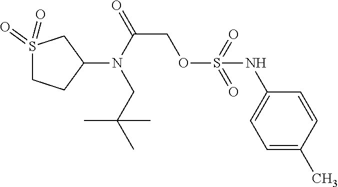 [SMILES: Cc1ccc(NS(=O)(=O)OCC(=O)N(CC(C)(C)C)C2CCS(=O)(=O)C2)cc1] |
| 4g | 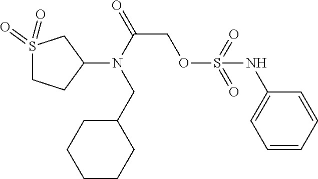 [SMILES: O=C(COS(=O)(=O)Nc1ccccc1)N(CC1CCCCC1)C1CCS(=O)(=O)C1] |

### Synthesis of Sulfamate Compounds

In some embodiments, provided herein is a synthesis of Sulfamate compounds of this invention, as shown in scheme 1:

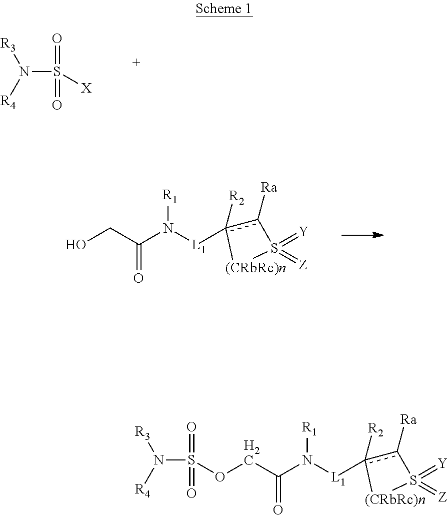

wherein,
X is a leaving group;
the [symbol] represents saturated or non-saturated bond;
Y and Z are each independently selected from the group consisting of O, S and NH;
each of R₂, Ra, Rb and Rc are independently selected from the group consisting of hydrogen, alkyl, alkenyl, alkynyl, cycloalkyl, aryl, heteroaryl, heterocyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, or alternatively, R₂ is absent when the dashed line represents an unsaturated bond;
each of R₁, R₃ and R₄ are independently selected from the group consisting of hydrogen, OH, any group that forms a urea, an amide, a thiourea, hydrazide or hydroxamic acid (via the nitrogen), substituted or unsubstituted linear or branched alkyl, alkenyl, alkynyl, substituted or unsubstituted cycloalkyl, substituted or unsubstituted heterocyclyl, substituted or unsubstituted aryl, and substituted or unsubstituted heteroaryl;
n is 1, 2, 3 or 4; and
L₁ is a bond, substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group.

In some embodiments, DIPEA (N,N-diisopropylethylamine) is added to the reaction.

In some embodiments, X is a halo group. In another embodiment, X is Cl. In another embodiment, X is Br. In another embodiment, X is F. In another embodiment, X is I.

### Modulation of the Activity of Pint

According to some embodiments of the present invention, a compound as described herein has high affinity to the catalytic binding site of Pin1.

In some embodiments, there is provided a compound for use in modulating an activity of Pin1, the compound comprising an electrophilic moiety (E) and rigid moiety (G), wherein the electrophilic moiety and the rigid moiety are arranged such that the electrophilic moiety is capable of covalently binding to the Cys113 residue of Pin1, and the rigid moiety is capable of forming hydrogen bonds with the Gln131 and His 157 residues of Pin1; and the sulfamate group forms hydrogen bonds with Pin1.

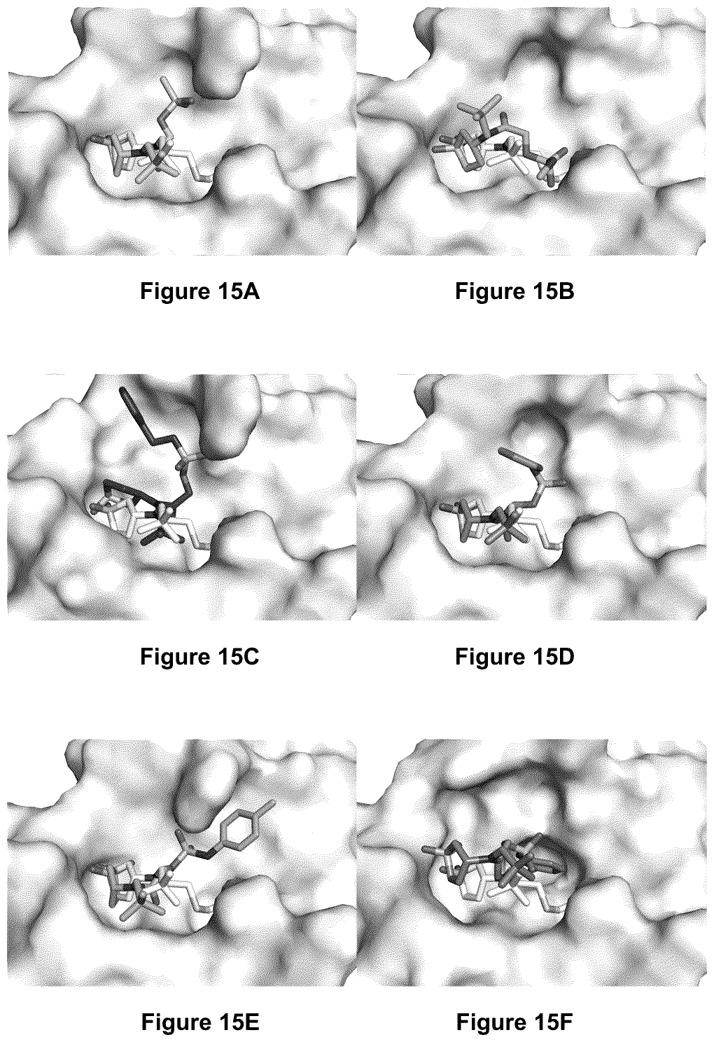

In another embodiment, the sulfamate group forms additional hydrogen bonds with protein Pin1 (according to the modeling in FIG. 15), and the sulfamate side chain has room to propagate into an additional pocket on the protein that mediates extra recognition (additional selectivity and tighter binder affinity). In another embodiment, the compounds of this invention have lower thiol reactivity than Sulfopin.

In some embodiments, the sulfamate compounds of this invention show advantages over conventional electrophiles such as acrylamides and chloroacetamides in terms of tunability of its intrinsic thiol reactivity, amino acid selectivity, and buffer/metabolic stability. Further, acrylamides, including ibrutinib[Scheers, E.; Leclercq, L.; de Jong, J.; Bode, N.; Bockx, M.; Laenen, A.; Cuyckens, F.; Skee, D.; Murphy, J.; Sukbuntherng, J.; Mannens, G. Absorption, Metabolism, and Excretion of Oral 14C Radiolabeled Ibrutinib: An Open-Label, Phase I, Single-Dose Study in Healthy Drug Metab. Dispos. 2015, 43 (2), 289-297, which is incorporated herein by reference], are oxidatively metabolized to epoxides, which are extremely reactive. Although such epoxides are rapidly destroyed by hydrolysis or GSH conjugation, they could potentially react with proteins resulting in haptenization. [Tang, L. W. T.; Fu, J.; Koh, S. K.; Wu, G.; Zhou, L.; Chan, E. C. Y. Drug Metabolism and Disposition. 2022, pp 931-941. https.//doi.org/, which is incorporated herein by reference]. By contrast, it seems plausible that sulfamate-acetamides would not be converted into even more reactive metabolites. In some embodiments, the sulfamic acid leaving group can self-immolatively dissociate into an amine functionality with the release of sulfur trioxide allowing further functionalization.

In some embodiment, the rigid moiety is capable of forming a hydrogen bond with a backbone amide hydrogen of the Gln131 and/or with an imidazole NH of the His157 or Pin1.

In some embodiments, the rigid moiety is or comprises a sulfolane or a sulfolene.

According to some of any of the embodiments described herein, the compound further comprises a hydrophobic moiety. In some embodiments, R₁ of Formula IA, IB, I, II, III and IV is a hydrophobic moiety.

According to some of any of the embodiments described herein relating to a hydrophobic moiety, the hydrophobic moiety forms a hydrophobic interaction with Ser115, Leu122 and/or Met130 of Pin1.

According to an aspect of some embodiments of the invention, there is provided a method of modulating an activity of Pin1, the method comprising contacting the Pin1 with a compound according to any of the respective embodiments described herein.

In some embodiments, the compounds of this invention of Formula IA, IB, I, II, III, IV or a pharmaceutically acceptable salt thereof are suitable for in vivo covalent targeting. In some embodiments, the compounds of this invention are suitable for in vivo covalent targeting of Pin 1.

In some embodiments, the Sulfamate compound for use as modulating Pin1 forms a covalent bond between the Cys113 residue of Pin1 and a carbon near the sulfamate group (electrophilic site), and releases a sulfamic acid leaving group; and hydrogen bonds are formed between Gln131 and His 157 residues of Pin1 and the sulfolane ring; and between protein Pin1 and the sulfamate side-chain.

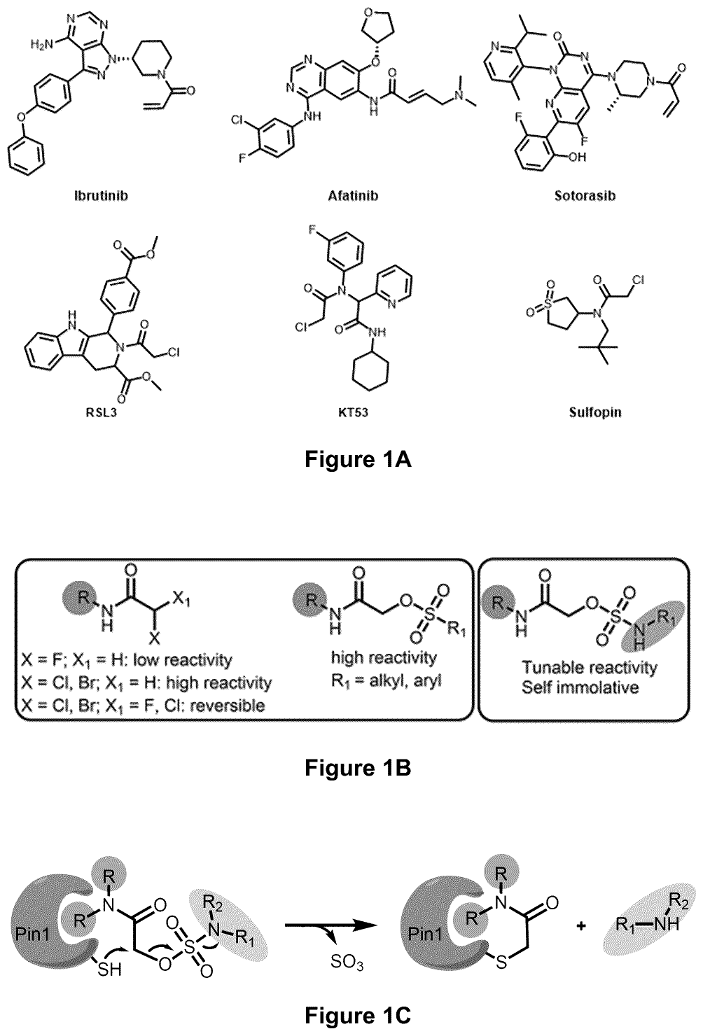

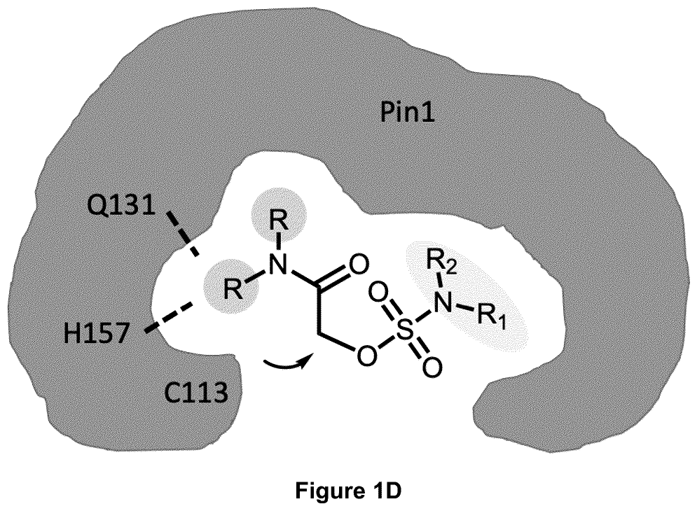

In some embodiments, the additional side-chain of the sulfamate can mediate additional interactions with the protein and improve the reversible affinity. (FIG. 1D)

In another embodiment, said sulfamic acid leaving group will dissociate into sulfur trioxide and free amine [Benson, G. A., Anthony Benson, G. & Spillane, W. J. Chemical Reviews vol. 80 151-186 (1980), which is incorporated herein by reference].

In some embodiments, the present invention refers to compounds of Formula IA, IB, I, II, III, IV or a pharmaceutically acceptable salt thereof, selective modulators of Pin1 activity, wherein the compounds of this invention are covalent inhibitors of Pin1. In some embodiments, the compound of this invention of Formula IA, IB, I, II, III, IV, 4a-4g, or a pharmaceutically acceptable salt thereof is selective covalent active site (catalytic domain) inhibitor of Pin1, as well as the effects of selective modulation of Pin1 activity in various physiological models.

As used herein, the phrase “catalytic domain” describes a region of an enzyme, Pin1, in which the catalytic reaction occurs. This phrase therefore describes this part of an enzyme in which the substrate and/or other components that participate in the catalytic reaction interacts with the enzyme. In the context of the present embodiments, this phrase is particularly used to describe this part of an enzyme (a Pin1) to which the substrate binds during the catalytic activity (e.g., phosphorylation). This phrase is therefore also referred to herein and in the art, interchangeably, as “substrate binding pocket”, “catalytic site” “active site” and the like.

As used herein, the phrases “binding site”, “catalytic binding site” or “binding subsite”, which are used herein interchangeably, describe a specific site in the catalytic domain that includes one or more reactive groups through which the interactions of the enzyme with the substrate and/or an inhibitor can be effected. Typically, the binding site is composed of one or two amino acid residues, whereby the interactions typically involve reactive groups at the side chains of these amino acids.

As is well known in the art, when an enzyme interacts with a substrate or an inhibitor, the initial interaction rapidly induces conformational changes, in the enzyme and/or substrate and/or inhibitor, that strengthen binding and bring enzyme's binding sites close to functional groups in the substrate or inhibitor. Enzyme-substrate/inhibitor interactions orient reactive groups present in both the enzyme and the substrate/inhibitor and bring them into proximity with one another. The binding of the substrate/inhibitor to the enzyme aligns the reactive groups so that the relevant molecular orbitals overlap.

Thus, an inhibitor of an enzyme is typically associated with the catalytic domain of the enzyme such that the reactive groups of the inhibitor are positioned in sufficient proximity to corresponding reactive groups (typically side chains of amino acid residues) in the enzyme catalytic binding site, so as to allow the presence of an effective concentration of the inhibitor in the catalytic binding site and, in addition, the reactive groups of the inhibitor are positioned in a proper orientation, to allow overlap and thus a strong chemical interaction and low dissociation. An inhibitor therefore typically includes structural elements that are known to be involved in the interactions, and may also have a restriction of its conformational flexibility, so as to avoid conformational changes that would affect or weaken its association with catalytic binding site.

The present inventors have uncovered that a series of structurally similar small molecules efficiently bind, covalently, to the Cys113 residue of Pin1, and have designed, based on these findings, and successfully practiced, small molecules that are capable of interacting with Pin1. The present inventors have identified that the structural features of the newly designed compounds that allow efficient interaction within the catalytic domain of Pin1, for example, such that reactivity with Cys113 is far higher than with other thiol groups.

In some embodiments, the compounds of the present invention, such as compounds of Formula I, IA, IB, II, III, or IV are selective inhibitors of Pin1. In some embodiments, the compounds of the present invention, such as compounds of Formula I, II, III, or IV are selective inhibitors of Pin1. In some embodiments, the compounds of the present invention, such as compounds of Formula I, IA, IB, II, III, IV, 4a-4g or pharmaceutically acceptable salt thereof are selective inhibitors of Pin1.

In some embodiments, the compound is such that, upon contacting the Pin1 catalytic binding site, one of its functional groups covalently binds the Cys113 residue of Pin1, and one or more other functional groups are in a proximity and orientation, as defined hereinabove, with respect to at least one another amino acid residue within the catalytic binding site of Pin1.

By “proximity and orientation” it is meant that, as discussed hereinabove, the functional group(s) are sufficiently close and properly oriented so as to strongly interact with the one or more amino acid residues (e.g., other than the Cys113) within the catalytic domain of the enzyme.

By “interacting” or “interact”, in the context of a functional group of the compound and an amino acid residue in the catalytic domain, it is meant a chemical interaction as a result of, for example, non-covalent interactions such as, but not limited to, hydrophobic interactions, including aromatic interactions, electrostatic interactions, Van der Waals interactions and hydrogen bonding. The interaction is such that results in the low dissociation constant of the compound-enzyme complex as disclosed herein.

The compounds described in some embodiments of any of the aspects of the present embodiments, and any combination thereof are characterized by electrophilic moiety and a rigid moiety that comprises at least one functional group that is capable of interacting with one or more amino acid residues in the catalytic domain of Pin1.

In some embodiments, the functional group(s) of the rigid moiety is/are capable of forming hydrogen bonds with hydrogen atoms of one or more amino acid residues in the catalytic domain of Pin1.

In some embodiments, the electrophilic moiety and the rigid moiety are arranged such that the electrophilic moiety is capable of covalently binding to the Cys113 residue of the Pin1 (SEQ ID NO: 1), and the rigid moiety is capable of forming hydrogen bonds with the Gln131 and His 157 residues of Pin1 (having the following amino acid sequence:

(SEQ ID NO: 1)
MADEEKLPPGWEKRMSRSSGRVYYFNHITNASQWERPSGNSSSGGKNGQ
GEPARVRCSHLLVKHSQSRRPSSWRQEKITRTKEEALELINGYIQKIKS
GEEDFESLASQFSDCSSAKARGDLGAFSRGQMQKPFEDASFALRTGEMS
GPVFTDSGIHIILRTE.

In some embodiments, the compound is such that when it contacts Pin1, the functional group(s) of the rigid moiety are in proximity and orientation with respect to the electrophilic group (prior to its covalent binding to Cys113), and to amino acid residues in the catalytic domain of Pin1 (e.g., the Gln131 and His 157 residues of Pin1), e.g., via hydrogen bonding, such that the electrophilic group is in proximity and orientation with respect to Cys113, thereby facilitating covalent binding of the Cys113 to the electrophilic group.

In some embodiments, the compound is such that when it contacts Pin1, the functional group(s) of the rigid moiety are in proximity and orientation with respect to the electrophilic group after its covalent binding to Cys113, that allow interaction, e.g., via hydrogen bonding, with other amino acid residues in the catalytic domain of Pin1 (e.g., with the Gln131 and His 157 residues of Pin1).

In some embodiments, the functional group (comprised by the rigid moiety) is capable of forming a hydrogen bond with a backbone amide hydrogen of the Gln131 and/or with an imidazole NH of the His157. In some embodiments, the rigid moiety comprises a functional group capable of forming a hydrogen bond with a backbone amide hydrogen of the Gln131, and another functional group capable of forming a hydrogen bond with an imidazole NH of the His157. In some embodiments, a distance between an atom of the functional group (e.g., O, S or N) and a nitrogen atom of Gln131 or His157 linked to the functional group via a hydrogen bond is in a range of from about 2.5 to 3.5 Å, optionally in a range of from about 2.7 to 3.3 Å.

Herein throughout, numbering of the amino acid residues of Pin1 is in accordance with SEQ ID NO: 1.

As used herein and known in the art, a “hydrogen bond” is a relatively weak bond that forms a type of dipole-dipole attraction which occurs when a hydrogen atom bonded to a strongly electronegative atom exists in the vicinity of another electronegative atom with a lone pair of electrons.

The hydrogen atom in a hydrogen bond is partly shared between two relatively electronegative atoms.

Hydrogen bonds typically have energies of about 1-3 kcal mol⁻¹ (4-13 kJ mol⁻¹), and their bond distances (measured from the hydrogen atom) typically range from about 1.5 to 2.6 Å.

A hydrogen-bond donor is the group that includes both the atom to which the hydrogen is more tightly linked and the hydrogen atom itself, whereas a hydrogen-bond acceptor is the atom less tightly linked to the hydrogen atom. The relatively electronegative atom to which the hydrogen atom is covalently bonded pulls electron density away from the hydrogen atom so that it develops a partial positive charge (δ⁺). Thus, it can interact with an atom having a partial negative charge (δ⁻) through an electrostatic interaction.

Atoms that typically participate in hydrogen bond interactions, both as donors and acceptors, include oxygen, nitrogen and fluorine. These atoms typically form a part of chemical group or moiety such as, for example, carbonyl, carboxylate, amide, hydroxyl, amine, imine, alkyl fluoride, F₂, and more. However, other electronegative atoms and chemical groups or moieties containing same may participate in hydrogen bonding.

In some of any of the embodiments described herein, the compound further comprising a hydrophobic moiety, e.g., attached to the electrophilic moiety and/or to the rigid moiety. In some embodiments, the hydrophobic moiety forms a hydrophobic interaction with Ser115, Leu122 and/or Met130 of Pin1.

Herein, the term “hydrophobic moiety” refers to a moiety for which a corresponding compound (i.e., a compound consisting of the moiety and one or more hydrogen atoms attached thereto) is water-insoluble, that is, a solubility of such a compound in water is less than 1 weight percent, e.g., at room temperature (at a pH of about 7). In some embodiments, R₁ of Formulas IA, IB, I, II, III or IV is hydrophobic moiety.

In some of any of the embodiments described herein, the functional moiety forming hydrogen bonds is an oxygen atom (O), a sulfur atom (S) and/or NH.

A plurality of functional moieties may optionally be the same or different and may optionally be attached to the same position in the rigid moiety (e.g., cyclic moiety) and/or at different positions.

In some of any of the embodiments described herein, two or more functional moieties forming hydrogen bonds are attached to the same atom, for example, a sulfur atom, in the rigid moiety. In some embodiments, the functional moieties are oxygen atoms, and two oxygen atoms attached to the sulfur atom form a sulfone (—S(═O)₂—) group. In some embodiments, the sulfur atom of the sulfone is a member of a ring, that is, a cyclic sulfone (e.g., a sulfolane or sulfolene).

In some embodiments, exemplified compounds of the compounds of Formula IA, IB, I, II, III and IV of this invention include compounds 4b-4g.

In some embodiments, the compounds of this invention the compounds of Formula IA, IB, I, II, III and IV or a pharmaceutically acceptable salt thereof or a pharmaceutically acceptable salt thereof or compounds 4b-4g or a pharmaceutically acceptable salt thereof show prolonged buffer stability than Sulfopin.

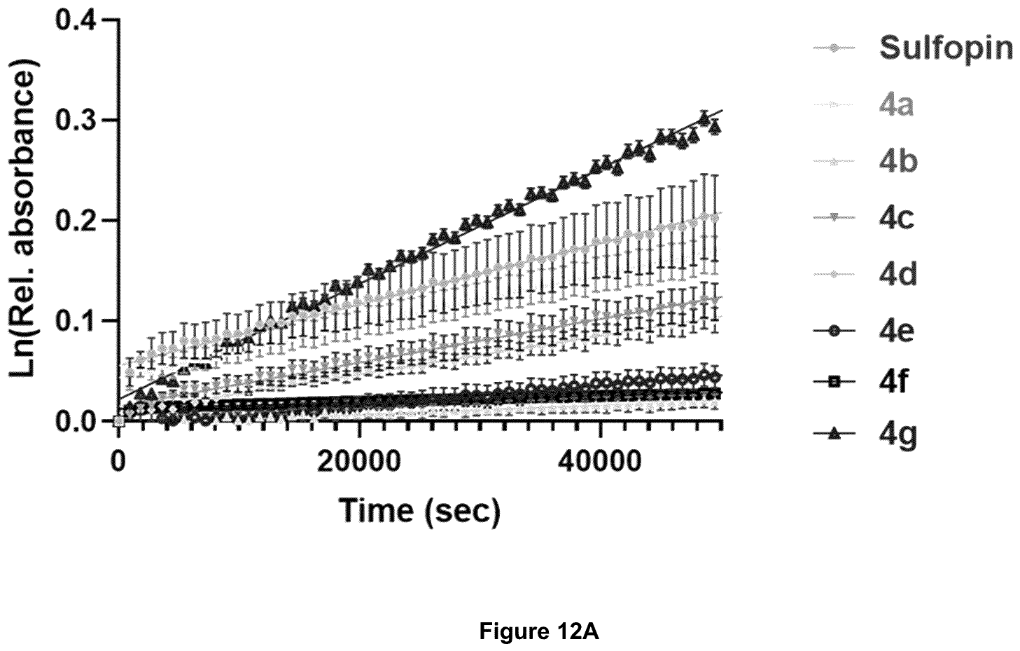

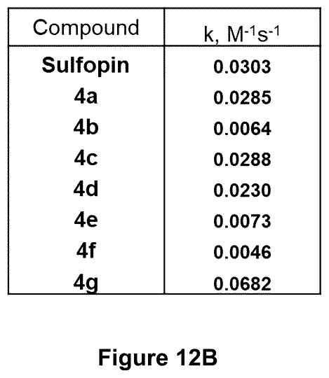

In some embodiments, the compounds of Formula IA, IB, I, II, III and IV or compounds 4b-4g or a pharmaceutically acceptable salt thereof show lower off-target thiol reactivity and higher selectivity for Pin1 relative to Sulfopin (FIG. 11C & FIGS. 12A, 12B)

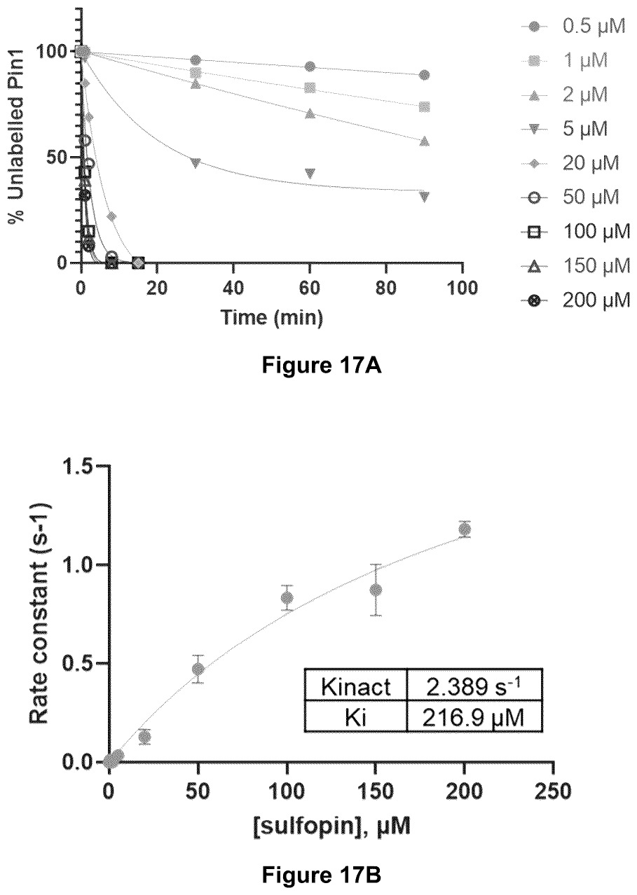

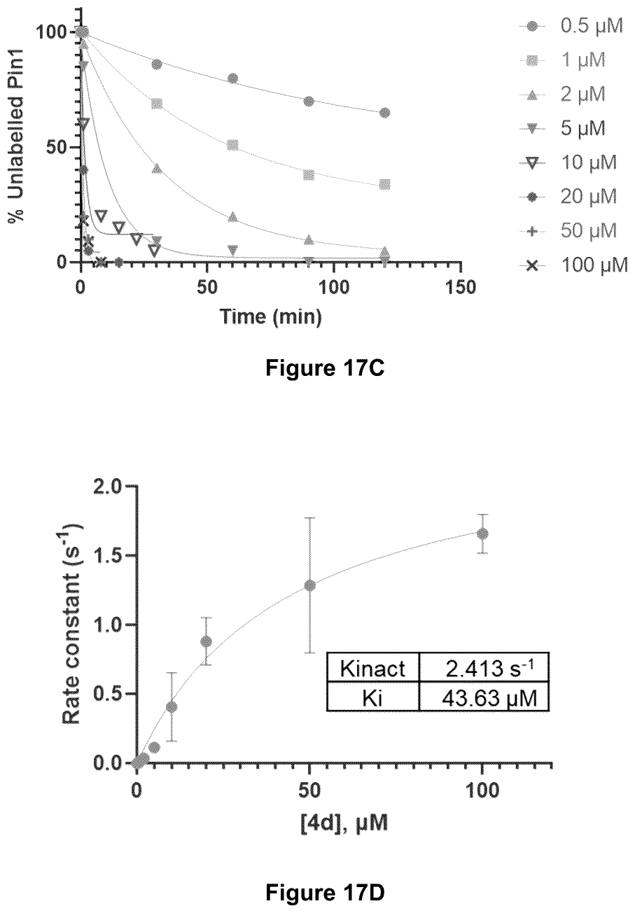

In some embodiments, and not to be bound to any theory, the compound of Formula IA, IB, I, II, III and IV or a pharmaceutically acceptable salt thereof or compounds 4b-4g or a pharmaceutically acceptable salt thereof bind to bind to a secondary pocket near the active site of Pin1. (FIG. 15), mediating additional interactions with Pin1 which is also supported by a five-times better K₁ value for 4d over Sulfopin (FIGS. 17A-17D).

In some embodiments, the Sulfamate compounds provided herein show similar proteomic selectivity and cellular engagement to Sulfopin.

According to some of any of the embodiments described herein, the compound of Formula IA, IB, I, II, III, IV or a pharmaceutically acceptable salt thereof is for use in modulating an activity of Pin1.

According to some of any of the embodiments described herein, the compound of Formula IA, IB, I, II, III, IV or a pharmaceutically acceptable salt thereof is for use in treating a condition in which modulating an activity of Pin1 is beneficial.

According to some of any of the embodiments described herein relating to a condition in which modulating an activity of Pin1 is beneficial, the condition is a proliferative disease or disorder and/or an immune disease or disorder.

According to some of any of the embodiments described herein relating to a proliferative disease or disorder, the proliferative disease or disorder is a cancer.

According to some of any of the embodiments described herein relating to a proliferative disease or disorder, the proliferative disease or disorder is selected from the group consisting of a pancreatic cancer, a neuroblastoma, a prostate cancer, an ovarian carcinoma, and a breast adenocarcinoma.

According to some of any of the embodiments described herein relating to a proliferative disease or disorder, the proliferative disease or disorder is a pancreatic cancer.

According to some of any of the embodiments described herein relating to a proliferative disease or disorder, the proliferative disease or disorder is a neuroblastoma.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Herein, the terms “electrophile” and “electrophilic moiety” refer to any moiety capable of reacting with a nucleophile (e.g., a moiety having a lone pair of electrons, a negative charge, a partial negative charge and/or an excess of electrons, for example a thiol group.

A “leaving group” as used herein and in the art describes a labile atom, group or chemical moiety that readily undergoes detachment from an organic molecule during a chemical reaction, while the detachment is typically facilitated by the relative stability of the leaving atom, group or moiety thereupon. In other embodiments, the leaving group is sulfamic acid.

In some embodiments, a compound exhibiting low reactivity with a thiol is a compound for which the rate constant k is no more than about 3×10⁻⁷ M/sec. In some embodiments, the rate constant k is no more than about 2×10⁻⁷ M/sec. In some embodiments, the rate constant k is no more than about 10⁻⁷ M/sec. In some embodiments, the rate constant k is no more than about 5×10⁻⁸ M/sec. In some embodiments, the rate constant k is no more than about 3×10⁻⁸ M/sec. In some embodiments, the rate constant k is no more than about 2×10⁻⁸ M/sec. In some embodiments, the rate constant k is no more than about 10⁻⁸ M/sec. In some embodiments, the rate constant k is no more than about 5×10⁻⁹ M/sec.

The present invention, in some embodiments thereof, relates to pharmacology, and more particularly, but not exclusively, to compounds of Formula IA, IB, I, II, III, IV or a pharmaceutically acceptable salt thereof that covalently bind to, and/or modulate the activity of, Pin1 and to uses thereof in, for example, treating diseases associated with Pin1 activity.

The present inventors have uncovered new compounds for effectively and selectively modulating the activity of Pin1, by laboriously screening compounds capable of covalently reacting with the protein and studying the relationship between structure and activity and off-target toxicity. While reducing the present invention to practice, the inventors have uncovered exemplary compounds which selectively and covalently react with the active site (catalytic domain) of Pin1, as well as the effects of selective modulation of Pin1 activity in various physiological models.

As used herein, the phrase “catalytic domain” describes a region of an enzyme, Pin1, in which the catalytic reaction occurs. This phrase therefore describes this part of an enzyme in which the substrate and/or other components that participate in the catalytic reaction interacts with the enzyme. In the context of the present embodiments, this phrase is particularly used to describe this part of an enzyme (a Pin1) to which the substrate binds during the catalytic activity (e.g., phosphorylation). This phrase is therefore also referred to herein and in the art, interchangeably, as “substrate binding pocket”, “catalytic site” “active site” and the like.

As used herein, the phrases “binding site”, “catalytic binding site” or “binding subsite”, which are used herein interchangeably, describe a specific site in the catalytic domain that includes one or more reactive groups through which the interactions of the enzyme with the substrate and/or an inhibitor can be affected. Typically, the binding site is composed of one or two amino acid residues, whereby the interactions typically involve reactive groups at the side chains of these amino acids.

As is well known in the art, when an enzyme interacts with a substrate or an inhibitor, the initial interaction rapidly induces conformational changes, in the enzyme and/or substrate and/or inhibitor, that strengthen binding and bring enzyme's binding sites close to functional groups in the substrate or inhibitor. Enzyme-substrate/inhibitor interactions orient reactive groups present in both the enzyme and the substrate/inhibitor and bring them into proximity with one another. The binding of the substrate/inhibitor to the enzyme aligns the reactive groups so that the relevant molecular orbitals overlap.

Thus, an inhibitor of an enzyme is typically associated with the catalytic domain of the enzyme such that the reactive groups of the inhibitor are positioned in sufficient proximity to corresponding reactive groups (typically side chains of amino acid residues) in the enzyme catalytic binding site, so as to allow the presence of an effective concentration of the inhibitor in the catalytic binding site and, in addition, the reactive groups of the inhibitor are positioned in a proper orientation, to allow overlap and thus a strong chemical interaction and low dissociation. An inhibitor therefore typically includes structural elements that are known to be involved in the interactions, and may also have a restriction of its conformational flexibility, so as to avoid conformational changes that would affect or weaken its association with catalytic binding site.

Embodiments of the present invention therefore generally relate to newly designed small molecules and to uses thereof, e.g., in modulating an activity of Pin1. The present inventors have identified that the Sulfamate compounds that allow efficient interaction within the catalytic domain of Pin1, for example, such that reactivity with Cys113 is far higher than with other thiol groups.

According to some embodiments of the present invention, the Sulfamate compound provided herein provides strong association with the catalytic binding site of Pin1.

In some embodiments, the compound is such that, upon contacting the Pin1 catalytic binding site, one of its functional groups covalently binds the Cys113 residue of Pin1, and one or more other functional groups are in a proximity and orientation, as defined hereinabove, with respect to at least one another amino acid residue within the catalytic binding site of Pin1.

According to an aspect of some embodiments of the invention, there is provided a use of one or more compounds according to any of the embodiments described herein in the manufacture of a medicament for treating a condition in which modulating an activity of Pin1 is beneficial.

According to an aspect of some embodiments of the invention, there is provided a method of treating a condition in which modulating an activity of Pin1 is beneficial, the method comprising administering to a subject in need thereof one or more Sulfamate compound according to any of the embodiments described herein.

According to an aspect of some embodiments of the invention, there is provided a method of modulating an activity of Pin1, the method comprising contacting the Pin1 with one or more Sulfamate compound according to any of the embodiments described herein. Modulation of Pin1 activity may optionally be effected in vitro (e.g., for research purposes) or in vivo (e.g., wherein contacting is effected by administration to a subject in need thereof).

Herein, the term “modulation” encompasses up-regulation as well as down-regulation (e.g., by antagonistic binding) of an activity (e.g., of Pin1), and may be effected, e.g., by interacting with an active site (e.g., of Pin1) or by modulating degradation of the protein.

In some embodiments, provided herein is a method of treating a disease or condition associated with Pin1 activity, comprising administering a compound of this invention, or a pharmaceutically acceptable salt thereof, or a pharmaceutical composition of this invention. In another embodiment, the compound of this invention is I, II, III, or IV. In another embodiment, the compound of this inventions is I, IA, IB, II, III, or IV.

In some embodiments, provided herein is a method of treating a disease or condition associated with Pin1 activity, comprising administering a compound of Formula I, II, III, or IV or a pharmaceutically acceptable salt thereof, or a pharmaceutical composition comprising a compound of Formula I, II, III, or IV or a pharmaceutically acceptable salt thereof.

In some embodiments, provided herein is a method of treating a disease or condition associated with Pin1 activity, comprising administering a compound of this invention, or a pharmaceutically acceptable salt thereof, or a pharmaceutical composition of this invention, wherein the disease or condition is a proliferative disease or disorder.

In some embodiments, provided herein is a method of treating a disease or condition associated with Pin1 activity, comprising administering a compound of this invention, or a pharmaceutically acceptable salt thereof, or a pharmaceutical composition of this invention, wherein the disease is a cancer.

In some embodiments, provided herein is a method of treating a disease or condition associated with Pin1 activity, comprising administering a compound of this invention, or a pharmaceutically acceptable salt thereof, or a pharmaceutical composition of this invention, wherein the disease is selected from the group consisting of a pancreatic cancer, a neuroblastoma, a prostate cancer, an ovarian carcinoma, and a breast adenocarcinoma.

In some embodiment, provided herein is a method of treating a disease or condition associated with Pin1 activity, comprising administering a compound of this invention, or a pharmaceutically acceptable salt thereof, or a pharmaceutical composition of this invention, wherein the disease or condition is an immune disease or disorder. In some of any of the respective embodiments described herein, according to any of the aspects described herein, modulating an activity of Pin1 comprises inhibiting an activity of Pin1.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease but has not yet been diagnosed as having the disease.

As used herein, the term “subject” includes mammals, optionally human beings at any age which suffer from the pathology. Optionally, this term encompasses individuals who are at risk to develop the pathology.

Examples of conditions in which modulating an activity of Pin1 may be beneficial include, without limitation, proliferative diseases or disorders and immune diseases or disorders. The proliferative disease or disorder may be, for example, a cancer or pre-cancer.

In some of any of the respective embodiments described herein, treatment is for inhibiting initiation of a tumor (optionally neuroblastoma), for example, inhibiting metastases.

Non-limiting examples of Pin1-associated cancers which can be treated according to some of the respective embodiments of the invention can be any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

Pancreatic cancer (e.g., pancreatic adenocarcinoma) is an exemplary type of cancer treatable according to some embodiments of the invention.

Pre-cancers are well characterized and known in the art (refer, for example, to Berman J J. and Henson D E., 2003. Classifying the precancers: a metadata approach. BMC Med Inform Decis Mak. 3:8, which is incorporated herein by reference in its entirety). Classes of pre-cancers amenable to treatment via the method of the invention include acquired small or microscopic pre-cancers, acquired large lesions with nuclear atypia, precursor lesions occurring with inherited hyperplastic syndromes that progress to cancer, and acquired diffuse hyperplasias and diffuse metaplasias. Examples of small or microscopic pre-cancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Examples of precursor lesions occurring with inherited hyperplastic syndromes that progress to cancer include atypical mole syndrome, C cell adenomatosis and MEA. Examples of acquired diffuse hyperplasias and diffuse metaplasias include AIDS, atypical lymphoid hyperplasia, Paget's disease of bone, post-transplant lymphoproliferative disease and ulcerative colitis.

Therapeutic regimens for treatment of cancer suitable for combination with one or more sulfonate or sulfamate compounds according to any of the respective embodiments of the invention include, but are not limited to chemotherapy, radiotherapy, phototherapy and photodynamic therapy, surgery, nutritional therapy, ablative therapy, combined radiotherapy and chemotherapy, brachiotherapy, proton beam therapy, immunotherapy, cellular therapy and photon beam radiosurgical therapy.

Alternative or additional chemotherapeutic drugs (e.g., anti-cancer drugs) that may optionally be co-administered with the Sulfamate compounds of the invention include, but are not limited to acivicin, aclarubicin, acodazole, acronine, adozelesin, aldesleukin, altretamine, ambomycin, ametantrone, aminoglutethimide, amsacrine, anastrozole, anthramycin, asparaginase, asperlin, azacitidine, azetepa, azotomycin, batimastat, benzodepa, bicalutamide, bisantrene, bisnafide, bizelesin, bleomycin, brequinar, bropirimine, busulfan, cactinomycin, calusterone, caracemide, carbetimer, carboplatin, carmustine, carubicin, carzelesin, cedefingol, chlorambucil, cirolemycin, cisplatin, cladribine, crisnatol, cyclophosphamide, cytarabine, dacarbazine, dactinomycin, daunorubicin, decitabine, dexormaplatin, dezaguanine, diaziquone, docetaxel, doxorubicin, droloxifene, dromostanolone, duazomycin, edatrexate, eflornithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, gemcitabine, hydroxyurea, idarubicin, ifosfamide, ilmofosine, interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine, megestrol, melengestrol, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nocodazole, nogalamycin, ormaplatin, oxisuran, paclitaxel, pegaspargase, peliomycin, pentamustine, peplomycin, perfosfamide, pipobroman, piposulfan, piroxantrone, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine, vindesine, vinepidine, vinglycinate, vinleurosine, vinorelbine, vinrosidine, vinzolidine, vorozole, zeniplatin, zinostatin, zorubicin, and any pharmaceutically acceptable salts thereof. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division), which are hereby incorporated by reference in their entirety.

It is expected that during the life of a patent maturing from this application many relevant drugs will be developed and the scope of the terms “anti-cancer agent”, “chemotherapeutic drug”, “antineoplastic agent” and the like are intended to include all such new technologies apriori.

In some embodiments, an additional anti-cancer agents may optionally be selected in accordance with the condition to be treated, for example, by selecting an agent for use in treating a condition for which the agent (per se) has already been approved, e.g., as indicated in Table 2

TABLE 2
Anti-cancer agents

| | | |
|---|---|---|
| Aldesleukin | Proleukin | |
| alitretinoin | Panretin | Topical treatment of cutaneous lesions in |
| | | patients with AIDS-related Kaposi's sarcoma. |
| allopurinol | Zyloprim | Patients with leukemia, lymphoma and solid |
| | | tumor malignancies who are receiving cancer |
| | | therapy which causes elevations of serum and |
| | | urinary uric acid levels and who cannot |
| | | tolerate oral therapy. |
| altretamine | Hexalen | Single agent palliative treatment of patients |
| | | with persistent or recurrent ovarian cancer |
| | | following first-line therapy with a cisplatin |
| | | and/or alkylating agent based combination. |
| amifostine | Ethyol | To reduce the cumulative renal toxicity |
| | | associated with repeated administration of |
| | | cisplatin in patients with advanced ovarian |
| | | cancer |
| amifostine | Ethyol | To reduce post-radiation xerostomia for head |
| | | and neck cancer where the radiation port |
| | | includes a substantial portion of the parotid |
| | | glands. |
| anastrozole | Arimidex | Treatment of advanced breast cancer in |
| | | postmenopausal women with disease |
| | | progression following tamoxifen therapy. |
| anastrozole | Arimidex | For first-line treatment of postmenopausal |
| | | women with hormone receptor positive or |
| | | hormone receptor unknown locally advanced |
| | | or metastatic breast cancer. |
| arsenic trioxide | Trisenox | Second line treatment of relapsed or |
| | | refractory APL following ATRA plus an |
| | | anthracycline. |
| Asparaginase | Elspar | ELSPAR is indicated in the therapy of |
| | | patients with acute lymphocytic leukemia. |
| | | This agent is useful primarily in combination |
| | | with other chemotherapeutic agents in the |
| | | induction of remissions of the disease in |
| | | pediatric patients. |
| BCG Live | TICE BCG | |
| bexarotene capsules | Targretin | For the treatment by oral capsule of |
| | | cutaneous manifestations of cutaneous T-cell |
| | | lymphoma in patients who are refractory to at |
| | | least one prior systemic therapy. |
| bexarotene gel | Targretin | For the topical treatment of cutaneous |
| | | manifestations of cutaneous T-cell |
| | | lymphoma in patients who are refractory to at |
| | | least one prior systemic therapy. |
| bleomycin | Blenoxane | Sclerosing agent for the treatment of |
| | | malignant pleural effusion (MPE) and |
| | | prevention of recurrent pleural effusions. |
| busulfan intravenous | Busulfex | Use in combination with cyclophoshamide as |
| | | conditioning regimen prior to allogeneic |
| | | hematopoietic progenitor cell transplantation |
| | | for chronic myelogenous leukemia. |
| busulfan oral | Myleran | Chronic Myelogenous Leukemia- palliative |
| | | therapy |
| calusterone | Methosarb | |
| capecitabine | Xeloda | Initial therapy of patients with metastatic |
| | | colorectal carcinoma when treatment with |
| | | fluoropyrimidine therapy alone is preferred. |
| | | Combination chemotherapy has shown a |
| | | survival benefit compared to 5-FU/LV alone. |
| | | A survival benefit over 5_FU/LV has not |
| | | been demonstrated with Xeloda |
| | | monotherapy. |
| capecitabine | Xeloda | Treatment in combination with docetaxel of |
| | | patients with metastatic breast cancer after |
| | | failure of prior anthracycline containing |
| | | chemotherapy |
| carboplatin | Paraplatin | Palliative treatment of patients with ovarian |
| | | carcinoma recurrent after prior |
| | | chemotherapy, including patients who have |
| | | been previously treated with cisplatin. |
| carboplatin | Paraplatin | Initial chemotherapy of advanced ovarian |
| | | carcinoma in combination with other |
| | | approved chemotherapeutic agents. |
| carmustine | BCNU, BiCNU | |
| carmustine with | Gliadel Wafer | For use in addition to surgery to prolong |
| Polifeprosan 20 Implant | | survival in patients with recurrent |
| | | glioblastoma multiforme who qualify for |
| | | surgery. |
| chlorambucil | Leukeran | Chronic Lymphocytic Leukemia- palliative |
| | | therapy |
| cisplatin | Platinol | Metastatic testicular-in established |
| | | combination therapy with other approved |
| | | chemotherapeutic agents in patients with |
| | | metastatic testicular tumors whoc have |
| | | already received appropriate surgical and/or |
| | | radiotherapeutic procedures. An established |
| | | combination therapy consists of Platinol, |
| | | Blenoxane and Velbam. |
| cisplatin | Platinol | Metastatic ovarian tumors - in established |
| | | combination therapy with other approved |
| | | chemotherapeutic agents: Ovarian-in |
| | | established combination therapy with other |
| | | approved chemotherapeutic agents in patients |
| | | with metastatic ovarian tumors who have |
| | | already received appropriate surgical and/or |
| | | radiotherapeutic procedures. An established |
| | | combination consists of Platinol and |
| | | Adriamycin. Platinol, as a single agent, is |
| | | indicated as secondary therapy in patients |
| | | with metastatic ovarian tumors refractory to |
| | | standard chemotherapy who have not |
| | | previously received Platinol therapy. |
| cisplatin | Platinol | as a single agent for patients with transitional |
| | | cell bladder cancer which is no longer |
| | | amenable to local treatments such as surgery |
| | | and/or radiotherapy. |
| cladribine | Leustatin, 2-CdA | Treatment of active hairy cell leukemia. |
| cyclophosphamide | Cytoxan, Neosar | |
| cyclophosphamide | Cytoxan Injection | |
| cyclophosphamide | Cytoxan Tablet | |
| cytarabine | Cytosar-U | |
| dacarbazine | DTIC-Dome | |
| dactinomycin, | Cosmegan | |
| actinomycin D | | |
| Darbepoetin alfa | Aranesp | Treatment of anemia associated with chronic |
| | | renal failure. |
| Darbepoetin alfa | Aranesp | Aranesp is indicated for the treatment of |
| | | anemia in patients with non- myeloid |
| | | malignancies where anemia is due to the |
| | | effect of concomitantly administered |
| | | chemotherapy. |
| daunorubicin liposomal | DanuoXome | First line cytotoxic therapy for advanced, |
| | | HIV related Kaposi's sarcoma. |
| daunorubicin, | Daunorubicin | Leukemia/myelogenous/monocytic/erythroid |
| daunomycin | | of adults/remission induction in acute |
| | | lymphocytic leukemia of children and adults. |
| daunorubicin, | Cerubidine | In combination with approved anticancer |
| daunomycin | | drugs for induction of remission in adult |
| | | ALL. |
| dexrazoxane | Zinecard | Accel. Approv. (clinical benefit subsequently |
| | | established) Prevention of cardiomyopathy |
| | | associated with doxorubicin administration |
| dexrazoxane | Zinecard | reducing the incidence and severity of |
| | | cardiomyopathy associated with doxorubicin |
| | | administration in women with metastatic |
| | | breast cancer who have received a cumulative |
| | | doxorubicin dose of 300 mg/m2 and who will |
| | | continue to receive doxorubicin therapy to |
| | | maintain tumor control. It is not |
| | | recommended for use with the initiation of |
| | | doxorubicin therapy. |
| docetaxel | Taxotere | Accel. Approv. (clinical benefit subsequently |
| | | established) Treatment of patients with |
| | | locally advanced or metastatic breast cancer |
| | | who have progressed during anthracycline- |
| | | based therapy or have relapsed during |
| | | anthracycline-based adjuvant therapy. |
| docetaxel | Taxotere | For the treatment of locally advanced or |
| | | metastatic breast cancer which has |
| | | progressed during anthracycline-based |
| | | treatment or relapsed during anthracycline- |
| | | based adjuvant therapy. |
| docetaxel | Taxotere | For locally advanced or metastatic non-small |
| | | cell lung cancer after failure of prior |
| | | platinum-based chemotherapy. |
| docetaxel | Taxotere | in combination with cisplatin for the |
| | | treatment of patients with unresectable, |
| | | locally advanced or metastatic non-small cell |
| | | lung cancer who have not previously received |
| | | chemotherapy for this condition. |
| doxorubicin | Adriamycin, Rubex | |
| doxorubicin | Adriamycin PFS | Antibiotic, antitumor agent. |
| | Injectionintravenous | |
| | injection | |
| DROMOSTANOLONE | DROMOSTANOLONE | |
| PROPIONATE | | |
| DROMOSTANOLONE | MASTERONE | |
| PROPIONATE | INJECTION | |
| Elliott's B Solution | Elliott's B Solution | Diluent for the intrathecal administration of |
| | | methotrexate sodium and cytarabine for the |
| | | prevention or treatment of meningeal |
| | | leukemia or lymphocytic lymphoma. |
| epirubicin | Ellence | A component of adjuvant therapy in patients |
| | | with evidence of axillary node tumor |
| | | involvement following resection of primary |
| | | breast cancer. |
| Epoetin alfa | epogen | EPOGENB is indicated for the reatment of |
| | | anemia related to therapy with zidovudine in |
| | | HIV- infected patients. EPOGENB is |
| | | indicated to elevate or maintain the red blood |
| | | cell level (as manifested by the hematocrit or |
| | | hemoglobin determinations) and to decrease |
| | | the need for transfusions in these patients. |
| | | EPOGEND is not indicated for the treatment |
| | | of anemia in HIV-infected patients due to |
| | | other factors such as iron or folate |
| | | deficiencies, hemolysis or gastrointestinal |
| | | bleeding, which should be managed |
| | | appropriately. |
| Epoetin alfa | epogen | EPOGENB is indicated for the treatment of |
| | | anemic patients (hemoglobin >10 to _<13 |
| | | g/dL) scheduled to undergo elective, |
| | | noncardiac, nonvascular surgery to reduce |
| | | the need for allogeneic blood transfusions. |
| Epoetin alfa | epogen | EPOGENB is indicated for the treatment of |
| | | anemia in patients with non-myeloid |
| | | malignancies where anemia is due to the |
| | | effect of concomitantly administered |
| | | chemotherapy. EPOGEND is indicated to |
| | | decrease the need for transfusions in patients |
| | | who will be receiving concomitant |
| | | chemotherapy for a minimum of 2 months. |
| | | EPOGENB is not indicated for the treatment |
| | | of anemia in cancer patients due to other |
| | | factors such as iron or folate deficiencies, |
| | | hemolysis or gastrointestinal bleeding, which |
| | | should be managed appropriately. |
| Epoetin alfa | epogen | EPOGEN is indicated for the treatment of |
| | | anemia associated with CRF, including |
| | | patients on dialysis (ESRD) and patients not |
| | | on dialysis. |
| estramustine | Emcyt | palliation of prostate cancer |
| etoposide phosphate | Etopophos | Management of refractory testicular tumors, |
| | | in combination with other approved |
| | | chemotherapeutic agents. |
| etoposide phosphate | Etopophos | Management of small cell lung cancer, first- |
| | | line, in combination with other approved |
| | | chemotherapeutic agents. |
| etoposide phosphate | Etopophos | Management of refractory testicular tumors |
| | | and small cell lung cancer. |
| etoposide, VP-16 | Vepesid | Refractory testicular tumors-in combination |
| | | therapy with other approved |
| | | chemotherapeutic agents in patients with |
| | | refractory testicular tumors who have already |
| | | received appropriate surgical, |
| | | chemotherapeutic and radiotherapeutic |
| | | therapy. |
| etoposide, VP-16 | VePesid | In combination with other approved |
| | | chemotherapeutic agents as first line |
| | | treatment in patients with small cell lung |
| | | cancer. |
| etoposide, VP-16 | Vepesid | In combination with other approved |
| | | chemotherapeutic agents as first line |
| | | treatment in patients with small cell lung |
| | | cancer. |
| exemestane | Aromasin | Treatment of advance breast cancer in |
| | | postmenopausal women whose disease has |
| | | progressed following tamoxifen therapy. |
| Filgrastim | Neupogen | NEUPOGEN is indicated to reduce the |
| | | duration of neutropenia and neutropenia- |
| | | related clinical sequelae, eg, febrile |
| | | neutropenia, in patients with nonmyeloid |
| | | malignancies undergoing myeloablative |
| | | chemotherapy followed by marrow |
| | | transplantation. |
| Filgrastim | Neupogen | NEUPOGEN is indicated to decrease the |
| | | incidence of infection, as manifested by |
| | | febrile neutropenia, in patients with |
| | | nonmyeloid malignancies receiving |
| | | myelosuppressive anticancer drugs |
| | | associated with a significant incidence of |
| | | severe neutropenia with fever. |
| Filgrastim | Neupogen | NEUPOGEN is indicated for reducing the |
| | | time to neutrophil recovery and the duration |
| | | of fever, following induction or consolidation |
| | | hemotherapy treatment of adults with AML. |
| floxuridine | FUDR | |
| (intraarterial) | | |
| fludarabine | Fludara | Palliative treatment of patients with B-cell |
| | | lymphocytic leukemia (CLL) who have not |
| | | responded or have progressed during |
| | | treatment with at least one standard |
| | | alkylating agent containing regimen. |
| fluorouracil, 5-FU | Adrucil | prolong survival in combination with |
| | | leucovorin |
| fulvestrant | Faslodex | the treatment of hormone receptor-positive |
| | | metastatic breast cancer in postmenopausal |
| | | women with disease progression following |
| | | antiestrogen therapy |
| gemcitabine | Gemzar | Treatment of patients with locally advanced |
| | | (nonresectable stage II or III) or metastatic |
| | | (stage IV) adenocarcinoma of the pancreas. |
| | | Indicated for first-line treatment and for |
| | | patients previously treated with a 5- |
| | | fluorouracil-containing regimen. |
| gemcitabine | Gemzar | For use in combination with cisplatin for the |
| | | first-line treatment of patients with |
| | | inoperable, locally advanced (Stage IIIA or |
| | | IIIB) or metastatic (Stage IV) non-small cell |
| | | lung cancer. |
| goserelin acetate | Zoladex Implant | Palliative treatment of advanced breast |
| | | cancer in pre- and perimenopausal women. |
| goserelin acetate | Zoladex | |
| hydroxyurea | Hydrea | Decrease need for transfusions in sickle cell |
| | | anemia |
| idarubicin | Idamycin | For use in combination with other approved |
| | | antileukemic drugs for the treatment of acute |
| | | myeloid leukemia (AML) in adults. |
| idarubicin | Idamycin | In combination with other approved |
| | | antileukemic drugs for the treatment of acute |
| | | non-lymphocytic leukemia in adults. |
| ifosfamide | IFEX | Third line chemotherapy of germ cell |
| | | testicular cancer when used in combination |
| | | with certain other approved antineoplastic |
| | | agents. |
| Interferon alfa-2a | Roferon-A | |
| Interferon alfa-2b | Intron A | Interferon alfa-2b, recombinant for injection |
| | | is indicated as adjuvant to surgical treatment |
| | | in patients 18 years of age or older with |
| | | malignant melanoma who are free of disease |
| | | but at high risk for systemic recurrence within |
| | | 56 days of surgery. |
| Interferon alfa-2b | Intron A | Interferon alfa-2b, recombinant for Injection |
| | | is indicated for the initial treatment of |
| | | clinically aggressive follicular Non- |
| | | Hodgkin's Lymphoma in conjunction with |
| | | anthracycline-containing combination |
| | | chemotherapy in patients 18 years of age or |
| | | older. |
| Interferon alfa-2b | Intron A | Interferon alfa-2b, recombinant for Injection |
| | | is indicated for intralesional treatment of |
| | | selected patients 18 years of age or older with |
| | | condylomata acuminata involving external |
| | | surfaces of the genital and perianal areas. |
| Interferon alfa-2b | Intron A | Interferon alfa-2b, recombinant for Injection |
| | | is indicated for the treatment of chronic |
| | | hepatitis C in patients 18 years of age or older |
| | | with compensated liver disease who have a |
| | | history of blood or blood-product exposure |
| | | and/or are HCV antibody positive. |
| Interferon alfa-2b | Intron A | Interferon alfa-2b, recombinant for Injection |
| | | is indicated for the treatment of chronic |
| | | hepatitis B in patients 18 years of age or older |
| | | with compensated liver disease and HBV |
| | | replication. |
| Interferon alfa-2b | Intron A | Interferon alfa-2b, recombinant for Injection |
| | | is indicated for the treatment of patients 18 |
| | | years of age or older with hairy cell leukemia. |
| Interferon alfa-2b | Intron A | Interferon alfa-2b, recombinant for Injection |
| | | is indicated for the treatment of selected |
| | | patients 18 years of age or older with AIDS- |
| | | Related Kaposi's Sarcoma. The likelihood of |
| | | response to INTRON A therapy is greater in |
| | | patients who are without systemic symptoms, |
| | | who have limited lymphadenopathy and who |
| | | have a relatively intact immune system as |
| | | indicated by total CD4 count. |
| irinotecan | Camptosar | Accel. Approv. (clinical benefit subsequently |
| | | established) Treatment of patients with |
| | | metastatic carcinoma of the colon or rectum |
| | | whose disease has recurred or progressed |
| | | following 5-FU-based therapy. |
| irinotecan | Camptosar | Follow up of treatment of metastatic |
| | | carcinoma of the colon or rectum whose |
| | | disease has recurred or progressed following |
| | | 5-FU-based therapy. |
| irinotecan | Camptosar | For first line treatment in combination with 5- |
| | | FU/leucovorin of metastatic carcinoma of the |
| | | colon or rectum. |
| letrozole | Femara | Treatment of advanced breast cancer in |
| | | postmenopausal women. |
| letrozole | Femara | First-line treatment of postmenopausal |
| | | women with hormone receptor positive or |
| | | hormone receptor unknown locally advanced |
| | | or metastatic breast cancer. |
| letrozole | Femara | |
| leucovorin | Wellcovorin, | Leucovorin calcium is indicated for use in |
| | Leucovorin | combination with 5-fluorouracil to prolong |
| | | survival in the palliative treatment of patients |
| | | with advanced colorectal cancer. |
| leucovorin | Leucovorin | In combination with fluorouracil to prolong |
| | | survival in the palliative treatment of patients |
| | | with advanced colorectal cancer. |
| levamisole | Ergamisol | Adjuvant treatment in combination with 5- |
| | | fluorouracil after surgical resection in |
| | | patients with Dukes' Stage C colon cancer. |
| lomustine, CCNU | CeeBU | |
| meclorethamine, | Mustargen | |
| nitrogen mustard | | |
| megestrol acetate | Megace | |
| melphalan, L-PAM | Alkeran | Systemic administration for palliative |
| | | treatment of patients with multiple myeloma |
| | | for whom oral therapy is not appropriate. |
| mercaptopurine, 6-MP | Purinethol | |
| mesna | Mesnex | Prevention of ifosfamide-induced |
| | | hemorrhagic cystitis |
| methotrexate | Methotrexate | osteosarcoma |
| methoxsalen | Uvadex | For the use of UVADEX with the UVAR |
| | | Photopheresis System in the palliative |
| | | treatment of the skin manifestations of |
| | | cutaneous T-cell lymphoma (CTCL) that is |
| | | unresponsive to other forms of treatment. |
| mitomycin C | Mutamycin | |
| mitomycin C | Mitozytrex | therapy of disseminated adenocarcinoma of |
| | | the stomach or pancreas in proven |
| | | combinations with other approved |
| | | chemotherapeutic agents and as palliative |
| | | treatment when other modalities have failed. |
| mitotane | Lysodren | |
| mitoxantrone | Novantrone | For use in combination with corticosteroids |
| | | as initial chemotherapy for the treatment of |
| | | patients with pain related to advanced |
| | | hormone-refractory prostate cancer. |
| mitoxantrone | Novantrone | For use with other approved drugs in the |
| | | initial therapy for acute nonlymphocytic |
| | | leukemia (ANLL) in adults. |
| nandrolone | Durabolin-50 | |
| phenpropionate | | |
| Nofetumomab | Verluma | |
| Oprelvekin | Neumega | Neumega is indicated for the prevention of |
| | | severe thrombocytopenia and the reduction of |
| | | the need for platelet transfusions following |
| | | myelosuppressive chemotherapy in adult |
| | | patients with nonmyeloid malignancies who |
| | | are at high risk of severe thrombocytopenia. |
| paclitaxel | Paxene | treatment of advanced AIDS-related Kaposi's |
| | | sarcoma after failure of first line or |
| | | subsequent systemic chemotherapy |
| paclitaxel | Taxol | Treatment of patients with metastatic |
| | | carcinoma of the ovary after failure of first- |
| | | line or subsequent chemotherapy. |
| paclitaxel | Taxol | Treatment of breast cancer after failure of |
| | | combination chemotherapy for metastatic |
| | | disease or relapse within 6 months of |
| | | adjuvant chemotherapy. Prior therapy should |
| | | have included an anthracycline unless |
| | | clinically contraindicated. |
| paclitaxel | Taxol | New dosing regimen for patients who have |
| | | failed initial or subsequent chemotherapy for |
| | | metastatic carcinoma of the ovary |
| paclitaxel | Taxol | second line therapy for AIDS related Kaposi's |
| | | sarcoma. |
| paclitaxel | Taxol | For first-line therapy for the treatment of |
| | | advanced carcinoma of the ovary in |
| | | combination with cisplatin. |
| paclitaxel | Taxol | for use in combination with cisplatin, for the |
| | | first-line treatment of non-small cell lung |
| | | cancer in patients who are not candidates for |
| | | potentially curative surgery and/or radiation |
| | | therapy. |
| paclitaxel | Taxol | For the adjuvant treatment of node-positive |
| | | breast cancer administered sequentially to |
| | | standard doxorubicin-containing |
| | | combination therapy. |
| paclitaxel | Taxol | First line ovarian cancer with 3 hour infusion. |
| pamidronate | Aredia | Treatment of osteolytic bone metastases of |
| | | breast cancer in conjunction with standard |
| | | antineoplastic therapy. |
| pegademase | Adagen (Pegademase | Enzyme replacement therapy for patients |
| | Bovine) | with severe combined immunodeficiency asa |
| | | result of adenosine deaminase deficiency. |
| Pegaspargase | Oncaspar | |
| Pegfilgrastim | Neulasta | Neulasta is indicated to decrease the |
| | | incidence of infection, as manifested by |
| | | febrile neutropenia, in patients with non- |
| | | myeloid malignancies receiving |
| | | myelosuppressive anti-cancer drugs |
| | | associated with a clinically significant |
| | | incidence of febrile neutropenia. |
| pentostatin | Nipent | Single agent treatment for adult patients with |
| | | alpha interferon refractory hairy cell |
| | | leukemia. |
| pentostatin | Nipent | Single-agent treatment for untreated hairy |
| | | cell leukemia patients with active disease as |
| | | defined by clinically significant anemia, |
| | | neutropenia, thrombocytopenia, or disease- |
| | | related symptoms. (Supplement for front - |
| | | line therapy.) |
| pipobroman | Vercyte | |
| plicamycin, | Mithracin | |
| mithramycin | | |
| porfimer sodium | Photofrin | For use in photodynamic therapy (PDT) for |
| | | palliation of patients with completely |
| | | obstructing esophageal cancer, or patients |
| | | with partially obstructing esophageal cancer |
| | | who cannot be satisfactorily treated with ND- |
| | | YAG laser therapy. |
| porfimer sodium | Photofrin | For use in photodynamic therapy for |
| | | treatment of microinvasive endobronchial |
| | | nonsmall cell lung cancer in patients for |
| | | whom surgery and radiotherapy are not |
| | | indicated. |
| porfimer sodium | Photofrin | For use in photodynamic therapy (PDT) for |
| | | reduction of obstruction and palliation of |
| | | symptoms in patients with completely or |
| | | partially obstructing endobroncial nonsmall |
| | | cell lung cancer (NSCLC). |
| procarbazine | Matulane | |
| quinacrine | Atabrine | |
| Rasburicase | Elitek | ELITEK is indicated for the initial |
| | | management of plasma uric acid levels in |
| | | pediatric patients with leukemia, lymphoma, |
| | | and solid tumor malignancies who are |
| | | receiving anti-cancer therapy expected to |
| | | result in tumor lysis and subsequent elevation |
| | | of plasma uric acid. |
| Rituximab | Rituxan | |
| Sargramostim | Prokine | |
| streptozocin | Zanosar | Antineoplastic agent. |
| talc | Sclerosol | For the prevention of the recurrence of |
| | | malignant pleural effusion in symptomatic |
| | | patients. |
| tamoxifen | Nolvadex | As a single agent to delay breast cancer |
| | | recurrence following total mastectomy and |
| | | axillary dissection in postmenopausal women |
| | | with breast cancer (T1-3, N1, M0) |
| tamoxifen | Nolvadex | For use in premenopausal women with |
| | | metastatic breast cancer as an alternative to |
| | | oophorectomy or ovarian irradiation |
| tamoxifen | Nolvadex | For use in women with axillary node- |
| | | negative breast cancer adjuvant therapy. |
| tamoxifen | Nolvadex | Metastatic breast cancer in men. |
| tamoxifen | Nolvadex | Equal bioavailability of a 20 mg Nolvadex |
| | | tablet taken once a day to a 10 mg Nolvadex |
| | | tablet taken twice a day. |
| tamoxifen | Nolvadex | to reduce the incidence of breast cancer in |
| | | women at high risk for breast cancer |
| tamoxifen | Nolvadex | In women with DCIS, following breast |
| | | surgery and radiation, Nolvadex is indicated |
| | | to reduce the risk of invasive breast cancer. |
| teniposide, VM-26 | Vumon | In combination with . other approved |
| | | anticancer agents for induction therapy in |
| | | patients with refractory childhood acute |
| | | lymphoblastic leukemia (all). |
| testolactone | Teslac | |
| thioguanine, 6-TG | Thioguanine | |
| thiotepa | Thioplex | |
| topotecan | Hycamtin | Treatment of patients with metastatic |
| | | carcinoma of the ovary after failure of initial |
| | | or subsequent chemotherapy. |
| topotecan | Hycamtin | Treatment of small cell lung cancer sensitive |
| | | disease after failure of first-line |
| | | chemotherapy. In clinical studies submitted |
| | | to support approval, sensitive disease was |
| | | defined as disease responding to |
| | | chemotherapy but subsequently progressing |
| | | at least 60 days (in the phase 3 study) or at |
| | | least 90 days (in the phase 2 studies) after |
| | | chemotherapy |
| toremifene | Fareston | Treatment of advanced breast cancer in |
| | | postmenopausal women. |
| Trastuzumab | Herceptin | HERCEPTIN as a single agent is indicated |
| | | for the treatment of patients with metastatic |
| | | breast cancer whose tumors overexpress the |
| | | HER2 protein and who have received one or |
| | | more chemotherapy regimens for their |
| | | metastatic disease. |
| Trastuzumab | Herceptin | Herceptin in combination with paclitaxel is |
| | | indicated for treatment of patients with |
| | | metastatic breast cancer whose tumors |
| | | overexpress the HER-2 protein and had not |
| | | received chemotherapy for their metastatic |
| | | disease |
| tretinoin, ATRA | Vesanoid | Induction of remission in patients with acute |
| | | promyelocytic leukemia (APL) who are |
| | | refractory to or unable to tolerate |
| | | anthracycline based cytotoxic |
| | | chemotherapeutic regimens. |
| Uracil Mustard | Uracil Mustard | |
| | Capsules | |
| valrubicin | Valstar | For intravesical therapy of BCG-refractory |
| | | carcinoma in situ (CIS) of the urinary bladder |
| | | in patients for whom immediate cystectomy |
| | | would be associated with unacceptable |
| | | morbidity or mortality. |
| vinblastine | Velban | |
| vincristine | Oncovin | |
| vinorelbine | Navelbine | Single agent or in combination with cisplatin |
| | | for the first-line treatment of ambulatory |
| | | patients with unresectable, advanced non- |
| | | small cell lung cancer (NSCLC). |
| vinorelbine | Navelbine | Navelbine is indicated as a single agent or in |
| | | combination with cisplatin for the first-line |
| | | treatment of ambulatory patients with |
| | | unreseactable, advanced non-small cell lung |
| | | cancer (NSCLC). In patients with Stage IV |
| | | NSCLC, Navelbine is indicated as a single |
| | | agent or in combination with cisplatin. In |
| | | Stage III NSCLC, Navelbine is indicated in |
| | | combination with cisplatin. |
| zoledronate | Zometa | the treatment of patients with multiple |
| | | myeloma and patients with documented bone |
| | | metastases from solid tumors, in conjunction |
| | | with standard antineoplastic therapy. Prostate |
| | | cancer should have progressed after treatment |
| | | with at least one hormonal therapy |

### Formulation and Administration:

The compounds of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

In some embodiments, provided herein a pharmaceutical composition comprises a compound of Formula IA, I, II, III, IV, a compound selected from compounds 4b, 4c, 4d, 4e, 4f, and 4g, or a pharmaceutically acceptable salt thereof.

In another embodiment, the pharmaceutical composition of this invention is for use in treating a condition in which modulating an activity of Pin1 is beneficial. In another embodiment, wherein said condition is a proliferative disease or disorder and/or an immune disease or disorder. In another embodiment, the proliferative disease or disorder is a cancer.

In another embodiment, said proliferative disease or disorder is a selected from the group consisting of a pancreatic cancer, a neuroblastoma, a prostate cancer, an ovarian carcinoma, and a breast adenocarcinoma.

In some embodiment, this invention is directed to a method for treating a condition in which modulating an activity of Pin1 is beneficial with a pharmaceutical composition of this invention. In another embodiment, said condition is a proliferative disease or disorder and/or an immune disease or disorder.

Herein the term “active ingredient” refers to one or more compounds (according to any of the respective embodiments described herein) accountable for the biological effect.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs, as well as exemplary pharmaceutically acceptable carriers, may be found “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the blood-brain barrier, “BBB”) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, optionally in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinyl pyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multi-dose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water-based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g., a compound according to any of the respective embodiments described herein, optionally in combination with an additional agent described herein) effective to prevent, alleviate or ameliorate symptoms of a disorder (e.g., a proliferative disease or disorder) or prolong the survival of the subject being treated.

Determination of a therapeutically effective amount is well within the capability of those skilled in the art, especially in light of the detailed disclosure provided herein. For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1, which is incorporated herein by reference).

Dosage amount and interval may be adjusted individually to provide levels (e.g., blood levels) of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed herein.

### Additional Definitions

### Pharmaceutically Acceptable Salt

The invention includes “pharmaceutically acceptable salts” of the compounds of this invention, which may be produced, by reaction of a compound of this invention with an acid or base. Certain compounds, particularly those possessing acid or basic groups, can also be in the form of a salt, optionally a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

Suitable pharmaceutically acceptable salts of amines of compounds the compounds of this invention may be prepared from an inorganic acid or from an organic acid. In various embodiments, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In various embodiments, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In various embodiments, examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

In some embodiments, examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In various embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

Substituted alkyl, alkenyl, alkynyl, cycloalkyl, heterocyclyl, aryl, and heteroaryl may have one or more substituents, whereby each substituent group can independently be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, amino halogen, alkylaryloxy, heteroaryloxy, oxo, cycloalkyl, phenyl, heteroaryls, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfoneamido, sulfinyl, sulfinylamino, thiol, alkylthio, arylthio, or alkylsulfonyl groups. Any substituents can be unsubstituted or further substituted with any one of these aforementioned substituents.

In some embodiments, unless otherwise specified, a substituted alkyl may be substituted with one or more (e.g., one, two, three, or more, as valency allows) groups independently selected from halo, hydroxy, alkoxy, cyano, and oxo. In some embodiments, a substituted alkyl may be substituted with one or more (e.g., one, two, three, or more, as valency allows) groups independently selected from halo and cyano.

Herein, the term “alkenyl” describes an unsaturated aliphatic hydrocarbon comprise at least one carbon-carbon double bond, including straight chain and branched chain groups. Optionally, the alkenyl group has 2 to 20 carbon atoms. More optionally, the alkenyl is a medium size alkenyl having 2 to 10 carbon atoms. Most optionally, unless otherwise indicated, the alkenyl is a lower alkenyl having 2 to 4 carbon atoms. The alkenyl group may be substituted or non-substituted.

Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfoneamido, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

In some embodiments, unless otherwise specified, a substituted alkenyl may be substituted with one or more (e.g., one, two, three, or more, as valency allows) groups independently selected from halo, hydroxy, alkoxy, cyano, and oxo. In some embodiments, a substituted alkenyl may be substituted with one or more (e.g., one, two, three, or more, as valency allows) groups independently selected from halo and cyano.

Herein, the term “alkynyl” describes an unsaturated aliphatic hydrocarbon comprise at least one carbon-carbon triple bond, including straight chain and branched chain groups. Optionally, the alkynyl group has 2 to 20 carbon atoms. More optionally, the alkynyl is a medium size alkynyl having 2 to 10 carbon atoms. Most optionally, unless otherwise indicated, the alkynyl is a lower alkynyl having 2 to 4 carbon atoms. The alkynyl group may be substituted or non-substituted.

Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

In some embodiments, unless otherwise specified, a substituted alkynyl may be substituted with one or more (e.g., one, two, three, or more, as valency allows) groups independently selected from halo, hydroxy, alkoxy, cyano, and oxo. In some embodiments, a substituted alkynyl may be substituted with one or more (e.g., one, two, three, or more, as valency allows) groups independently selected from halo and cyano.

As used herein, “alkylene” refers to a linear, branched or cyclic, in certain embodiments linear or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms including —NR— and —N⁺RR-groups, where the nitrogen substituent(s) is(are) alkyl, aryl, aralkyl, heteroaryl, heteroaralkyl or COR, wherein each R is independently selected from alkyl, aryl, aralkyl, heteroaryl, heteroaralkyl, —OY or —NYY, wherein each Y is independently selected from hydrogen, alkyl, aryl, heteroaryl, cycloalkyl or heterocyclyl. Alkylene groups include, but are not limited to, methylene (—CH₂), ethylene (—CH₂CH₂—), propylene (—(CH₂)₃), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “alkenylene” refers to a linear, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —H═CH—CH₂. The term “lower alkenylene” refers to alkenylene groups having 2 to 6 carbons. In certain embodiments, alkenylene groups are lower alkenylene, including alkenylene of 3 to 4 carbon atoms.

In some embodiments, unless otherwise specified, a substituted alkenylene may be substituted with one or more (e.g., one, two, three, or more, as valency allows) groups independently selected from halo, hydroxy, alkoxy, cyano, and oxo. In some embodiments, a substituted alkenylene may be substituted with one or more (e.g., one, two, three, or more, as valency allows) groups independently selected from halo and cyano.

As used herein, “alkynylene” refers to a straight, branched or cyclic, in certain embodiments straight or branched, a divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C≡C, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

In some embodiments, unless otherwise specified, a substituted alkynylene may be substituted with one or more (e.g., one, two, three, or more, as valency allows) groups independently selected from halo, hydroxy, alkoxy, cyano, and oxo. In some embodiments, a substituted alkynylene may be substituted with one or more (e.g., one, two, three, or more, as valency allows) groups independently selected from halo and cyano.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond. The cycloalkyl group can be an end group, as this phrase is defined herein, wherein it is attached to a single adjacent atom, or a linking group, as this phrase is defined herein, connecting two or more moieties.

In some embodiments, unless otherwise specified, a substituted cycloalkyl may be substituted with one or more (e.g., one, two, three, or more, as valency allows) groups independently selected from halo, hydroxy, alkoxy, cyano, and oxo. In some embodiments, a substituted cycloalkyl may be substituted with one or more (e.g., one, two, three, or more, as valency allows) groups independently selected from halo and cyano.

An “aryl” group refers to an all-carbon monocyclic or fused-ring polycyclic (i.e., rings which share adjacent pairs of carbon atoms) end groups having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein. The aryl group can be an end group, as this phrase is defined herein, wherein it is attached to a single adjacent atom, or a linking group, as this phrase is defined herein, connecting two or more moieties.

In some embodiments, unless otherwise specified, a substituted aryl may be substituted with one or more (e.g., one, two, three, or more, as valency allows) groups independently selected from halo, hydroxy, alkoxy, cyano, and oxo. In some embodiments, a substituted aryl may be substituted with one or more (e.g., one, two, three, or more, as valency allows) groups independently selected from halo and cyano.

A “heteroaryl” group refers to a monocyclic or fused ring (i.e., rings which share an adjacent pair of atoms) end group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein.

In some embodiments, unless otherwise specified, a substituted heteroaryl may be substituted with one or more (e.g., one, two, three, or more, as valency allows) groups independently selected from halo, hydroxy, alkoxy, cyano, and oxo. In some embodiments, a substituted heteroaryl may be substituted with one or more (e.g., one, two, three, or more, as valency allows) groups independently selected from halo and cyano.

The term “arylene” describes a monocyclic or fused-ring polycyclic linking group, as this term is defined herein, and encompasses linking groups which differ from an aryl or heteroaryl group, as these groups are defined herein, only in that arylene is a linking group rather than an end group.

A “heterocyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like. The heteroalicyclic group can be an end group, as this phrase is defined herein, wherein it is attached to a single adjacent atom, or a linking group, as this phrase is defined herein, connecting two or more moieties.

In some embodiments, unless otherwise specified, a substituted heterocyclic may be substituted with one or more (e.g., one, two, three, or more, as valency allows) groups independently selected from halo, hydroxy, alkoxy, cyano, and oxo. In some embodiments, a substituted heterocyclic may be substituted with one or more (e.g., one, two, three, or more, as valency allows) groups independently selected from halo and cyano.

Herein, the terms “amine” and “amino” each refer to either a —NR′R″ end group, a —N⁺R′R″R′″ end group, a —NR′-linking group, or a —N⁺R′R″-linking group, wherein R′, R″ and R′″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R′″ are hydrogen or alkyl comprising 1 to 4 carbon atoms. Optionally, R′ and R″ (and R′″, if present) are hydrogen. When substituted, the carbon atom of an R′, R″ or R′″ hydrocarbon moiety which is bound to the nitrogen atom of the amine is optionally not substituted by oxo, such that R′, R″ and R′″ are not (for example) carbonyl, C-carboxy or amide, as these groups are defined herein, unless indicated otherwise.

An “azide” group refers to a —N═N⁺═N⁻ group.

An “alkoxy” group refers to both an —O-alkyl and an —O-cycloalkyl end group, as defined herein, or to an —O-alkylene- or —O-cycloalkyl-linking group, as defined herein.

An “aryloxy” group refers to both an —O-aryl and an —O-heteroaryl end group, as defined herein, or to an —O-arylene-linking group, as defined herein.

A “hydroxy” group refers to a —OH group.

A “thiohydroxy” or “thiol” group refers to a —SH group.

A “thioalkoxy” group refers to both an —S-alkyl end group and an —S-cycloalkyl end group, as defined herein, or to an —S-alkylene- or —S-cycloalkyl-linking group, as defined herein.

A “thioaryloxy” group refers to both an —S-aryl and an —S-heteroaryl end group, as defined herein, or to an —S-arylene-linking group, as defined herein.

A “carbonyl” group refers to a —C(═O)—R′ end group, where R′ is defined as hereinabove, or to a —C(═O)-linking group.

A “thiocarbonyl” group refers to a —C(═S)—R′ end group, where R′ is as defined herein, or to a —C(═S)-linking group.

A “carboxyl”, “carboxylic” or “carboxylate” refers to both “C-carboxy” and O-carboxy” end groups, as well as to a —C(═O)—O-linking group.

A “C-carboxy” group refers to a —C(═O)—O—R′ group, where R′ is as defined herein.

An “O-carboxy” group refers to an R′C(═O)—O-group, where R′ is as defined herein.

A “carboxylic acid” refers to a —C(═O)OH group, including the deprotonated ionic form and salts thereof.

An “ester” refers to a —C(═O)OR′ or —O—(C═O)R′ group, wherein R′ is not hydrogen.

An “oxo” group refers to a ═O group.

A “thiocarboxy” or “thiocarboxylate” group refers to both —C(═S)—O—R′ and —O—C(═S)R′ end groups, or to a —C(═S)—O-linking group, wherein R′ is not hydrogen.

A “halo” group refers to fluorine, chlorine, bromine or iodine.

A “haloalkyl” group refers to an alkyl group substituted by one or more halo groups, as defined herein.

A “sulfinyl” group refers to an —S(═O)—R′ end group, where R′ is as defined herein, or to a —S(═O)-linking group.

A “sulfonyl” group refers to an —S(═O)₂—R′ end group, where R′ is as defined herein, or to a —S(═O)₂-linking group.

A “sulfonate” group refers to an —S(═O)₂—O—R′ end group, where R′ is as defined herein, or to a S(═O)₂—O-linking group.

A “sulfate” group refers to an —O—S(═O)₂—O—R′ end group, where R′ is as defined as herein, or to a —O—S(═O)₂—O-linking group.

A “sulfonamide” or “sulfonamido” group encompasses both S-sulfonamido and N-sulfonamido end groups, as defined herein, and a —S(═O)₂—NR′-linking group.

An “S-sulfonamido” group refers to a —S(═O)₂—NR′R″ group, with each of R′ and R″ as defined herein.

An “N-sulfonamido” group refers to an R'S(═O)₂—NR″ group, where each of R′ and R″ is as defined herein.

A “carbamyl” or “carbamate” group encompasses O-carbamyl and N-carbamyl end groups, and to a —OC(═O)—NR′-linking group.

An “O-carbamyl” group refers to an —OC(═O)—NR′R″ group, where each of R′ and R″ is as defined herein.

An “N-carbamyl” group refers to an R′OC(═O)—NR″-group, where each of R′ and R″ is as defined herein.

A “thiocarbamyl” or “thiocarbamate” group encompasses O-thiocarbamyl and N-thiocarbamyl end groups, and to a —OC(═S)—NR′-linking group.

An “O-thiocarbamyl” group refers to an —OC(═S)—NR′R″ group, where each of R′ and R″ is as defined herein.

An “N-thiocarbamyl” group refers to an R′OC(═S)NR″-group, where each of R′ and R″ is as defined herein.

An “amide” or “amido” group encompasses C-amido and N-amido end groups, as defined herein, and to a —C(═O)—NR′-linking group.

A “C-amido” group refers to a —C(═O)—NR′R″ group, where each of R′ and R″ is as defined herein.

An “N-amido” group refers to an R′C(═O)—NR″-group, where each of R′ and R″ is as defined herein.

A “urea group” refers to an —N(R′)—C(═O)—NR″R′″ end group, or to a N(R′)—C(═O)—NR″-linking group, where each of R′, R″ and R″ is as defined herein.

A “thiourea group” refers to a —N(R′)—C(═S)—NR″R′″ end group, or to a —N(R′)—C(═S)—NR″-linking group where each of R′, R″ and R″ is as defined herein.

A “nitro” group refers to an —NO₂ group.

A “cyano” group refers to a —C—N group.

The term “phosphonyl” or “phosphonate” describes a —P(═O)(OR′)(OR″) end group, or a —P(═O)(OR′)—O-linking group, with R′ and R″ as defined hereinabove.

The term “phosphate” describes an —O—P(═O)(OR′)(OR″) end group, or a —O—P(═O)(OR′)—O-linking group with each of R′ and R″ as defined hereinabove.

The term “phosphinyl” describes a —PR′R″ end group, or —PRR′-linking group, with each of R′ and R″ as defined hereinabove.

The term “hydrazine” describes a —NR′—NR″R′″ end group, or —NR′—NR″-linking group, with R′, R″, and R′″ as defined herein.

As used herein, the term “hydrazide” describes a —C(═O)—NR′—NR″R′″ end group, or —C(═O)—NR′—NR″-linking group, where R′, R″ and R′″ are as defined herein.

As used herein, the term “thiohydrazide” describes a —C(═S)—NR′—NR″R′″ end group, or —C(═S)—NR′—NR″-linking group, where R′, R″ and R′″ are as defined herein.

A “guanidinyl” group refers to an —RₐNC(═NR_{d})—NR_{b}R_{c} end group, or —RₐNC(═NR_{d})—NRb-linking group where each of Rₐ, R_{b}, R_{c} and R_{d} can be as defined herein for R′ and R″.

A “guanyl” or “guanine” group refers to an RₐR_{b}NC(═NR_{d})— end group, or a —RₐNC(═NR_{d})-linking group, where Rₐ, R_{b} and R_{d} are as defined herein.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

In some embodiments provided herein a method of this invention for treating immune disease or disorders with the compounds disclosed herein, specifically Sulfamate compounds. An immune disease or disorder refers herein to an autoimmune disease. In other embodiments to autoimmune disease selected from: Addison disease, Celiac disease—sprue (gluten-sensitive enteropathy), Dermatomyositis, Graves disease, Hashimoto thyroiditis, Multiple sclerosis, Myasthenia gravis, Pernicious anemia, Reactive arthritis, Rheumatoid arthritis, Sjögren syndrome, Systemic lupus erythematosus and Type I diabetes.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Methods

### LC/MS Measurements

LC/MS runs were performed on a Waters ACQUITY UPLC class H instrument, in positive ion mode using electrospray ionization. UPLC separation for small molecules used C18-CSH column of (1.7 μm, 2.1 mm×100 mm) for all the LC/MS based assays. The column was held at 40° C. and the autosampler at 10° C. Mobile phase A was 0.1% formic acid in the water, and mobile phase B was 0.1% formic acid in acetonitrile. The run flow was 0.3 mL/min. The gradient used was 100% A for 2 min, increasing linearly to 90% B for 5 min, holding at 90% B for 1 min, changing to 0% B in 0.1 min, and holding at 0% for 1.9 min. UPLC separation for proteins used a C4 column (300 Å, 1.7 μm, 2.1 mm×100 mm). The column was held at 40° C. and the autosampler at 10° C. Mobile solution A was 0.1% formic acid in the water, and mobile phase B was 0.1% formic acid in acetonitrile. The run flow was 0.4 mL/min with gradient 20% B for 2 min, increasing linearly to 60% B for 3 min, holding at 60% B for 1.5 min, changing to 0% B in 0.1 min, and holding at 0% for 1.4 min. The mass data were collected on a Waters SQD2 detector with an m/z range of 2-3071.98 at a range of m/z of 800-1500 Da for BTK, and 750-1550 for Pin1.

GSH Reactivity Assay for Compounds 1a-1h (Model Compounds)

A 200 μM (for 1a-1h) (0.5 or 1 μL of 20 mM stock) sample of the electrophile was incubated with 5 mM GSH (5 μL of 100 mM stock, freshly dissolved), 5 mM NaOH (to counter the acidity imparted by GSH) and 100 μM 4-nitrocyano benzene (0.5 μl of 20 mM stock solution) as an internal standard in 100 mM potassium phosphate buffer pH 8.0 and DMF at a ratio of 9:1, respectively. All solvents were bubbled with argon. Reaction mixtures were kept at 10° C. Every 1 h, 5 μL from the reaction mixture were injected into the LC/MS. The reaction was followed by the peak area of the electrophile normalized by the area of the 4-nitrocyano benzene (i.e. by the disappearance of the starting material). The natural logarithm of the results was fitted to linear regression, and t_{1/2} was calculated as t_{1/2}=ln 2/−slope.

### DTNB Thiol Reactivity Assay

The compound 5,5′-dithio-bis(2-nitrobenzoic acid) (DTNB; 50 μM) was incubated with 200 μM tris(2-carboxyethyl)phosphine (TCEP) in 20 mM sodium phosphate buffer pH 7.4 and 150 mM NaCl for 5 min at room temperature, to obtain TNB⁻². Next, 200 μM compounds were subsequently added to TNB⁻² followed by immediate ultraviolet (UV) absorbance measurement at 412 nm and 37° C. UV absorbance was acquired every 15 min for 7 h. The assay was performed in a 384-well plate using a Tecan Spark 10M plate reader. Background absorbance of compounds was subtracted by measuring the absorbance at 412 nm of each compound under the same conditions without DTNB. Compounds were measured in triplicate. The data were fitted to a second-order reaction equation such that the rate constant (K) is the slope of ln([A][B0]/[B][A0]), where [A0] and [B0] are the initial concentrations of the compound (200 μM) and TNB⁻² (100 μM), respectively, and [A] and [B] are the remaining concentrations as a function of time as deduced from spectrometric measurements. Linear regression using Prism was performed to fit the rate against the first 7 h of measurements.

### Buffer Stability Assay for Model Compound, Ibrutinib Sulfamates and Sulfamate Compounds

A sample of the electrophile (200 μM for 1a-1i, and 4a-4g) was incubated with 100 μM of 4-nitrocyano benzene as an internal standard in a 100 mM potassium phosphate buffer of pH 8.0. Reaction mixtures were kept at 37° C. with shaking. After 4 days (unless otherwise mentioned), 5 μL from the reaction mixture were injected into the LC/MS to check the stability of the compounds.

### In-Vitro Labeling Experiments

Pin1 was expressed and purified as previously reported³⁸. The catalytic domain of Pin1 (2 μM) in 20 mM Tris, 75 mM NaCl, pH 7.5 was incubated with 2 μM Sulfamate (4a-4g) for 1 h at 25° C.

The reaction mixtures, at room temperature for various times, were injected into the LC/MS. For data analysis, the raw spectra were deconvoluted using a 20000:40000 Da (For Pin1, 10000:30000) window and 1 Da resolution. The labeling percentage for a compound was determined as the labeling of a specific compound (alone or together with other compounds) divided by the overall detected protein species.

### Pin1 Pull-Down Using Sulfopin-DTB Probe

OCI-AML2 cells were treated for 4 h with either DMSO (0.1%) or Sulfopin, 4c, 4d, 4e and 4g (0.5, 2.5 mM). The cells were lysed using RIPA buffer (Sigma, R0278). Lysates were clarified at 21,000 g for 15 minutes at 4° C. and protein concentration was determined using BCA protein assay (Thermo Scientific, 23225). Lysates were incubated with 1 mM Sulfopin-DTB probe for 1 h at room-temperature, using 650 mg per sample. Streptavidin-agarose beads (Thermo Scientific, 20349) were added, 50 ul per sample, and placed on a shaker for 2 h in room temperature. The beads were washed four times with 500 ml buffer containing 20 mM Hepes pH 7.5, 10 mM NaCl, 1 mM EDTA, 10% glycerol. Beads were then pelleted and boiled in 50 ml 2×LDS sample buffer (Invitrogen, NuPAGE, NP0007) and Pin1 immunoblotting was performed. The samples were loaded on a 4-20% Bis-Tris gel (SurePAGE) and transferred to a nitrocellulose membrane (Bio-Rad, 1704158) using Trans-Blot Turbo system (Bio-Rad). The membrane was blocked using 5% BSA in PBS-T (w/v) for 1 hour at room temperature, washed ×3 times for 5 minutes with PBS-T and incubated overnight at 4° C. with Pin1 antibody diluted 1:1,000 (Cell Signaling, 3722). Membrane was washed ×3 times for 5 minutes with PBS-T and incubated with anti-rabbit HRP-linked secondary antibody (cell-signaling, #7074) for 1 hour at room-temperature. EZ-ECL Kit (Biological Industries, 20-500-1000) was used to detect HRP-activity.

### IsoTOP ABPP Sample Preparation

The preparation of IsoTOP-ABPP samples was performed essentially as described in Zanon et al. (Zanon, P. R. A., Lewald, L. & Hacker, S. M. Angew. Chem. Int. Ed Engl. 59 which is incorporated herein by reference).

Experiments were conducted in quadruplicates. PATU cells were incubated for 2 h with 5 μM compounds Sulfopin (or with DMSO), and collected by centrifuge at 300 g for 5 min followed by ice cold PBS wash. For lysis, samples containing 10 million cells were dispersed in 0.5 mL of RIPA buffer (Sigma, R0278), incubated with occasional vortexing for 30 min on ice, followed by centrifugation at 21,000 g for 15 min. The protein concentration in the samples was determined using BCA assay (Pierce 23227), and each sample was diluted to 1.7 mg/mL using PBS. To each sample, 5 μL of 10 mM IA-alkyne was added, followed by 1 h incubation at room temperature in the dark. 10 μL of 5 mM DesThioTag was added (Light for the compound treated samples, heavy for the DMSO-treated samples), followed by 18 μL of CuSO4:THPTA (100 mM), and the click reaction was initiated by addition of 15 μL of 150 mM sodium ascorbate (freshly dissolved in water). The samples were incubated on a rotary shaker for 1 h at room temperature. The compound-treated and DMSO-treated samples were mixed with 4 mL methanol, 1 mL chloroform and 2 mL water on ice, vortexed and centrifuged at 3200 g for 10 min at 4° C. The top layer was aspirated, and 3 mL methanol was added, followed by centrifugation and aspiration of the supernatant. The pellets were air dried and stored at −80° C. until the following treatment. The pellets were resuspended in 0.3 mL of 8 M urea freshly dissolved in PBS using probe sonication (8 sec total at 40% amplitude, 1 sec on/2 sec off, at room temperature). Following the resuspension, the samples were diluted with 1 mL of PBS. Then each sample was mixed with 1.3 mL of slurry containing 110 μL of streptavidin agarose beads (Thermo Streptavidin Agarose cat #20349), prewashed, and dispersed in 0.2% IGEPAL. The samples were incubated with rotation for 3 h at room temperature. The beads were pelleted by centrifugation at 2000 g for 2 min, transferred to spin columns, and washed 3 times with 0.1% IGEPAL/PBS, 3 times PBS, and 3 times water. The beads were then suspended in 8 M Urea/50 mM ammonium bicarbonate, and 15 μL of 31 mg/mL DTT were added, followed by incubation at 37° C. for 45 min. The samples were cooled to room temperature, and 15 μL of 74 mg/mL iodoacetamide were added, followed by 30 min incubation at room temperature in the dark, and the addition of a further 15 μL of 31 mg/mL DTT and incubation at room temperature for 30 min. 900 μL of 50 mM ammonium bicarbonate were added, and after 30 min incubation, the beads were pelleted by centrifugation at 2000 g for 2 min, and resuspended in 200 μL of 1 M Urea/50 mM ammonium bicarbonate. At this point, modified trypsin (Promega V511A) was dissolved in trypsin buffer at 0.5 μg/μL, and 4 μL were added to each sample, followed by overnight incubation at 37° C. with shaking. 400 μL of 0.1% IGEPAL/PBS were added, and the beads were washed 3 times with 0.1% IGEPAL/PBS, 3 times PBS and 3 times water. The peptides were eluted by incubation with 200 μL of 50% acetonitrile+0.1% TFA for 5 min, followed by two more portions of 100 μL of 50% acetonitrile+0.1% TFA. The samples were dried by speedvac, and further purified using Oasis desalting columns (Waters), after which they were dried and run on the instrument.

### Running Proteomics Samples

Samples were analyzed using EASY-nLC 1200 nano-flow UPLC system, using PepMap RSLC C18 column (2 m particle size, 100 Å pore size, 75 μm diameter×50 cm length), mounted using an EASY-Spray source onto an Exploris 240 mass spectrometer. uLC/MS-grade solvents were used for all chromatographic steps at 300 nL/min. The mobile phase was: (A) H2O+0.1% formic acid and (B) 80% acetonitrile+0.1% formic acid. Each sample (2 μL) was injected. Peptides were eluted from the column into the mass spectrometer using the following gradient: 1-40% B in 160 min, 40-100% B in 5 min, maintained at 100% for 20 min, 100 to 1% in 10 min, and finally 1% for 5 min. Ionization was achieved using a 1900 V spray voltage with an ion transfer tube temperature of 275° C. Data were acquired in data-dependent acquisition (DDA) mode. MS1 resolution was set to 120,000 (at 200 m/z), a mass range of 375-1650 m/z, normalized AGC of 300%, and the maximum injection time was set to 20 ms. MS2 resolution was set to 15,000, quadrupole isolation 1.4 m/z, normalized AGC of 50%, dynamic exclusion of 45 s, and automatic maximum injection time.

### Proteomics Data Analysis

Data analysis for pull down samples was performed using MaxQuant 1.6.3.4. Human proteome (updated November 2020) was downloaded from Uniprot. Carbamidomethyl was used as a fixed modification and methionine oxidation and N terminal acetylation as variable modification. The digestion enzyme was set to Trypsin/P with a maximum number of missed cleavages of 2. The “Re-quantify” option was enabled. Contaminants were included. Peptides were searched with a minimum peptide length of 7 and a maximum peptide mass of 4,600 Da. “Second peptides” was enabled and “Dependent peptides” were disabled. The option “Match between run” was enabled with a Match time window of 0.7 min and an alignment window of 20 min. An FDR of 0.01 was used for Protein FDR, PSM FDR and XPSM FDR. Label free quantification was used to quantify the proteins, with each data set (DMSO-treated samples, compound-treated samples, etc.) analyzed with a separate parameter group. The results were analyzed with Perseus. Contaminant proteins, proteins only identified by modified peptides, and proteins identified from reverse peptides were removed. LFQ intensities were transformed into Log 2 values and only proteins in which at least one data set contained 3 non zero intensities (out of 4) were retained. Non-valid values were replaced by values from a normal distribution with a width of 0.3 sigma and a down shift of 1.8 sigma. The data was finally analyzed using students T-test.

Data analysis for IsoTOPP data Analysis of IsoTOP-ABPP data was performed similarly to (Zanon, P. R. A., Lewald, L. & Hacker, S. M. Angew. Chem. Int. Ed Engl. 59, 2829-2836 (2020), which is hereby incorporated by reference in its entirety) using MaxQuant 1.6.3.4. Human proteome (updated November 2020) was downloaded from Uniprot. For each protein not containing selenocysteine, a copy of the protein sequence containing a single mutation of cysteine to selenocysteine (C→U) was created for each cysteine in the sequence to an unmutated copy. The IsoTOPP labels were then defined as Heavy/Light labels with the following formulae: C(24)H(49)N(8)Cx(5)Nx(1)S(1)Se(−1) for the heavy label, and C(29)H(49)N(9)S(1)Se(−1) for the light label. In addition diagnostic peaks were added corresponding to the free amine generated by cleavage of the iodoacetamide alkyne (C(22)H(49)N(8)O(4)Cx(5)Nx(1)/C(27)H(49)N(9)O(4)), internal cleavage caused by attack of the triazole on the alpha carbon of the iodoacetamide moiety² (C(22)H(46)N(7)O(4)Cx(5)Nx(1)/C(27)H(46)N(8)O(4)), cleavage of the peptide bond between azidolysine and valine (C(10)H(25)N(2)O(3)Cx(5)Nx(1)/C(15)H(25)N(3)O(3)), and cleavage of the peptide bond between valine and desthiobiotin (C(10)H(16)N(2)O(2)). A multiplicity of 2 was set, and maximum number of labeled amino acids of 1. The digestion enzyme was set to Trypsin/P with a maximum number of missed cleavages of 2. No variable modifications were included. The “Re-quantify” option was enabled. Carbamidomethyl (C2H3NO) was used as a fixed modification on cysteine. Contaminants were included. Peptides were searched with a minimum peptide length of 7 and a maximum peptide mass of 4,600 Da. “Second peptides” and “Dependent peptides” were disabled and the option “Match between run” was enabled with a Match time window of 0.7 min and an alignment window of 20 min. An FDR of 0.01 was used for Protein FDR, PSM FDR and XPSM FDR. After MaxQuant analysis, the data for each compound was analyzed separately. Following data analysis, reverse and contaminant peptides were removed. Only peptides for which non-zero total intensities were measured for at least two of the replicates were analyzed. Average H/L ratios were calculated as the ratio of the sum of high intensities in the replicates to the sum of the low intensities. Ratios that were above 20 or infinite (due to the sum being zero for the low intensities) were defined as 20.

### Example 1

### Synthesis of Model Compounds

### Synthesis of N-benzyl-2-chloroacetamide (1a)

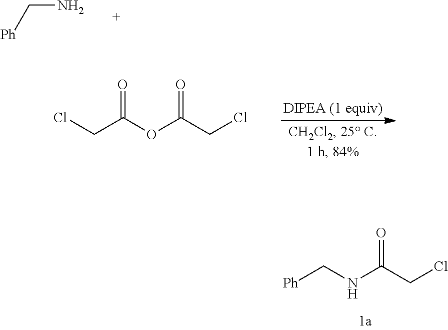 [SMILES: NCc1ccccc1 ; O=C(CCl)OC(=O)CCl ; O=C(CCl)NCc1ccccc1]

To a stirred solution of Bn-NH₂ (108 μL, 1 mmol) in anhydrous DCM (2 mL), DIPEA (178 μL, 1 mmol) and chloroacetic anhydride (170 mg, 1 mmol) were added at 25° C. The reaction mixture was stirred at room temperature for 1 h. After completion of the reaction (as monitored by LC-MS), CH₂Cl₂ was concentrated in vacuo and the crude product was purified using combi flash column chromatography using EtOAc:Hexane as eluent to give 1a in 154 mg (yield=84%).

¹H NMR (500 MHz, CDCl₃): δ 4.12 (s, 2H), 4.51 (d, J 5.8 Hz, 2H), 6.88 (br. s., 1H), 7.28-7.34 (m, 3H), 7.34-7.42 (m, 2H).

¹³C NMR (126 MHz, CDCl₃): δ 42.6, 43.9, 127.8, 127.8, 128.8, 137.2, 165.8.

ESI-MS (m/z): calculated for C₉H₁₁ClNO [M+H]+: 184.05; found: [M+H]+:184.82.

### Synthesis of N-benzyl-2-hydroxyacetamide (Pre-1)

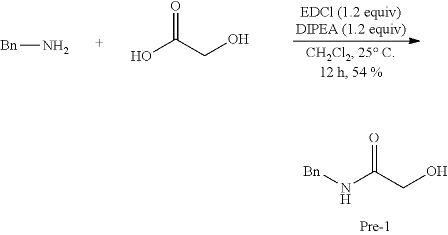 [SMILES: NCc1ccccc1 ; O=C(O)CO ; O=C(CO)NCc1ccccc1]

To a stirred solution of Bn-NH₂ (324 μL, 3 mmol) in anhydrous DCM (6 mL), 1-Ethyl-3-(3-dimethylaminopropyl)carbodiimide hydrochloride (690 mg, 3.6 mmol) and DIPEA (600 μL, 3.6 mmol) were added at 25° C. The reaction mixture was stirred at room temperature for 12 h. After completion of the reaction (as monitored by LC-MS), the reaction mixture was evaporated under vacuo and the crude product was purified using combi flash column chromatography with MeOH:EtOAc (2:8) as eluent to give Pre-1 was colorless solid in 267 mg (yield=54%).

ESI-MS (m/z): calculated for C₉H₁₂NO₂ [M+H]+: 166.08; found: [M+H]+:166.30.

### Synthesis of 2-(benzylamino)-2-oxoethyl Methanesulfonate (1b)

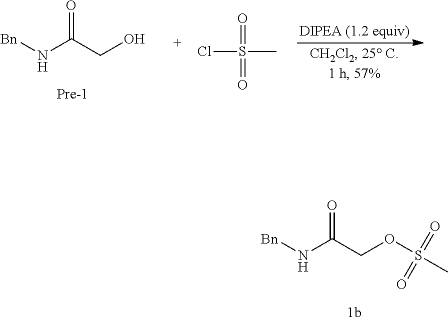

To a stirred solution of Pre-1 (16.5 mg, 0.1 mmol) in CH₂Cl₂ (1 mL), methane sulfonyl chloride (9.2 μL, 0.12 mmol, d=1.48), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 1b in 13.8 mg (57% yield).

¹H NMR (400 MHz, CDCl₃) δ: 2.50 (br. s., 1H), 3.07 (s, 3H), 4.47 (d, J 5.7 Hz, 2H), 4.66 (s, 2H), 6.67 (br. s., 1H), 7.19-7.40 (m, 4H).

¹³C NMR (101 MHz, CDCl₃) δ 37.8, 43.4, 66.6, 127.8, 127.8, 128.8, 137.0, 165.2.

ESI-MS (m/z): calculated for C₁₀H₁₄NO₄S [M+H]+: 244.06; found: [M+H]+:244.64.

### Synthesis of 2-(benzylamino)-2-oxoethyl 4-methylbenzenesulfonate (1c)

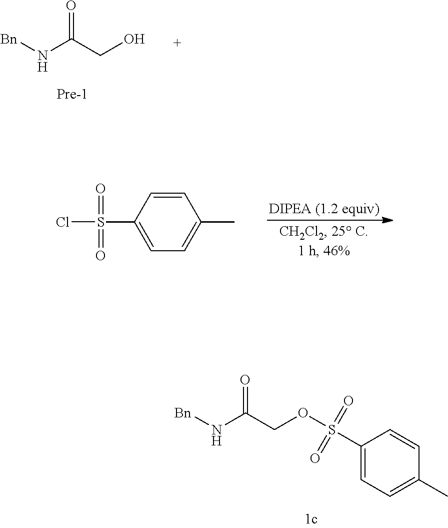 [SMILES: O=C(CO)NCc1ccccc1 ; Cc1ccc(S(=O)(=O)Cl)cc1 ; Cc1ccc(S(=O)(=O)OCC(=O)NCc2ccccc2)cc1]

To a stirred solution of Pre-1 (16.5 mg, 0.1 mmol) in CH₂Cl₂ (1 mL), 4-toluenesulfonyl chloride (22.8 mg, 0.12 mmol), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 1c in 14.6 mg (46% yield).

¹H NMR (500 MHz, CDCl₃) δ: 2.47 (s, 3H), 4.46 (d, J 5.9 Hz, 2H), 4.49 (s, 2H), 6.66 (br. s., 1H), 7.25 (d, J 7.0 Hz, 2H), 7.28-7.33 (m, 1H), 7.37 (d, J 8.1 Hz, 2H), 7.34 (d, J 7.6 Hz, 2H), 7.79 (d, J 8.3 Hz, 2H).

¹³C NMR (126 MHz, CDCl₃) δ: 21.7, 43.3, 66.9, 127.8, 128.1, 128.8, 130.2, 131.6, 137.0, 145.9, 165.2.

ESI-MS (m/z): calculated for C₁₆H₁₈NO₄S [M+H]+: 320.10; found: [M+H]+:320.55.

### Synthesis of 2-(benzylamino)-2-oxoethyl Methylsulfamate (1d)

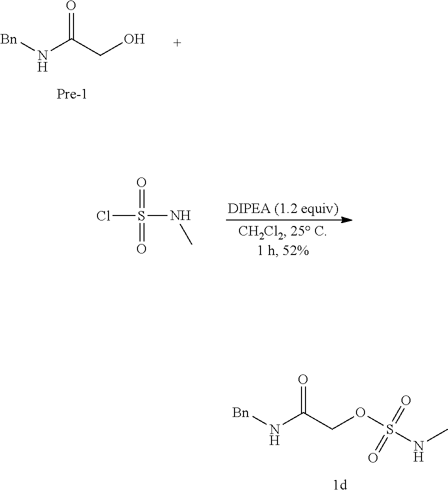

To a stirred solution of Pre-1 (16.5 mg, 0.1 mmol) in CH₂Cl₂ (1 mL), N-methylsulfamoyl chloride (15.4 mg, 0.12 mmol), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 1d in 13.4 mg (52% yield).

¹H NMR (400 MHz, CDCl₃) δ: 2.79 (d, J=5.1 Hz, 3H), 4.50 (d, J=5.7 Hz, 2H), 4.59 (s, 2H), 5.24 (d, J=4.6 Hz, 1H), 6.79 (br. s., 1H), 7.18-7.45 (m, 4H)

¹³C NMR (100 MHz, CDCl₃) δ: 29.8, 43.3, 67.2, 127.8, 128.8, 137.0, 166.0.

ESI-MS (m/z): calculated for C₁₀H₁₅N₂O₄S [M+H]+: 259.08; found: [M+H]+:259.64.

### Synthesis of 2-(benzylamino)-2-oxoethyl Benzylsulfamate (1e)

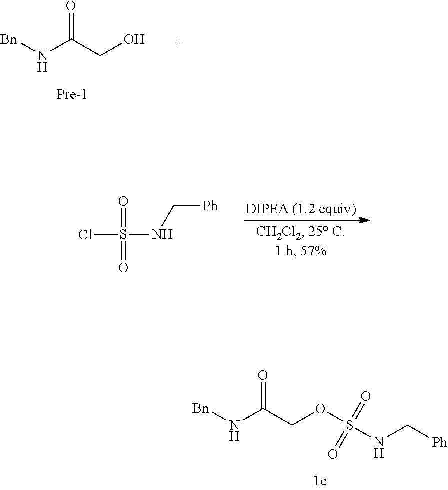 [SMILES: O=C(CO)NCc1ccccc1 ; O=S(=O)(Cl)NCc1ccccc1 ; O=C(COS(=O)(=O)NCc1ccccc1)NCc1ccccc1]

To a stirred solution of Pre-1 (16.5 mg, 0.1 mmol) in CH₂Cl₂ (1 mL), benzylsulfamoyl chloride (24 mg, 0.12 mmol), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 1e in 19 mg (57% yield).

¹H NMR (500 MHz, CDCl₃) δ: 4.31 (d, J 5.6 Hz, 2H), 4.45 (s, 2H), 4.49 (s, 2H), 5.40 (t, J 5.4 Hz, 1H), 6.48 (br. s., 1H), 7.24-7.29 (m, 3H), 7.29-7.35 (m, 6H), 7.35-7.40 (m, 2H).

¹³C NMR (126 MHz, CDCl₃) δ: 43.3, 48.0, 67.1, 127.8, 127.8, 128.1, 128.5, 128.8, 129.0, 135.7, 137.0, 165.6.

ESI-MS (m/z): calculated for C₁₆H₁₉N₂O₄S [M+H]+: 335.11; found: [M+H]+:335.18.

### Synthesis of 2-(benzylamino)-2-oxoethyl Phenylsulfamate (1f)

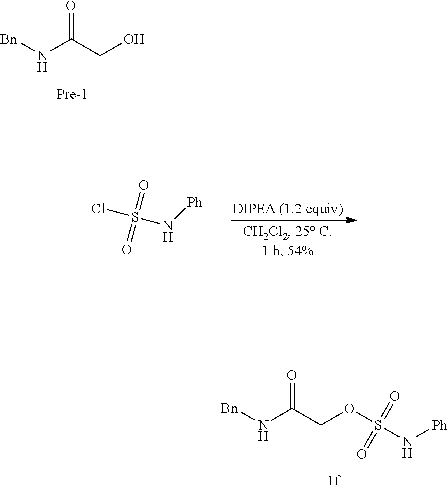 [SMILES: O=C(CO)NCc1ccccc1 ; O=S(=O)(Cl)Nc1ccccc1 ; O=C(COS(=O)(=O)Nc1ccccc1)NCc1ccccc1]

To a stirred solution of Pre-1 (16.5 mg, 0.1 mmol) in CH₂Cl₂ (1 mL), phenylsulfamoyl chloride (22.5 mg, 0.12 mmol), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid if in 17.2 mg (54% yield).

¹H NMR (400 MHz, CDCl₃) δ: 4.42 (d, J 5.7 Hz, 2H), 4.66 (s, 2H), 6.55 (br. s., 1H), 7.21 (d, J 7.5 Hz, 4H), 7.33 (d, J 7.7 Hz, 4H), 7.49 (s, 1H).

¹³C NMR (126 MHz, CDCl₃) δ: 29.3, 42.7, 66.6, 119.7, 121.0, 124.7, 126.8, 127.3, 128.3, 129.1, 135.9, 162.4 (s).

ESI-MS (m/z): calculated for C₁₅H₁₇N₂O₄S [M+H]+: 321.09; found: [M+H]+:321.57.

### Synthesis of 2-(benzylamino)-2-oxoethyl (4-bromophenyl)sulfamate (1g)

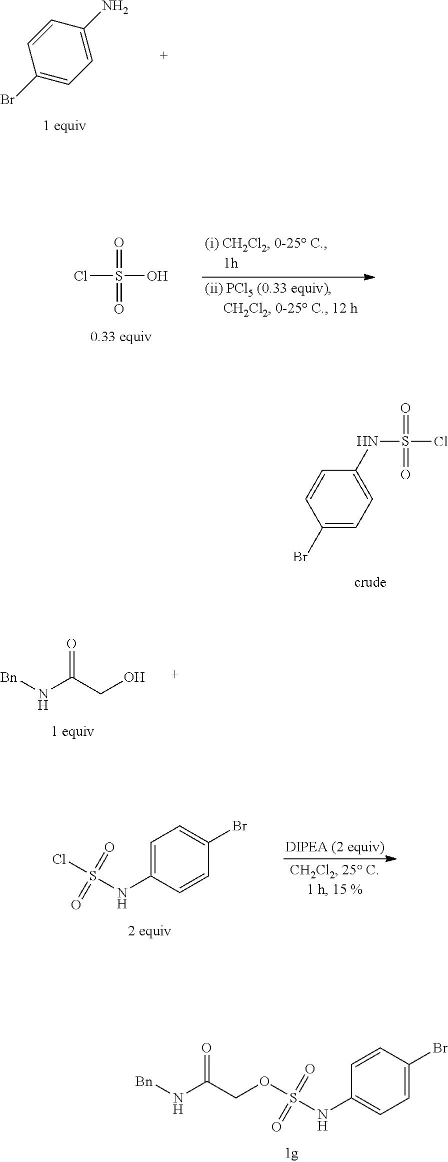 [SMILES: Nc1ccc(Br)cc1 ; O=S(=O)(O)Cl ; O=S(=O)(Cl)Nc1ccc(Br)cc1 ; O=C(CO)NCc1ccccc1 ; O=S(=O)(Cl)Nc1ccc(Br)cc1 ; O=C(COS(=O)(=O)Nc1ccc(Br)cc1)NCc1ccccc1]

To a stirred solution of 4-bromo aniline (172 mg, 1 mmol) in CH₂Cl₂ (1 mL), chloro methane sulfonyl chloride (22 μL, 0.33 mmol) was added at 0° C. The reaction mixture was stirred at room temperature for 1 h. After completion of the reaction organic layer was concentrated in vacuo. The crude product was dissolved CH₂Cl₂ (1 mL) and PCl₅ (68 mg, 0.33 mmol) was added at 0° C. The reaction mixture was stirred at room temperature for 12 h. After completion of the reaction (as monitored by LC-MS), the reaction mixture is filtered and washed with dichloromethane. The filtrate was concentrated and used as such for the next reaction.

To a stirred solution of Pre-1 (16.5 mg, 0.1 mmol) in CH₂Cl₂ (1 mL), 4-bromo phenylsulfamoyl chloride (0.2 mmol), and DIPEA (35 μL, 0.2 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×2 mL). The combined organic layer was concentrated in vacuo and the crude product was purified by preparative HPLC using water:ACN (0.1% formic acid) solvent gradient to afford white solid 1g in 6 mg (15% yield).

¹H NMR (500 MHz, CDCl₃) δ: 4.45 (d, J 5.8 Hz, 2H), 4.66 (s, 2H), 6.36-6.49 (m, 1H), 7.04-7.11 (m, 2H), 7.17 (s, 1H), 7.23 (d, J 6.9 Hz, 2H), 7.32-7.39 (m, 3H), 7.43-7.50 (m, 2H).

¹³C NMR (126 MHz, CDCl₃) δ: 43.7, 68.3, 114.6, 122.8, 128.1, 128.2, 129.2, 133.1, 137.6, 165.8, 173.3.

ESI-MS (m/z): calculated for C₁₅H₁₆Br⁸¹N₂O₄S [M+H]+:401.00; found: [M+H]+:401.22. C₁₅H₁₆Br⁷⁹N₂O₄S [M+H]+: 399.00; found: [M+H]+:399.15.

### Synthesis of 2-(benzylamino)-2-oxoethyl Dimethylsulfamate (1h)

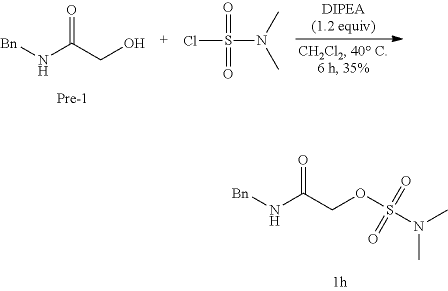

To a stirred solution of Pre-1 (16.5 mg, 0.1 mmol) in CH₂Cl₂ (1 mL), N,N-dimethylsulfamoyl chloride (16.9 mg, 0.12 mmol), and DIPEA (20.4 μL, 0.12 mmol) were added at 40° C. The reaction mixture was stirred at room temperature for 6 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 1h in 9.5 mg (35% yield).

¹H NMR (500 MHz, CD₃OD) δ: 2.92 (s, 6H), 4.46 (s, 2H), 4.63 (s, 2H), 7.23-7.31 (m, 1H), 7.33 (s, 4H).

¹³C NMR (125 MHz, CD₃OD) δ: 38.8, 44.0, 48.6, 68.3, 128.5, 128.8, 129.7, 139.7, and 168.6.

ESI-MS (m/z): calculated for C₁₁H₁₇N₂O₄S [M+H]+: 273.09; found: [M+H]+:273.86.

### Synthesis of N-benzyl-2-(methylsulfonyl)acetamide (1i)

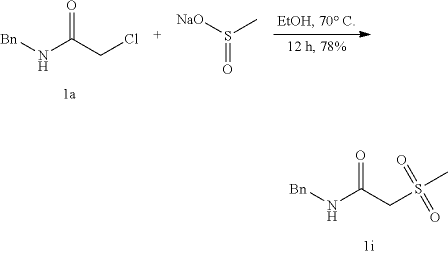

To a stirred solution of 1a (18.3 mg, 0.1 mmol) in ethanol (1 mL), sodium methanesulfinate (20.4 mg, 0.2 mmol) was added at 25° C. The reaction mixture was stirred at room temperature for 12 h at 70° C. After completion of the reaction (as monitored by LC-MS), ethanol was concentrated in vacuo and the crude product was purified by preparative HPLC using water:ACN (0.1% formic acid) solvent gradient to afford white solid 1i in 17.7 mg (78% yield).

ESI-MS (m/z): calculated for C₁₀H₁₄NO₃S [M+H]+: 228.07; found: [M+H]+:228.22.

### Synthesis of N-benzyl-2-(phenylsulfonyl)acetamide (1j)

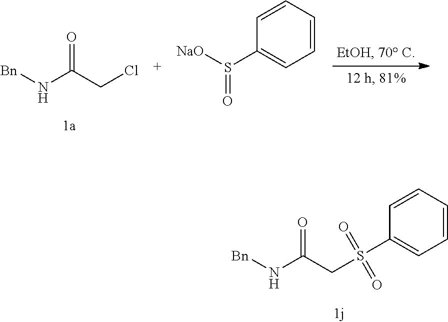

To a stirred solution of 1a (23 mg, 0.1 mmol) in ethanol (1 mL), sodium phenylsulfinate (20.2 mg, 0.2 mmol) was added at 25° C. The reaction mixture was stirred at room temperature for 12 h at 70° C. After completion of the reaction (as monitored by LC-MS), ethanol was concentrated in vacuo and the crude product was purified by preparative HPLC using water:ACN (0.1% formic acid) solvent gradient to afford white solid 1j in 23.4 mg (81% yield).

ESI-MS (m/z): calculated for C₁₅H₁₆NO₃S [M+H]+: 290.09; found: [M+H]+:290.65.

### Synthesis of N-benzyl-2-hydroxyacetamide (Pre-2)

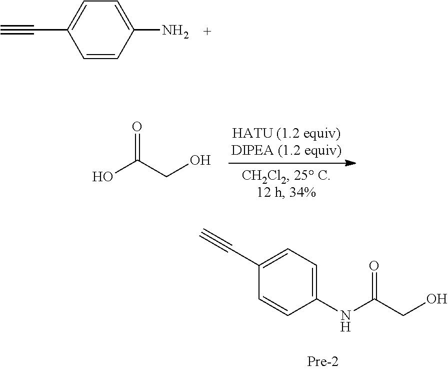

To a stirred solution of 4-ethynylaniline (117 mg, 1 mmol) in anhydrous DCM (6 mL), HATU (456 mg, 1.2 mmol) and DIPEA (200 μL, 1.2 mmol) were added at 25° C. The reaction mixture was stirred at room temperature for 12 h. After completion of the reaction (as monitored by LC-MS), the reaction mixture was evaporated under vacuo and the crude product was purified using combi flash column chromatography with MeOH:EtOAc (2:8) as eluent to give Pre-2 was colorless solid in 59 mg (yield=34%).

¹H NMR (500 MHz, CDCl₃): δ: 3.00 (s, 1H), 3.24 (br. s., 1H), 4.02 (s, 3H), 7.35 (d, J 8.0 Hz, 2H), 7.46 (d, J 8.1 Hz, 2H).

¹³C NMR (126 MHz, CDCl₃) δ: 65.8, 80.8, 87.2, 121.9, 123.4, 136.8, 141.5, 175.3 (s).

ESI-MS (m/z): calculated for C₁₀H₉NO₂S [M+H]+: 176.07; found: [M+H]+:176.09.

### Synthesis of 2-((4-ethynylphenyl)amino)-2-oxoethyl Benzylsulfamate (2a)

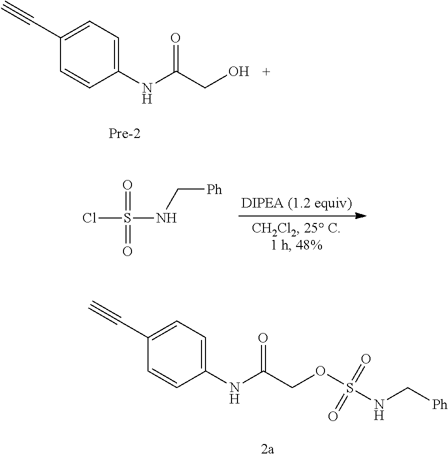 [SMILES: C#Cc1ccc(NC(=O)CO)cc1 ; O=S(=O)(Cl)NCc1ccccc1 ; C#Cc1ccc(NC(=O)COS(=O)(=O)NCc2ccccc2)cc1]

To a stirred solution of Pre-2 (17.5 mg, 0.1 mmol) in CH₂Cl₂ (1 mL), benzylsulfamoyl chloride (24 mg, 0.12 mmol), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 2a in 16.5 mg (48% yield).

¹H NMR (400 MHz, CDCl₃) δ: 3.08 (s, 1H), 4.38 (d, J 5.7 Hz, 2H), 4.54-4.57 (m, 2H), 5.17 (t, J=5.5 Hz, 1H), 7.33-7.41 (m, 4H), 7.42-7.53 (m, 4H), 7.80 (br. s., 1H).

¹³C NMR (100 MHz, CDCl₃) δ: 48.4, 67.6, 83.4, 120.2, 128.4, 129.0, 129.4, 133.3, 135.7, 137.1, 163.7.

ESI-MS (m/z): calculated for C₁₇H₁₇N₂O₄S [M+H]+: 345.09; found: [M+H]+:345.67.

### Synthesis of 2-((4-ethynylphenyl)amino)-2-oxoethyl Phenylsulfamate (2b)

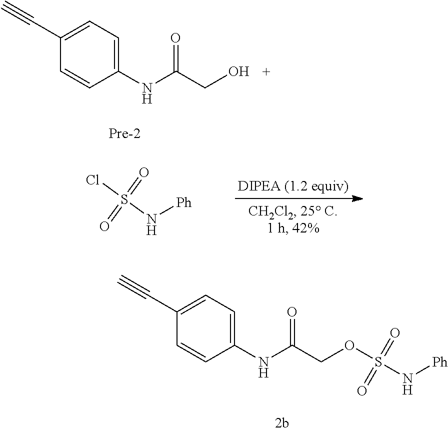 [SMILES: C#Cc1ccc(NC(=O)CO)cc1 ; O=S(=O)(Cl)Nc1ccccc1 ; C#Cc1ccc(NC(=O)COS(=O)(=O)Nc2ccccc2)cc1]

To a stirred solution of Pre-2 (17.5 mg, 0.1 mmol) in CH₂Cl₂ (1 mL), phenylsulfamoyl chloride (22.5 mg, 0.12 mmol), and DIPEA (20.4 μL, 0.12 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 2b in 13.8 mg (42% yield).

¹H NMR (500 MHz, CDCl₃) δ: 3.07 (s, 1H), 4.74 (s, 2H), 7.26 (br. s., 2H), 7.28 (br. s., 2H), 7.37-7.42 (m, 3H), 7.44-7.47 (m, 2H).

¹³C NMR (126 MHz, CDCl₃) δ: 67.9, 68.6, 83.0, 119.8, 121.2, 126.5, 129.9, 133.0, 137.3, 141.0, 163.2.

ESI-MS (m/z): calculated for C₁₆H₁₅N₂O₄S [M+H]+: 331.08; found: [M+H]+:331.97.

### Synthesis of 2-chloro-N-(4-ethynylphenyl)acetamide (2c)

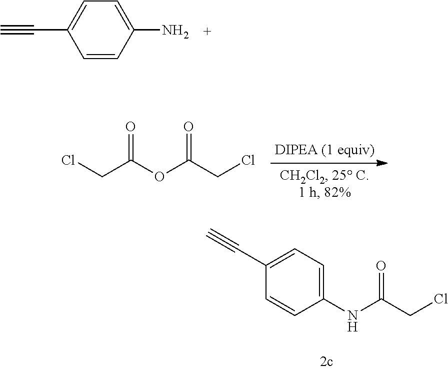

To a stirred solution of 4-ethynylaniline (11.7 mg, 1 mmol) in anhydrous DCM (0.5 mL), DIPEA (17.8 μL, 1 mmol) and chloroacetic anhydride (17 mg, 1 mmol) were added at 25° C. The reaction mixture was stirred at room temperature for 1 h. After completion of the reaction (as monitored by LC-MS), CH₂Cl₂ was concentrated in vacuo and the crude compound was purified by preparative HPLC using water:ACN (0.100 formic acid) solvent gradient to afford white solid 2c in 15.8 mg (yield=820%).

¹H NMR (400 MHz, CDCl₃) δ: 3.08 (s, 1H), 4.20 (s, 2H), 7.41-7.60 (m, 4H), 8.28 (br. s., 1H).

¹³C NMR (100 MHz, CDCl₃) δ: 42.8, 83.0, 118.8, 119.6, 133.0, 137.0, 163.8.

ESI-MS (m/z): calculated for C₁₀H₉ClNO [M+H]+: 194.04; found: [M+H]+: 194.82.

### Example 2

### Reactivity of Sulfamate Acetamide Compounds

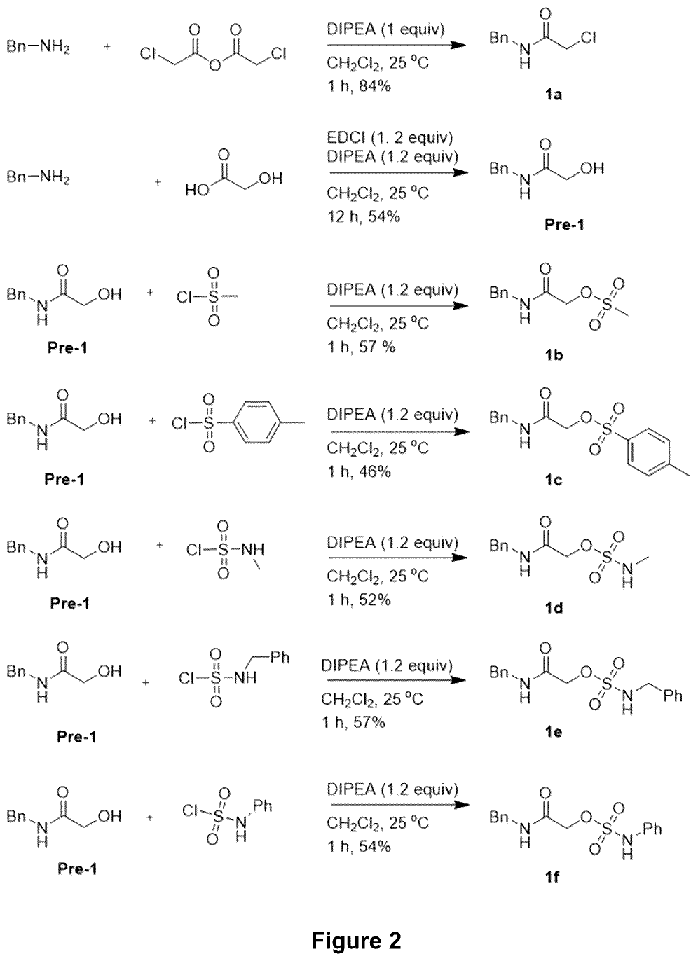

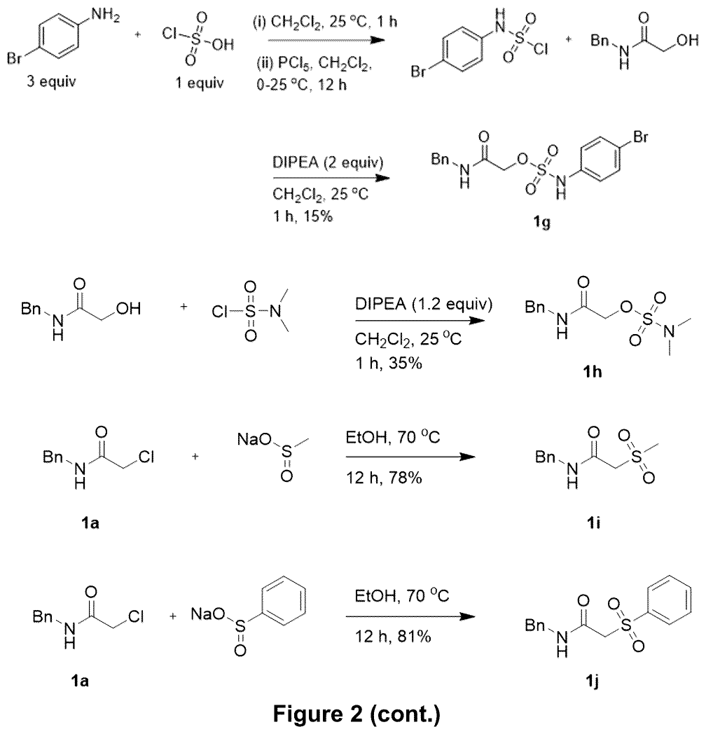

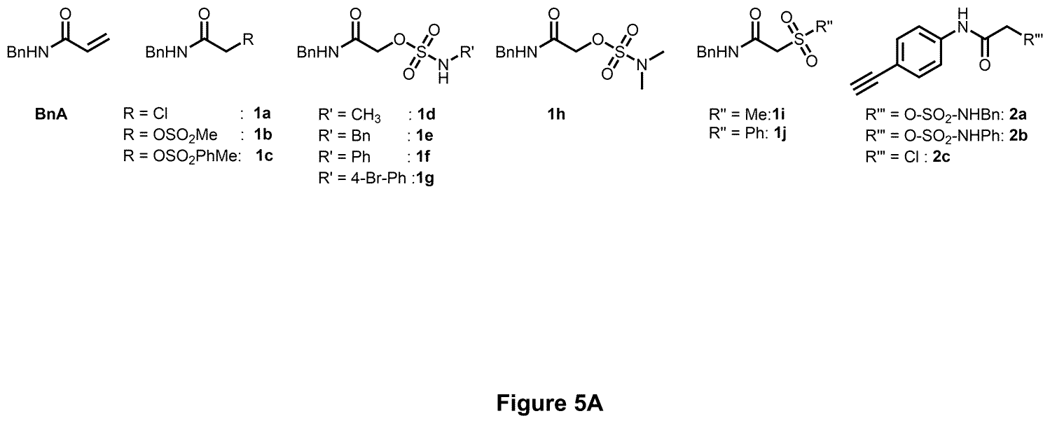

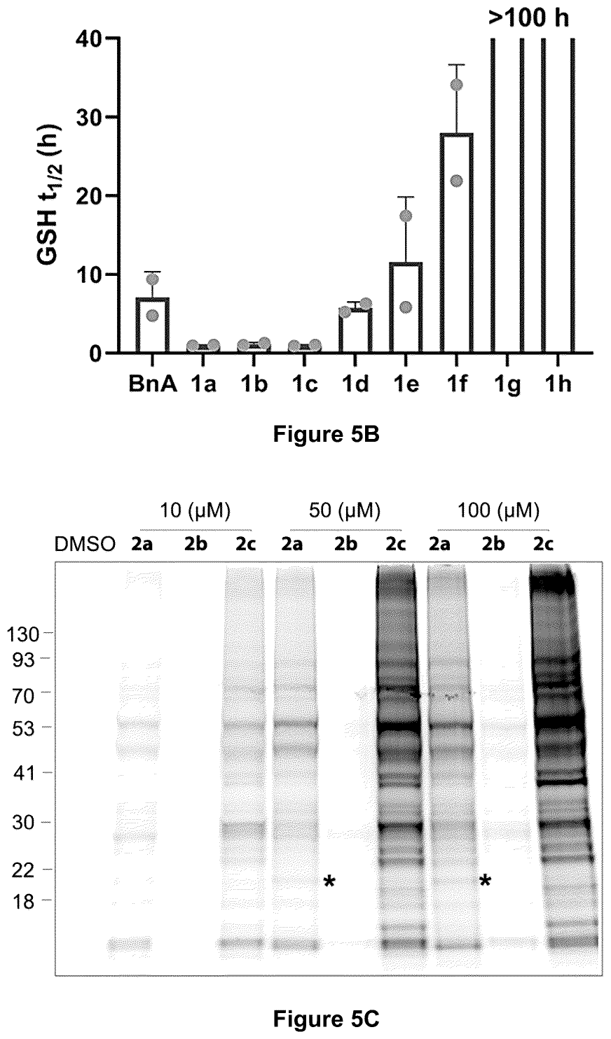

To investigate the reactivity of sulfamate acetamide compounds, nine sulfo-based model electrophile compounds were synthesized (See Example 3) including two sulfonates (1b and 1c) five sulfamates (1d-1h), and two sulfones (1i and 1j; FIGS. 2 and 5A)

The model compounds are as shown in the Table 3.

TABLE 3
Model compounds.

| Compound | |
|---|---|
| number | Structure |
| 1a | 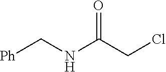 [SMILES: O=C(CCl)NCc1ccccc1] |
| 1b | 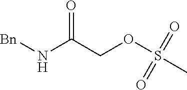 [SMILES: CS(=O)(=O)OCC(=O)NCc1ccccc1] |
| 1c | 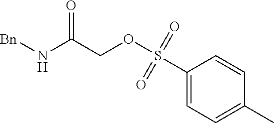 [SMILES: Cc1ccc(S(=O)(=O)OCC(=O)NCc2ccccc2)cc1] |
| 1d | 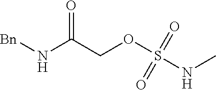 [SMILES: CNS(=O)(=O)OCC(=O)NCc1ccccc1] |
| 1e | 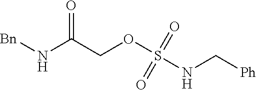 [SMILES: O=C(COS(=O)(=O)NCc1ccccc1)NCc1ccccc1] |
| 1f | 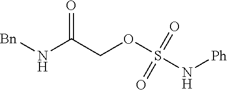 [SMILES: O=C(COS(=O)(=O)Nc1ccccc1)NCc1ccccc1] |
| 1g | 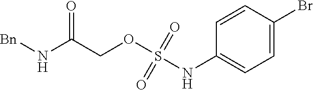 [SMILES: O=C(COS(=O)(=O)Nc1ccc(Br)cc1)NCc1ccccc1] |
| 1h | 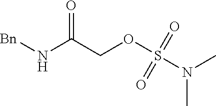 [SMILES: CN(C)S(=O)(=O)OCC(=O)NCc1ccccc1] |
| 1i | 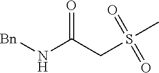 |
| 1j | 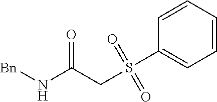 [SMILES: O=C(CS(=O)(=O)c1ccccc1)NCc1ccccc1] |
| 2a | 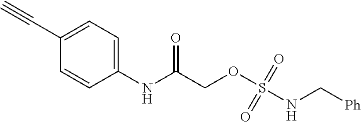 [SMILES: C#Cc1ccc(NC(=O)COS(=O)(=O)NCc2ccccc2)cc1] |
| 2b | 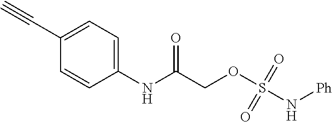 [SMILES: C#Cc1ccc(NC(=O)COS(=O)(=O)Nc2ccccc2)cc1] |
| 2c | 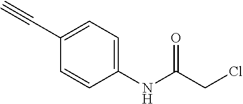 |

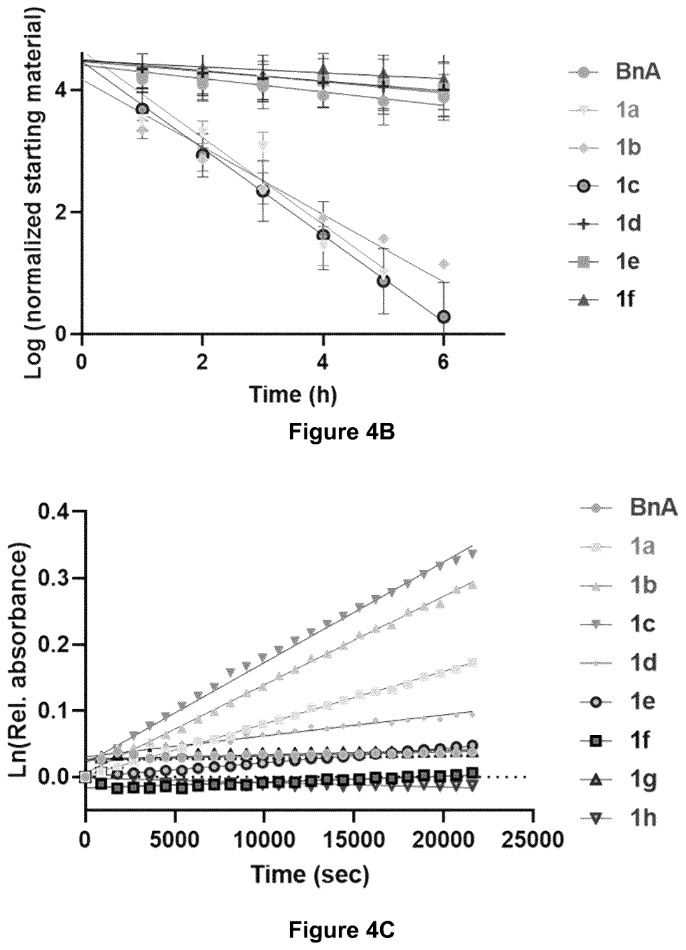

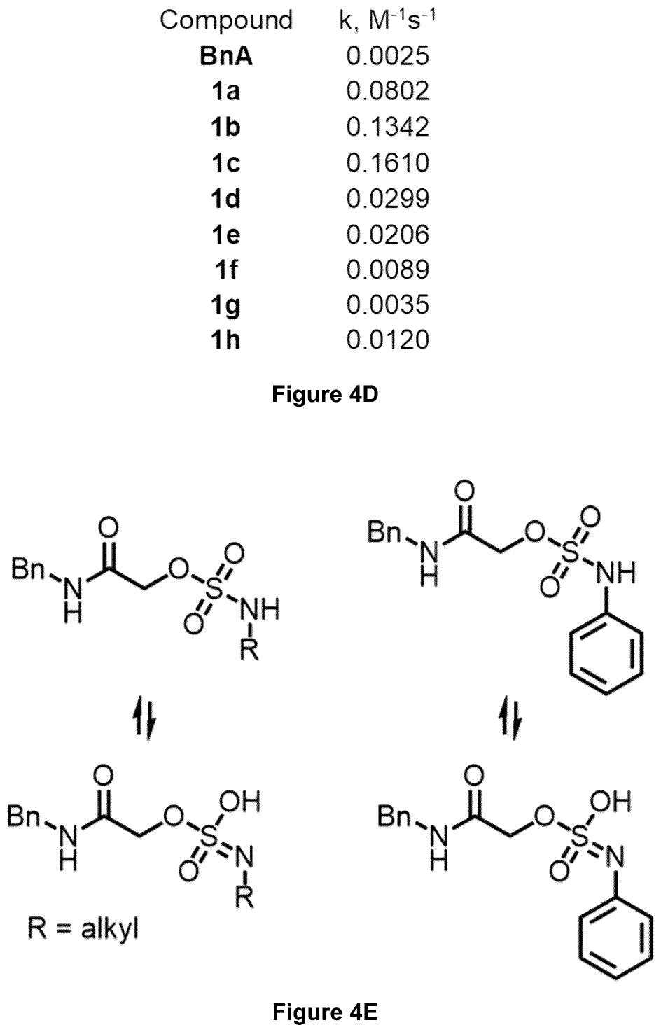

GSH consumption assays were conducted (5 mM GSH, 200 μM electrophile at pH 8, 14° C.; 4-nitro cyano-benzene was used as an internal standard) for all the sulfo compounds as well as benzyl acrylamide (BnA) and chloroacetamide (1a) (FIG. 5A). A sample from the reaction mixture was injected to an LC/MS every hour and the decrease in starting material was quantified over the course of the reaction. For example, the LC/MS chromatogram (at 220 nm) of sulfamate 1d at t=0 h and at t=5 h shows an increase in GSH adduct and a decrease in starting material (FIG. 4A). The sulfonate esters, mesyl (1b) and tosyl (1c) groups showed similar reactivities to the chloroacetamide (1a) with a half-life of 50 min. On the other hand, methyl sulfamate (1d) and benzyl sulfamate (1e) showed an order of magnitude less reactivity than chloroacetamide (1a) with equal or lower reactivity to that of the unsubstituted acrylamide (BnA; FIG. 4B).

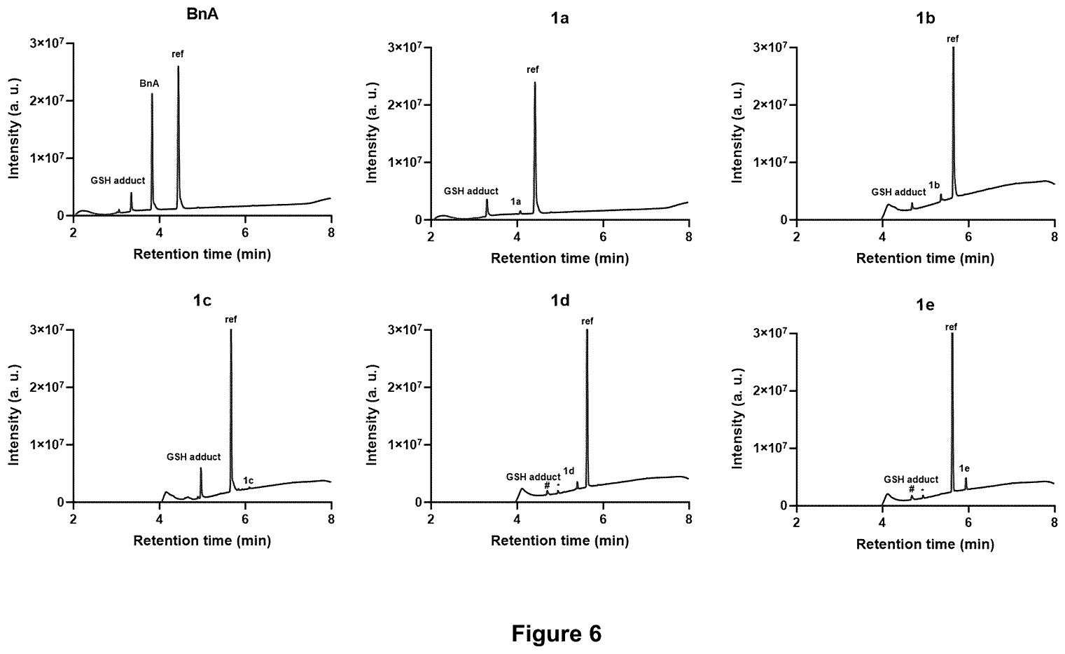

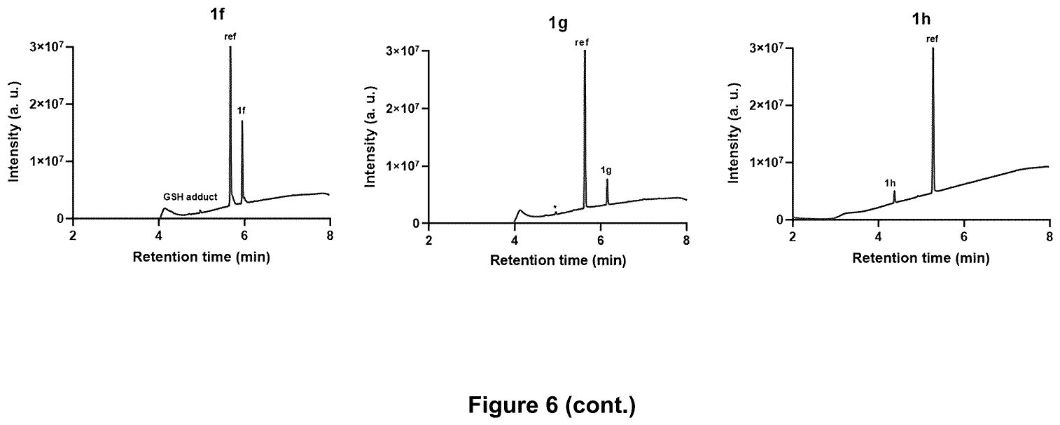

Surprisingly, phenyl sulfamate (1f; t1/2=˜25 h) and 4-bromo phenyl sulfamate (1g; t1/2>100 h) exhibit much lower reactivity, possibly because the release of electrons from the amine to the sulfur increases conjugation and stabilization (FIG. 4E). Finally, dimethyl sulfamate (1h) also did not react under these reaction conditions (t1/2>100 h). α-sulfone acetamides (1i and 1j) did not form a covalent bond with GSH under the assay conditions or even at temperatures up to 37° C. for 24 h (FIG. 6). The thiol reactivities of these model compounds were assessed using a DTNB reactivity assay (FIG. 4C), which showed similar results to the GSH consumption assay (FIG. 5B; FIG. 4D).

Sulfamate model compounds (1d-1j) are an order of magnitude less reactive to GSH than the corresponding chloroacetamide (1a) and sulfonate compounds (1b-1c). Whereas the aryl-substituted and secondary amine substituted sulfamates show even drastically lower reactivity (FIG. 5B). This may be because the electronics of the amine reduces the electrophilicity of the α-carbon of the sulfamate acetamides (FIG. 4E). The proteomic reactivity of the sulfamates reflected similar reactivity trends with the more reactive chloroacetamide (2c) and benzyl sulfamate acetamide (2a) enriching more proteins than the low reactivity phenyl sulfamate acetamide (2b; FIG. 5).

The fact that additional bands were detected with the phenyl sulfamate acetamide compared to the chloroacetamide may suggest that the extra recognition of the phenyl sulfamate mediates additional interactions with some protein targets. Similar to substituted methacrylamides, these compounds also leave identical adducts on proteomically labeled cysteines. Hence, mixtures of sulfamates can serve as probes for quantitative chemoproteomics with potentially increased coverage.

### Example 3

### Synthesis of Sulfamate Compounds (FIG. 9)

### Synthesis of N-(1,1-dioxidotetrahydrothiophen-3-VO-2-hydroxy-N-neopentylacetamide (Pre-4)

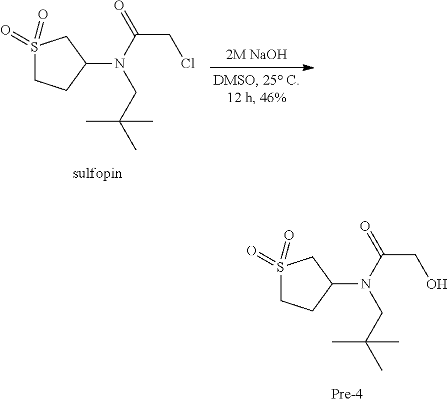 [SMILES: CC(C)(C)CN(C(=O)CCl)C1CCS(=O)(=O)C1 ; CC(C)(C)CN(C(=O)CO)C1CCS(=O)(=O)C1]

To a stirred solution of sulfopin (281 mg, 1 mmol) in DMSO (2 mL), NaOH (5 M, 2 mL) was added at 25° C. The reaction mixture was stirred at room temperature for 12 h. After completion of the reaction (as monitored by LC-MS), water (1 mL) was added. The aqueous layer was extracted with Ethyl acetate (3×3 mL). The combined organic layer was concentrated in vacuo and the crude product was purified by preparative HPLC using water:ACN (0.1% formic acid) solvent gradient to afford white solid Pre-4 in 121 mg (46% yield).

¹H NMR (500 MHz, CDCl3) δ: 1.01 (s, 10H), 2.49-2.64 (m, 2H), 2.92-2.99 (m, 1H), 3.02 (s, 1H), 3.05-3.10 (m, 1H), 3.16 (dd, J=12.6, 8.3 Hz, 2H), 3.71 (dt, J 12.9, 9.2 Hz, 1H), 3.79 (dd, J 12.6, 9.8 Hz, 1H), 3.90-3.99 (m, 1H), 4.11-4.22 (m, 2H).

¹³C NMR (126 MHz, CDCl3) δ: 26.6, 28.1, 33.6, 49.5, 50.5, 57.2, 60.1, 60.6, 173.2.

ESI-MS (m/z): calculated for C₁₁H₂₂NO₄S [M+H]+: 264.12; found: [M+H]+:264.16.

### Synthesis of 2-((1,1-dioxidotetrahydrothiophen-3-yl)(neopentyl)amino)-2-oxoethyl Methanesulfonate (4a)

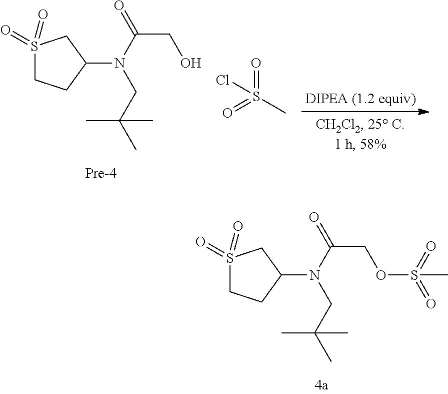

To a stirred solution of Pre-4 (13 mg, 0.05 mmol) in CH₂Cl₂ (1 mL), methane sulfonyl chloride (4.6 μL, 0.06 mmol, d=1.48), and DIPEA (10.2 μL, 0.06 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 4a in 9.8 mg (74% yield).

¹H NMR (500 MHz, CDCl3): δ: 1.03 (s, 9H), 2.50-2.59 (m, 2H), 3.01 (s, 1H), 3.04-3.19 (m, 3H), 3.27 (s, 3H), 3.64-3.83 (m, 2H), 3.93 (d, J 8.0 Hz, 1H), 4.86 (s, 2H).

HR-MS (m/z): calculated for C₁₂H₂₄NO₆S₂ [M+Na]+: 342.1045; found: [M+Na]+:342.1045.

### Synthesis of 2-((1,1-dioxidotetrahydrothiophen-3-yl)(neopentyl)amino)-2-oxoethyl Methylsulfamate (4b)

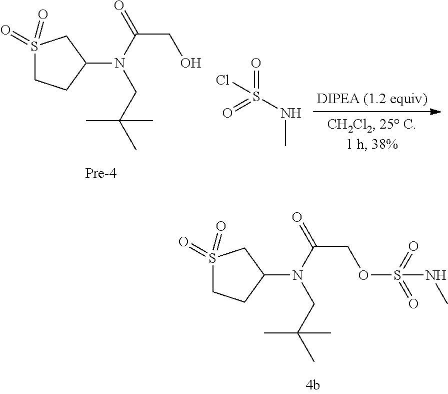

To a stirred solution of pre-4 (13 mg, 0.05 mmol) in CH₂C₁₂ (1 mL), N-methylsulfamoyl chloride (7.74 mg, 0.06 mmol), and DIPEA (10.2 μL, 0.06 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 4b in 6.74 mg (38% yield).

¹H NMR (500 MHz, CDCl₃) δ: 1.03 (s, 9H), 2.55 (d, J 8.5 Hz, 2H), 2.91 (s, 3H), 2.97-3.09 (m, 2H), 3.09-3.20 (m, 2H), 3.63-3.71 (m, 1H), 3.73-3.82 (m, 1H), 3.85-3.98 (m, 1H), 4.80 (s, 2H), 5.21 (br. s., 1H).

¹³C NMR (125 MHz, CDCl₃) δ: 26.6, 28.0, 30.4, 33.7, 49.3, 50.5, 57.7, 61.3, 67.5, 168.0.

HR-MS (m/z): calculated for C₁₂H₂₄N₂NaO₆S₂ [M+Na]+: 379.0973; found: [M+Na]+:379.0964.

### Synthesis of 2-((1,1-dioxidotetrahydrothiophen-3-yl)(neopentyl)amino)-2-oxoethyl Benzylsulfamate (4c)

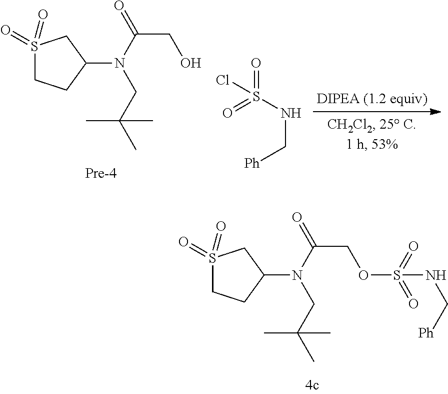

To a stirred solution of Pre-4 (13 mg, 0.05 mmol) in CH₂Cl₂ (1 mL), benzylsulfamoyl chloride (12.3 mg, 0.06 mmol), and DIPEA (10.2 μL, 0.06 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×1 mL). The combined organic layer was concentrated in vacuo and the crude product was purified by preparative HPLC using water:ACN (0.1% formic acid) solvent gradient to afford white solid 4c in 11.4 mg (53% yield).

¹H NMR (500 MHz, CDCl₃) δ: 1.00 (s, 9H), 2.50 (m, 2H), 3.00 (m, 2H), 3.11 (m, 2H), 3.60 (m, 1H), 3.71 (m, 1H), 3.89 (quin, J 7.9 Hz, 1H), 4.39 (d, J 5.4 Hz, 2H), 4.76 (s, 2H), 5.64 (br. s., 1H), 7.35 (m, 5H).

¹³C NMR (125 MHz, CDCl₃) δ: 26.6, 28.0, 33.7, 48.2, 49.3, 50.4, 57.6, 61.2, 67.4, 128.1, 128.2, 128.8, 136.1, 167.7.

HR-MS (m/z): calculated for C₁₈H₂₈N₂NaO₆S₂[M+Na]+: 455.1286; found: [M+Na]+:455.1279.

### Synthesis of 2-((1,1-dioxidotetrahydrothiophen-3-yl)(neopentyl)amino)-2-oxoethyl Phenylsulfamate (4d)

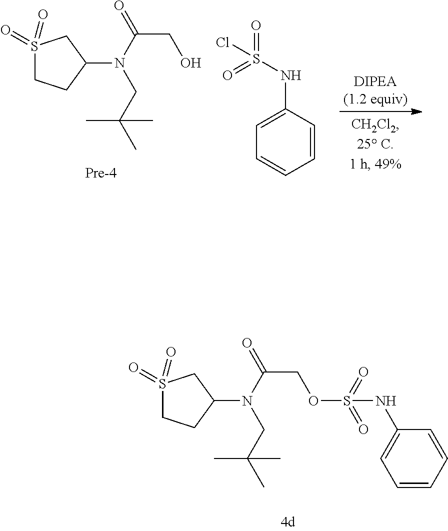

To a stirred solution of pre-4 (13 mg, 0.05 mmol) in CH₂Cl₂ (1 mL), phenylsulfamoyl chloride (11.5 mg, 0.06 mmol), and DIPEA (10.2 μL, 0.06 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×2 mL). The combined organic layer was concentrated in vacuo and the crude product was purified by preparative HPLC using water:ACN (0.1% formic acid) solvent gradient to afford white solid 4d in 10.2 mg (49% yield).

¹H NMR (500 MHz, CD3OD) δ: 0.98 (s, 10H), 2.45-2.52 (m, 2H), 2.57 (s, 1H), 3.05 (dt, J 13.0, 6.4 Hz, 1H), 3.11-3.26 (m, 3H), 3.51-3.63 (m, 2H), 4.02 (quin, J 8.0 Hz, 1H), 4.80 (s, 2H), 7.16 (s, 1H), 7.25 (d, J 8.0 Hz, 2H), 7.35 (t, J 7.8 Hz, 2H).

¹³C NMR (126 MHz, CD3OD) δ: 27.8, 28.4, 34.5, 50.7, 51.9, 59.2, 61.7, 68.4, 121.1, 125.7, 130.5, 138.7, 145.7, 168.9.

HR-MS (m/z): calculated for C₁₇H₂₆N₂NaO₆S₂[M+Na]+: 441.1130; found: [M+Na]+:441.1120.

### Synthesis of 2-((1,1-dioxidotetrahydrothiophen-3-yl)(neopentyl)amino)-2-oxoethyl (4-bromophenyl)sulfamate (4e)

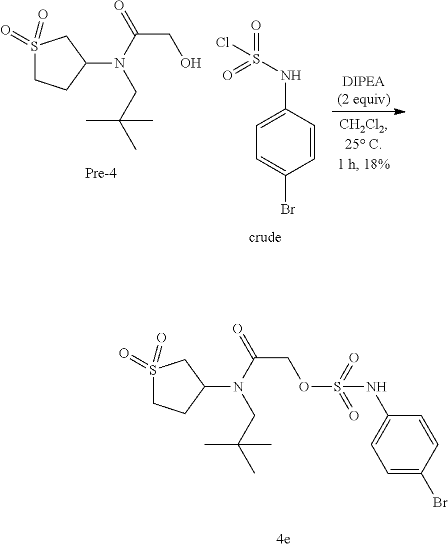

To a stirred solution of Pre-4 (13 mg, 0.05 mmol) in CH₂Cl₂ (1 mL), 4-bromo phenylsulfamoyl chloride (see synthesis of 1g for preparation) (27 mg, 0.1 mmol), and DIPEA (17.6 μL, 0.1 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×2 mL). The combined organic layer was concentrated in vacuo and the crude product was purified by preparative HPLC using water:ACN (0.1% formic acid) solvent gradient to afford white solid 4d in 4.4 mg (18% yield).

¹H NMR (500 MHz, CDCl3) δ: 1.02 (s, 9H), 2.50-2.63 (m, 2H), 2.98-3.05 (m, 1H), 3.05-3.13 (m, 2H), 3.13-3.21 (m, 1H), 3.62-3.71 (m, 1H), 3.73-3.81 (m, 1H), 3.87-3.98 (m, 1H), 4.89 (s, 2H), 7.22 (d, J 8.8 Hz, 2H), 7.46-7.54 (m, 3H).

¹³C NMR (125 MHz, CDCl3) δ: 26.6, 28.1, 33.8, 49.5, 50.6, 57.9, 61.4, 68.8, 124.3, 132.5. quaternary carbons were not detected.

HR-MS (m/z): calculated for C₁₇H₂₅Br⁷⁹N₂NaO₆S₂[M+Na]+: 519.0235; found: [M+Na]+:519.0233, calculated for C₁₇H₂₅Br⁸¹N₂NaO₆S₂[M+Na]+: 521.0215; found: [M+Na]+:521.0214.

### Synthesis of 2-((1,1-dioxidotetrahydrothiophen-3-yl)(neopentyl)amino)-2-oxoethyl p-tolylsulfamate (4f)

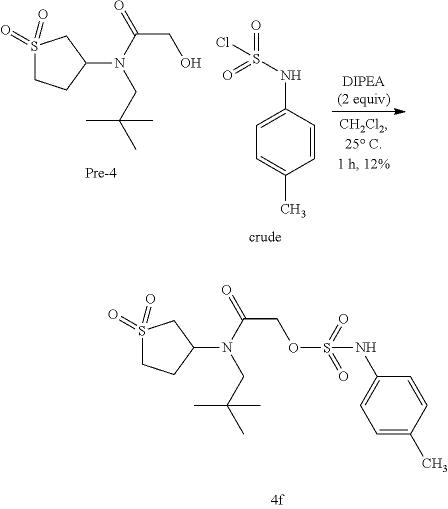

To a stirred solution of pre-4 (13 mg, 0.05 mmol) in CH₂Cl₂ (1 mL), 4-methyl phenylsulfamoyl chloride (see synthesis of 1g for preparation) (11.5 mg, 0.06 mmol), and DIPEA (10.2 μL, 0.06 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×2 mL). The combined organic layer was concentrated in vacuo and the crude product was purified by preparative HPLC using water:ACN (0.1% formic acid) solvent gradient to afford white solid 4d in 2.5 mg (12% yield).

¹H NMR (500 MHz, CD₃OD) δ: 0.95 (s, 10H), 1.26-1.38 (m, 2H), 2.29 (s, 3H), 2.42-2.51 (m, 2H), 3.03 (dt, J 12.9, 6.5 Hz, 1H), 3.15-3.21 (m, 2H), 3.56 (dd, J 13.4, 8.9 Hz, 2H), 3.95-4.05 (m, 1H), 4.71-4.80 (m, 2H), 7.08-7.21 (m, 5H).

¹³C NMR (126 MHz, CD₃OD) δ: 21.4, 28.2, 28.9, 32.3, 51.2, 52.4, 59.7, 62.2, 68.9, 122.3, 131.5, 168.5.

HR-MS (m/z): calculated for C₁₈H₂₉N₂O₆S₂[M+H]+: 433.1467; found: [M+H]+:433.1467.

### Synthesis of 2-((cyclohexylmethyl)(1,1-dioxidotetrahydrothiophen-3-yl)amino)-2-oxoethyl Phenylsulfamate (4g)

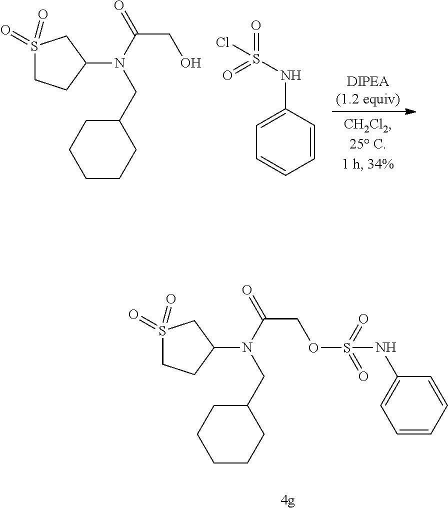

To a stirred solution of alcohol (14.5 mg, 0.05 mmol) in CH₂C₁₂ (1 mL), phenylsulfamoyl chloride (11.5 mg, 0.06 mmol), and DIPEA (10.2 μL, 0.06 mmol) were added at 25° C. The reaction mixture was stirred at room temperature for 1 h. After completion of the reaction (as monitored by LC-MS), water (1 mL) was added. The aqueous layer was extracted with CH₂Cl₂ (3×2 mL). The combined organic layer was concentrated in vacuo and the crude product was purified by preparative HPLC using water:ACN (0.1% formic acid) solvent gradient to afford white solid 4g in 7.5 mg (34% yield).

¹H NMR (500 MHz, CDCl₃) δ: 0.87 (d, J 14.3 Hz, 3H), 0.91-0.98 (m, 2H), 1.13-1.33 (m, 6H), 1.54 (br. s., 2H), 2.41-2.65 (m, 2H), 2.99-3.10 (m, 3H), 3.12 (s, 1H), 3.16 (br. s., 1H), 3.58 (d, J=8.1 Hz, 1H), 3.64-3.76 (m, 1H), 4.00-4.11 (m, 1H), 4.86 (s, 2H), 7.26-7.46 (m, 5H).

¹³C NMR (126 MHz, CDCl₃) δ: 25.7, 26.0, 26.2, 30.7, 30.8, 37.8, 49.9, 50.7, 55.5, 55.9, 68.6, 122.4, 126.1, 129.4, 144.7.

HR-MS (m/z): calculated for C₁₉H₂₈N₂NaO₆S₂[M+Na]+: 467.1286; found: [M+Na]+:467.1286.

### Example 4

### Selectivity of Sulfamate Acetamide Compounds Towards Cysteine

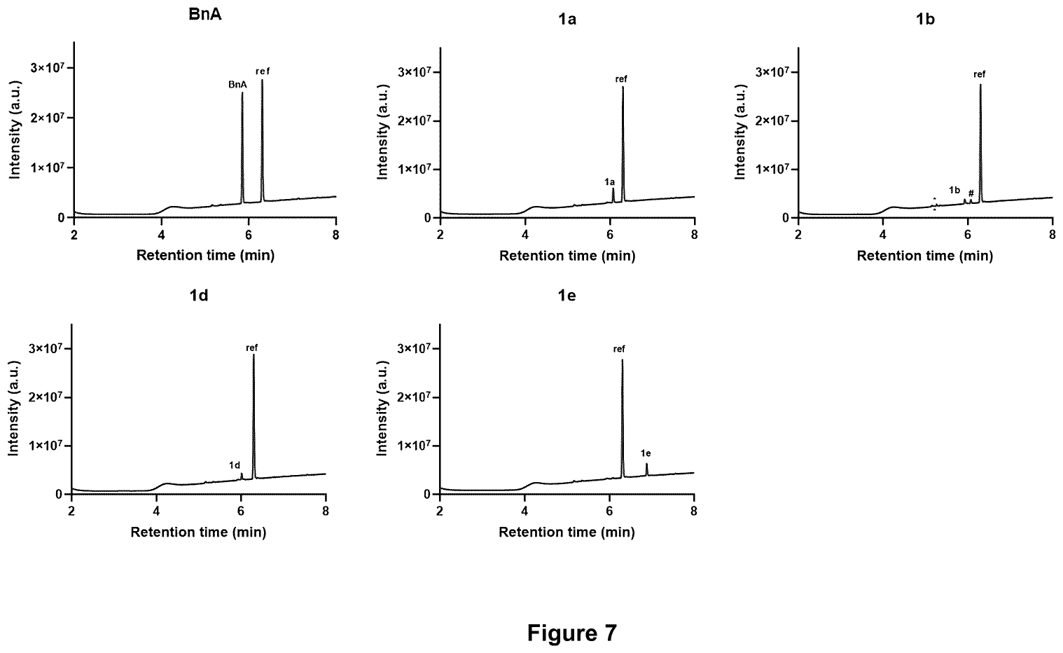

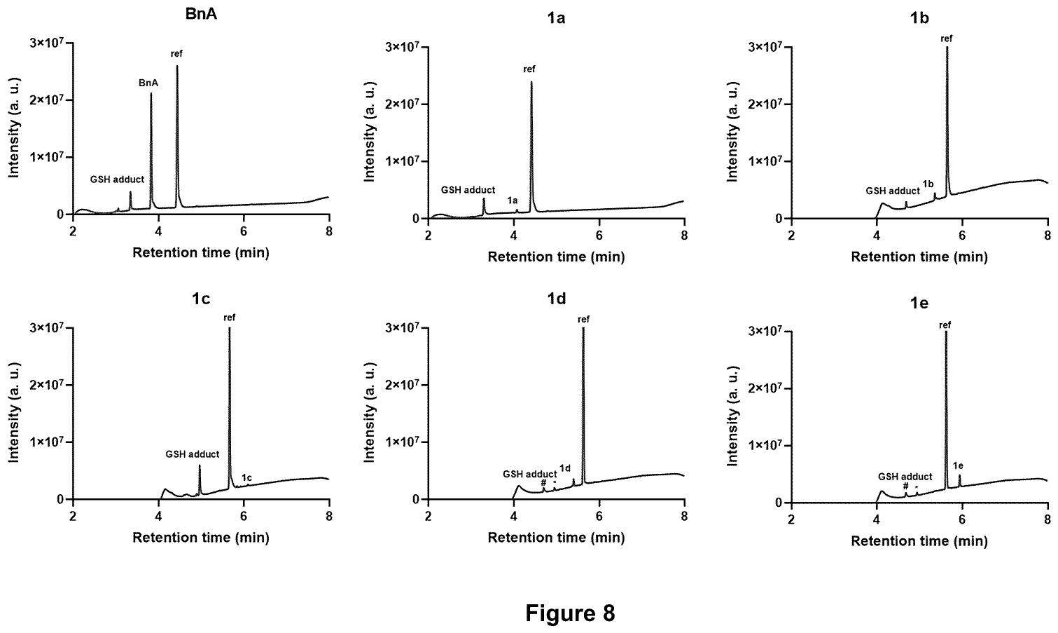

To understand the selectivity of these electrophiles towards cysteine over other nucleophilic amino acids, the glutamic acid and lysine with five model compounds (BnA, 1a, 1b, 1d & 1e) were reacted under stringent reaction conditions (pH 8, 37° C.; 4 days; FIG. 7). Under these conditions, glutamic acid did not react with any of these compounds whereas lysine reacted with BnA, 1a, and 1b (forming 15%, 12%, and 9% respectively of the corresponding lysine adduct). All three sulfamates formed <5% product suggesting that these compounds are more selective towards cysteine than lysine (FIG. 7). Moreover, these sulfamate acetamides showed high buffer stability and did not undergo hydrolysis (<5%) after two days whereas chloroacetamide 1a hydrolyzed (12%) as well as sulfonate acetamides 1b and 1c (26% and 50% respectively; FIG. 8)

### Example 5

### Proteomic Reactivity of Sulfamate Acetamide Compounds

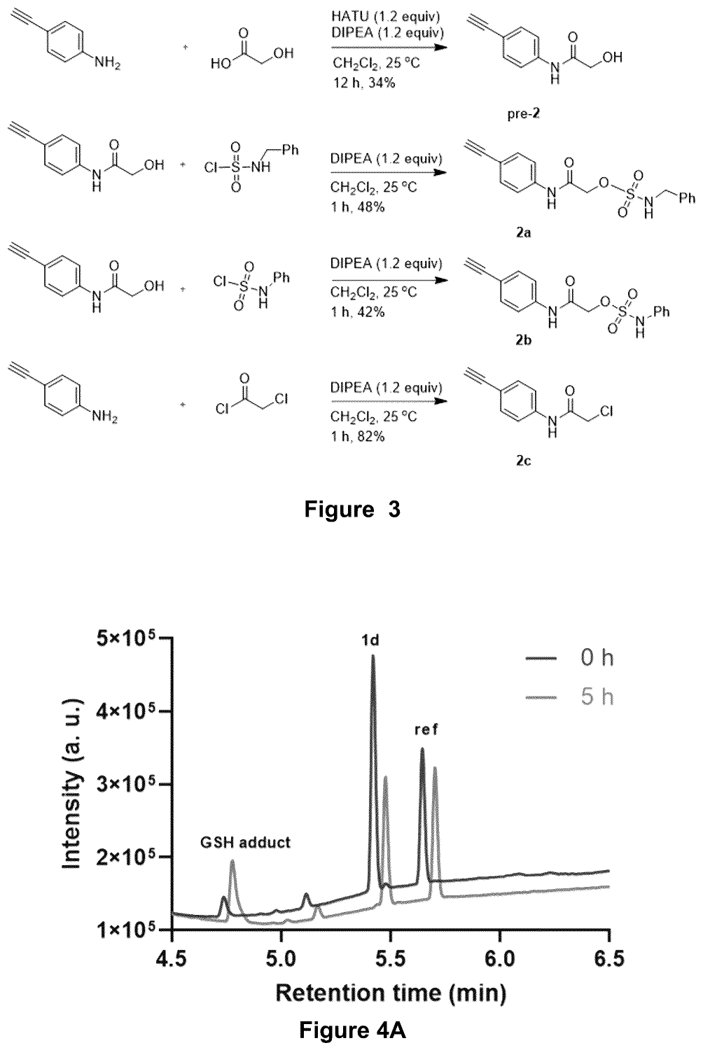

Sulfamate acetamides show attenuated and tunable proteomic reactivity. To assess proteomic selectivity in cells, two sulfamate acetamides (2a and 2b) and a chloroacetamide (2c) with an alkyne functionality have been synthesized (FIG. 3 and FIG. 4A). Mino cells were treated for two hours with either DMSO or 2a-c. Then, the cells were lysed, labeled the alkynes via copper-catalyzed “click chemistry” (CuAAC) with TAMRA-azide, and imaged the adducts via in-gel fluorescence (FIG. 4C). Similar to their reactivity pattern in the GSH reactivity assay, these compounds labeled various amounts of proteins under cellular conditions. The most reactive chloroacetamide labeled the highest number of proteins followed by benzyl sulfamate acetamide and phenyl sulfamate acetamide. Interestingly, there is at least one example in which sulfamate 2a labeled a distinct protein that was not labeled by the chloroacetamide, potentially due to having extra recognition mediated through the sulfamate.

### Example 6

### Sulfamate Compounds as Potent Inhibitors of Pin1

Sulfopin (FIG. 1) was developed as a selective covalent inhibitor of Pin1 which blocks Mycdriven tumors in vivo [Dubiella, C. et al. Nat. Chem. Biol. 17, 954-963 (2021). which is hereby incorporated by reference in its entirety.]. Sulfopin has a chloroacetamide electrophile, and previous attempts to switch it to an acrylamide, or alternative warheads did not result in efficient covalent binders (data not shown). sulfamate and sulfonate compounds were synthesized including one sulfonate (4a), two alkyl sulfamates (4b, 4c), and four aryl sulfamate (4d-4g) as potential Pin1 inhibitors (FIG. 9).

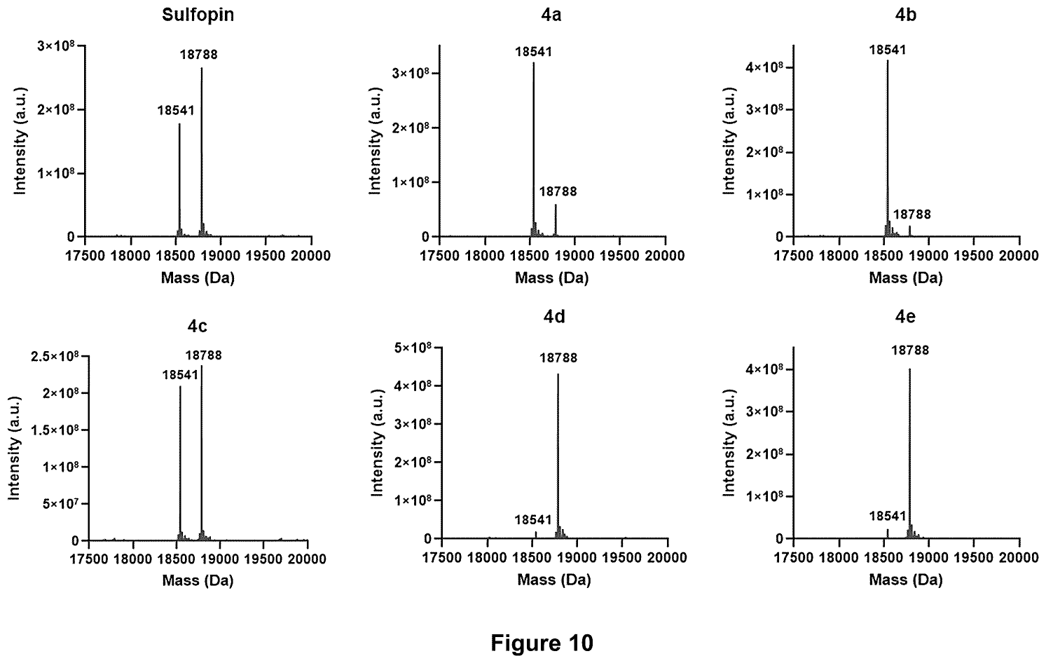

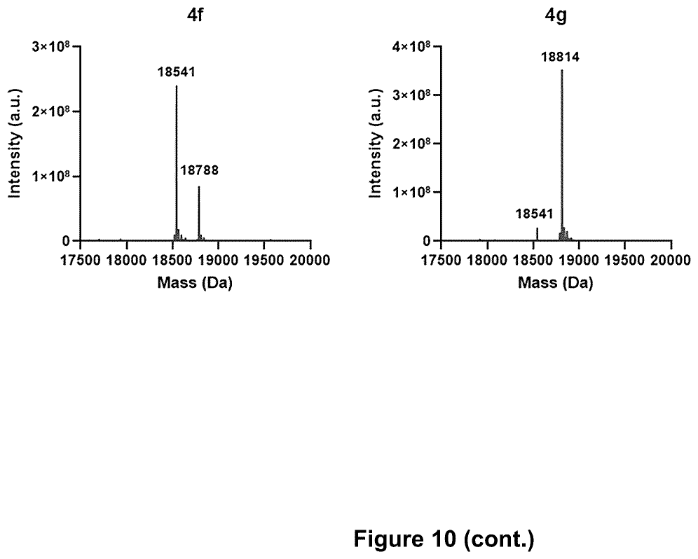

Initially, these compounds (4a-4g; 2 μM) were incubated with Pin1 (2 μM; 50 mM Tris buffer; pH 7.5, 1 h; 25° C.) to check covalent labeling by intact protein MS (FIG. 11B,C; FIG. 10).

Under these conditions Sulfopin labeled 59% as previously reported [Dubiella, C. et al. Nat. Chem. Biol. 17, 954-963 (2021) which is hereby incorporated by reference in its entirety], the sulfonate (4a) labeled 18% whereas alkyl sulfamates (4b, 4c) labeled 16% and 56% respectively. Phenyl sulfamate (4d) and 4-bromo phenyl sulfamate (4e) showed >90% labeling. 4g contains both a cyclohexyl group instead of the tertbutyl as well as a phenyl sulfamate electrophile, and was able to label >95% of Pin1 under these conditions.

To characterized the kinetics of Pin1 binding, by incubating Pin1 (0.5 μM) with various concentrations of sulfopin or 4d (100 mM Tris buffer; pH 7.4; 14° C.) and monitoring the reaction by LC-MS at different time points, and determined K_{I} and K_{inact} for both compounds. Although the K_{inact} values for both sulfopin (2.31 s⁻¹) and 4d (2.48 s⁻¹) are similar, the K₁ value of 4d (43.6 μM) is better than for sulfopin (216.9 μM; FIG. 17), perhaps suggesting that the sulfamate group mediates additional non-covalent interactions with the protein.

To assess the reactivity of these Sulfopin-based derivatives—sulfamate compounds, a DTNB thiol reactivity assay (FIG. 12) was carried out and found that compounds 4c, 4d, and 4e show lower thiol reactivity than Sulfopin whereas 4g showed slightly higher reactivity (FIG. 11C).

To understand the binding mode of these compounds, the pre-reacted compounds were modeled in complex with Pin1 (FIG. 15). The modeling suggests that the sulfamate group has the potential to form additional hydrogen bonds with the protein and the sulfamate side-chain has room to propagate into an additional pocket on the protein that may mediate extra recognition which can explain the increased labeling despite lower thiol reactivity. Next, the buffer stability of the Sulfamate acetamides in PBS buffer (pH 8; 37° C.) was examined.

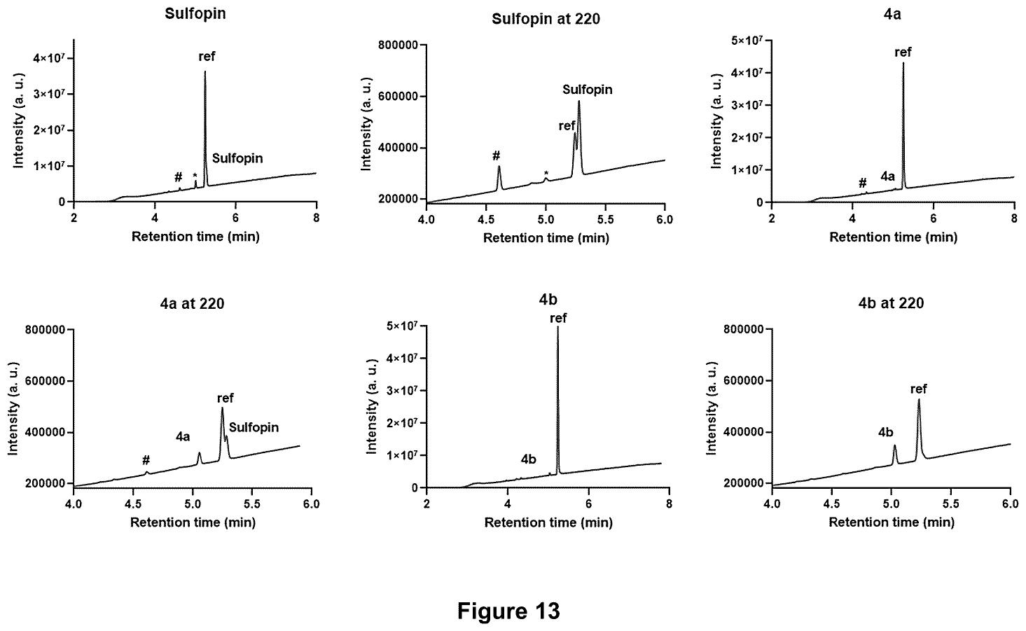

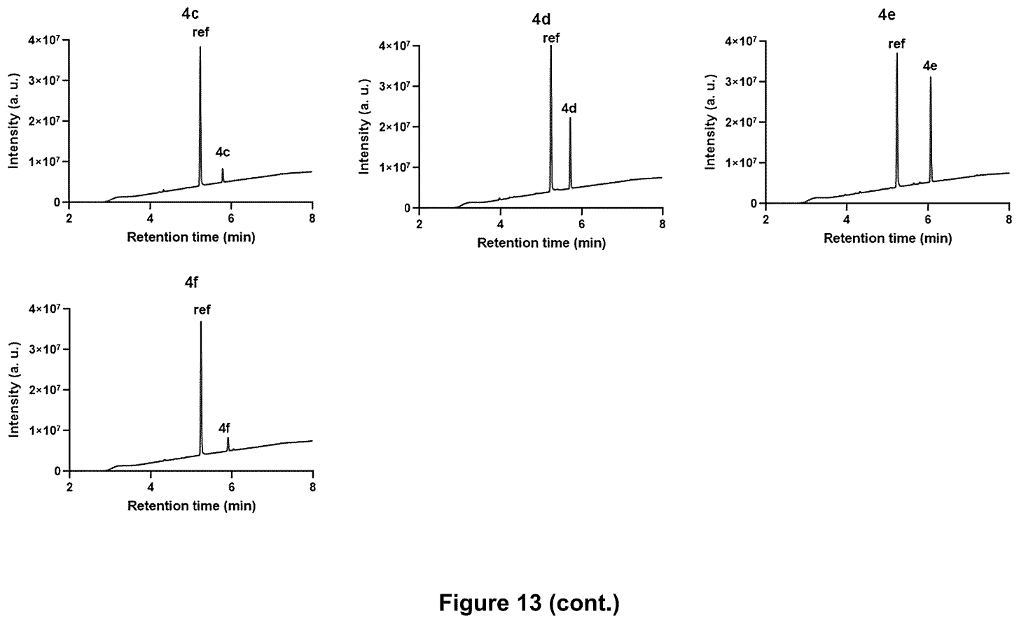

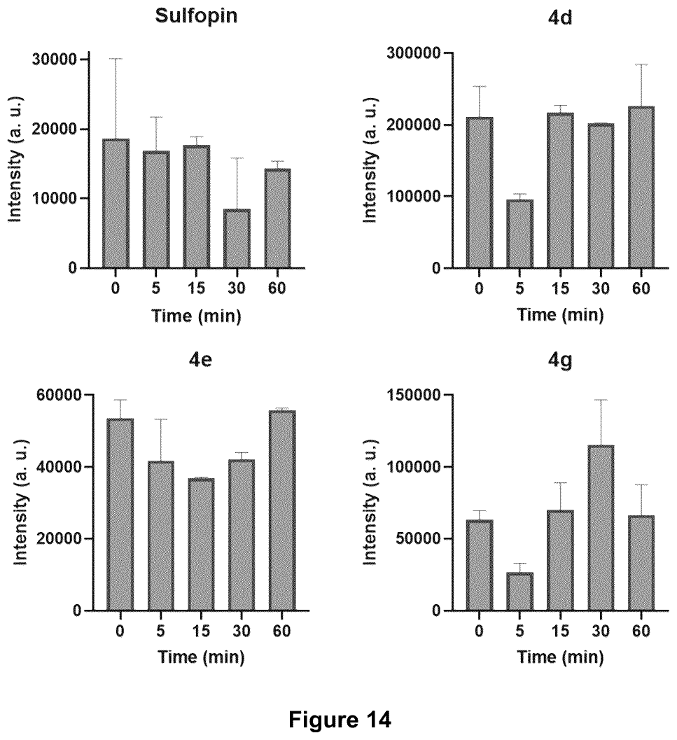

Similar to the model compounds, the chloroacetamide (Sulfopin) and sulfonate (4a) warheads show 30% and 15% hydrolysis after 2 days respectively (FIG. 13). Sulfamate compounds (4b-4g) showed high buffer stability even after four days. Moreover, both Sulfopin and the sulfamate compounds (4b-4g) showed good metabolic stability (FIG. 14).

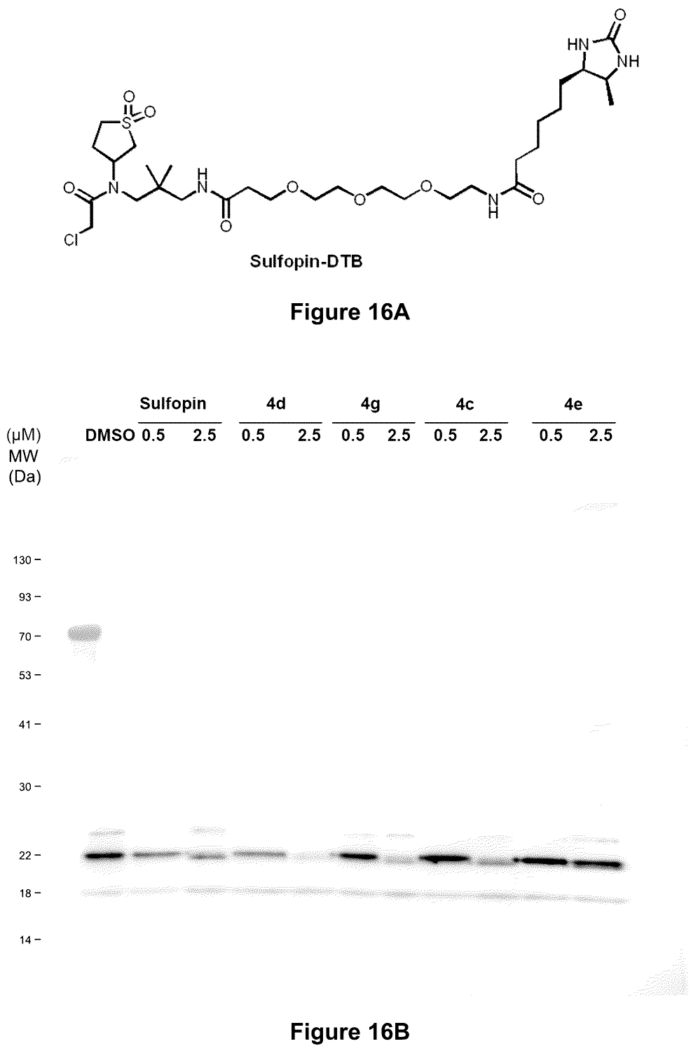

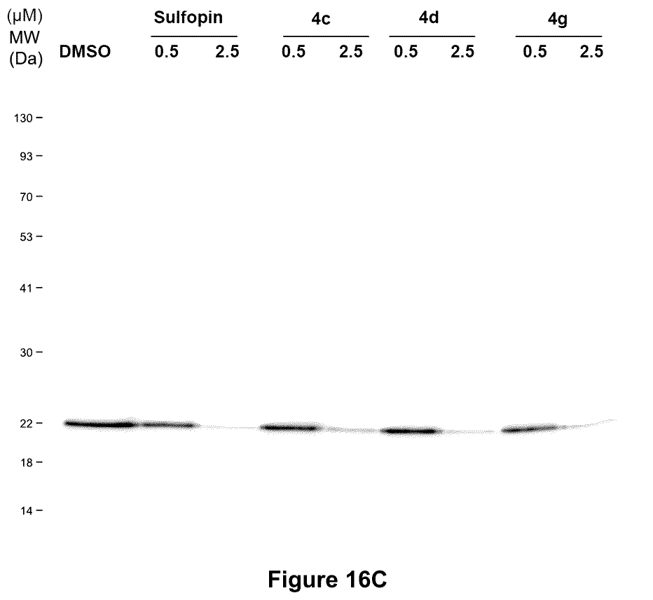

Using the previous reported results of the Applicants on Sulfopin-DTB probe [Dubiella, C. et al. Nat. Chem. Biol. 17, 954-963 (2021), which is hereby incorporated by reference in its entirety]. (FIG. 16A) the cellular engagement of these compounds was evaluated with Pin1 in OCI-AML2 cells. Compounds 4c, 4d, 4e, 4g and Sulfopin were incubated in OCI-AML2 cells (0.5 and 2.5 μM) followed by lysis and treatment with Sulfopin-DTB (1 μM). The lysates were pulled down using streptavidin beads and Pin1 was imaged by Western Blot. Similar to sulfopin, compounds 4d and 4g show ˜80% of Pin1 engagement at 2.5 μM whereas 4c and 4e show partial cellular labeling (FIG. 11D, 16B). At 0.5 μM, all these compounds only show ˜30% Pin1 engagement. To assess the selectivity of these compounds, a competitive isoTOP-ABPP experiment in PATU-8988T cells was conducted with Sulfopin as well as compound 4d. In this experiment, out of about 3,000 cysteines identified by the iodoacetamide probe, Pin1 Cys113 showed the highest competition ratio for both compounds. Both Sulfopin and compound 4d labeled only six peptides with an H/L ratio >2. Despite Pin1 being identified as the top target for both compounds, the H/L ratios for Sulfopin indicate more complete target engagement under these conditions.

While certain features and uses thereof have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure herein.

1. A Sulfamate compound represented by the structure of Formula I: 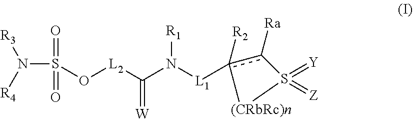
or a pharmaceutically acceptable salt thereof;
wherein,
the [symbol] represents a saturated or non-saturated bond;
Y and are each independently selected from the group consisting of O, S and NH;
each of R₂ and Ra, Rb and Rc are independently selected from the group consisting of hydrogen, alkyl, alkenyl, alkynyl, cycloalkyl, aryl, heteroaryl, heterocyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, or alternatively, R₂ is absent when the dashed line represents an unsaturated bond;
each of R₁, R₃ and R₄ are each independently selected from the group consisting of hydrogen, OH, any group that forms a urea, an amide, a thiourea, hydrazide or hydroxamic acid (via the nitrogen), substituted or unsubstituted linear or branched alkyl, alkenyl, alkynyl, substituted or unsubstituted cycloalkyl, substituted or unsubstituted heterocyclyl, substituted or unsubstituted aryl and substituted or unsubstituted heteroaryl, or R₃ and R₄ form together a five or six membered ring with the nitrogen;
n is 1, 2, 3 or 4;
L₁ is a bond, substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group;
L₂ is substituted or unsubstituted linear or branched alkylene, substituted or unsubstituted linear or branched alkenylene, substituted or unsubstituted linear or branched alkynylene, substituted or unsubstituted cycloalkyl, substituted or unsubstituted heterocyclic, substituted or unsubstituted aryl, substituted or unsubstituted heteroaryl, or an ether group;
W is O, S or NR₅; and
R₅ is selected from the group consisting of hydrogen, alkyl, alkenyl, alkynyl, cycloalkyl, heterocyclic, aryl and heteroaryl.

2. The Sulfamate compound according to claim 1, wherein the Sulfamate compound is represented by the structure of Formula II: 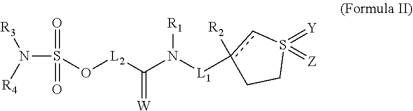
or a pharmaceutically acceptable salt thereof;
wherein,
R₁, R₂, R₃, R₄, L₁, L₂, W, Y and Z are defined in claim 1.

3. The Sulfamate compound according to claim 1, wherein the Sulfamate compound is represented by the structure of Formula III: 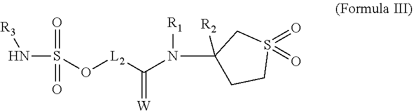
or a pharmaceutically acceptable salt thereof
wherein,
R₁, R₂, R₃, L₂ and W are defined in claim 1.

4. The Sulfamate compound according to claim 1, wherein the Sulfamate compound is represented by the structure of Formula IV: 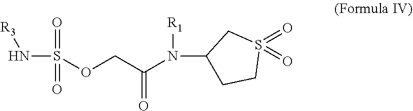
or a pharmaceutically acceptable salt thereof;
wherein,
R₁ and R₃, are as defined in claim 1.

5. The Sulfamate compound or pharmaceutically acceptable salt thereof according to claim 1, wherein n is 2.

6. The Sulfamate compound or pharmaceutically acceptable salt thereof according to claim 1, wherein Y and Z are each O (oxygen).

7. The Sulfamate compound or pharmaceutically acceptable salt thereof according to claim 1, wherein the [symbol] a saturated bond.

8. The Sulfamate compound or pharmaceutically acceptable salt thereof according to claim 1, wherein L₁ is a bond.

9. The Sulfamate compound or pharmaceutically acceptable salt thereof according to claim 1, wherein R₄ is a hydrogen.

10. The Sulfamate compound or pharmaceutically acceptable salt thereof according to claim 1, wherein L₂ is methylene.

11. The Sulfamate compound or pharmaceutically acceptable salt thereof according to claim 1, wherein R₃ is a substituted or unsubstituted alkyl, substituted or unsubstituted cycloalkyl, substituted or unsubstituted aryl, substituted or unsubstituted heteroalicyclic, or substituted or unsubstituted heteroaryl.

12. The Sulfamate compound or pharmaceutically acceptable salt thereof according to claim 11, wherein R₃ is C₁-C₁₀ alkyl, benzyl, or substituted or unsubstituted phenyl.

13. The Sulfamate compound or pharmaceutically acceptable salt thereof according to claim 11, wherein R₃ is selected from the group consisting of C₁-C₃ alkyl, benzyl, or phenyl, wherein the C₁-C₃ alkyl of R₃ is unsubstituted or substituted with halo, hydroxy, alkoxy, cyano, or oxo; and the benzyl or phenyl of R₃ is unsubstituted or substituted with halo, alkyl, hydroxy, alkoxy, or cyano.

14. The Sulfamate compound or pharmaceutically acceptable salt thereof according to claim 12, wherein said substituted phenyl is substituted with alkyl or halo.

15. The Sulfamate compound or pharmaceutically acceptable salt thereof according to claim 11, wherein R₃ is unsubstituted C₁-C₃ alkyl, unsubstituted benzyl, or phenyl, wherein the phenyl of R₃ is unsubstituted or substituted with halo or C₁-C₃ alkyl.

16. The Sulfamate compound or pharmaceutically acceptable salt thereof according to claim 11, wherein R₃ is methyl.

17. The Sulfamate compound or pharmaceutically acceptable salt thereof according to claim 11, wherein R₃ is benzyl.

18. The Sulfamate compound or pharmaceutically acceptable salt thereof according to claim 11, wherein R₃ is phenyl, unsubstituted or substituted with halo or C₁-C₃ alkyl.

19. The Sulfamate compound or pharmaceutically acceptable salt thereof according to claim 1, wherein R₁ is represented by Formula A:
—CH₂-Q′ Formula A
wherein Q′ is selected from the group consisting of alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

20. The Sulfamate compound or pharmaceutically acceptable salt thereof according to claim 19, wherein Q′ is a tertiary alkyl, alkenyl, alkynyl, cycloalkyl, or heterocyclic.

21. The Sulfamate compound or pharmaceutically acceptable salt thereof according to claim 19, wherein Q′ is a substituted or unsubstituted t-butyl or substituted or unsubstituted cycloalkyl.

22. The sulfamate compound or pharmaceutically acceptable salt thereof according to claim 19, wherein Q′ is C₄-C₆ alkyl or C₅-C₇ cycloalkyl, each of which is unsubstituted or substituted by halo, hydroxy, alkoxy, cyano, or oxo.

23. The sulfamate compound or pharmaceutically acceptable salt thereof according to claim 19, wherein the alkyl of Q′ is secondary or tertiary.

24. The sulfamate compound or pharmaceutically acceptable salt thereof according to claim 23, wherein the alkyl of Q′ is tertiary.

25. The sulfamate compound or pharmaceutically acceptable salt thereof according to claim 19, wherein Q′ is tert-butyl.

26. The Sulfamate compound or pharmaceutically acceptable salt thereof according to claim 19, wherein Q′ is cyclohexyl.

27. The Sulfamate compound or pharmaceutically acceptable salt thereof according to claim 19, wherein Q′ is a triazole.

28. The Sulfamate compound or pharmaceutically acceptable salt thereof according to claim 27, wherein said triazole has Formula B: 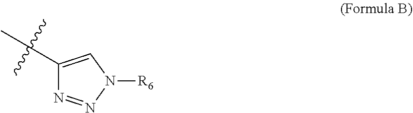
wherein R₆ is selected from the group consisting of alkyl, alkenyl, alkynyl, cycloalkyl, heteroalicyclic, aryl and heteroaryl.

29. The Sulfamate compound or pharmaceutically acceptable salt thereof according to claim 1, wherein the Sulfamate compound is represented by the structure of compound: 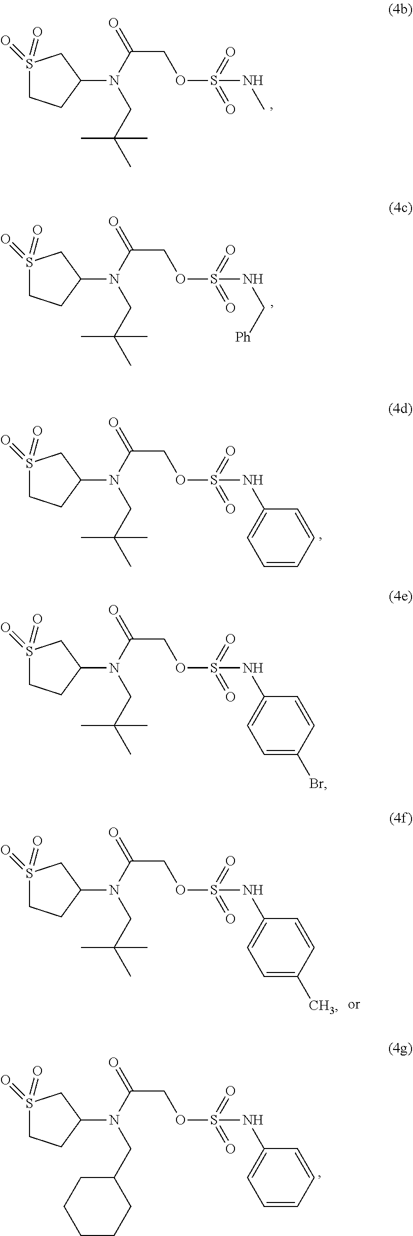
or a pharmaceutically acceptable salt thereof.

30. A method of modulating the activity of Pin1 comprising contacting Pin1 with the sulfamate compound or pharmaceutically acceptable salt thereof according to claim 1, or a pharmaceutically acceptable salt thereof.

31. The method according to claim 30, wherein a covalent bond is formed between Cys113 residue of said Pin1 and a carbon near the sulfamate group (electrophilic site), wherein releasing a sulfamic acid leaving group; and wherein hydrogen bonds are formed between Gln131 and His 157 residues of said Pin1 and the sulfur ring; and between protein Pin1 and the sulfamate group.

32. The method according to claim 30, for use in treating a condition in which modulating an activity of Pin1 is beneficial.

33. A pharmaceutical composition comprising the Sulfamate compound according to claim 1, or pharmaceutically acceptable salt thereof, and a pharmaceutically acceptable carrier or excipient.

34. A method of treating a disease or condition associated with Pin1 activity, comprising administering to a subject in need thereof the compound of claim 1, or a or pharmaceutically acceptable salt thereof, or a pharmaceutical composition of claim 33.

35. The method of claim 34, wherein the disease or condition is a proliferative disease or disorder.

36. The method of claim 34, wherein the disease is a cancer.

37. The method of claim 34, wherein the disease is selected from the group consisting of a pancreatic cancer, a neuroblastoma, a prostate cancer, an ovarian carcinoma, and a breast adenocarcinoma.

38. The method of claim 34, wherein the disease or condition is an immune disease or disorder.
